(19) United States
(12) Patent Application Publication
(10) Pub. No.: US 2025/0188062 A1
(43) Pub. Date: Jun. 12, 2025

(54) TRIAZINE COMPOUNDS AND METHODS OF MAKING AND USING THE SAME

(71) Applicant: Athos Therapeutics, Inc., Torrance, CA (US)

(72) Inventors: Dimitrios Iliopoulos, Los Angeles, CA (US); David G. Ho, Monterey Park, CA (US); Iordanis Karagiannidis, North Hollywood, CA (US); Phithi Nguyen, Anaheim, CA (US); Dimitra Chalkia, Fullerton, CA (US)

(21) Appl. No.: 18/262,712

(22) PCT Filed: Jan. 24, 2022
(86) PCT No.: PCT/US2022/070321

§ 371 Date: Jul. 24, 2023

Related U.S. Application Data
(60) Provisional application No. 63/141,316, filed on Jan. 25, 2021.

(51) Int. Cl. C07D 403/14; A61K 31/53; A61K 31/5377; A61K 31/55; A61K 31/551; A61P 1/00; C07D 401/14; C07D 403/04; C07D 405/14; C07D 409/14; C07D 413/14; C07D 491/107

(52) U.S. Cl. CPC C07D 403/14; A61K 31/53; A61K 31/5377; A61K 31/55; A61K 31/551; A61P 1/00; C07D 401/14; C07D 403/04; C07D 405/14; C07D 409/14; C07D 413/14; C07D 491/107

(57) ABSTRACT

Disclosed herein are triazine compounds (e.g., triazines and pyrazole triazines) and methods of treating diseases and/or conditions (e.g., inflammation) with the triazine compounds disclosed herein. In several embodiments, the disclosed triazine compounds are used for the inhibition of Jumonji domain-containing protein D3 (JMJD3) and/or the treatment of JMJD3 related disease states.

### CROSS REFERENCE TO RELATED APPLICATIONS

The present application claims the benefit of priority to U.S. Provisional Patent Application No. 63/141,316, filed Jan. 25, 2021. The disclosure of the foregoing application is hereby incorporated by reference in its entirety. Any and all applications for which a foreign or domestic priority claim is identified in the Application Data Sheet as filed with the present application are hereby incorporated by reference under 37 CFR 1.57.

### FIELD

Disclosed herein are triazine compounds (e.g., triazines) including functionalized triazine compounds (e.g., pyrazole triazines), pharmaceutical compositions that include the same, methods of using the same, and methods of making the disclosed triazine compounds.

### BACKGROUND

The Jumonji protein is the founding member of a group of proteins characterized by a novel structural motif, the JmjC domain. This is an extensive group of demethylase enzymes which can be defined into several families according to sequence similarity within the JmjC domain and the presence of other domains in the full length protein. The JmjC domain of several members of this family has been shown to possess lysine demethylation activity, which is dependent on iron (Fe (II)) and aketoglutarate as co-factors. Unlike LSD1, which can only remove mono- and dimethyl lysine modifications, the JmjC-domain-containing histone demethylases (JHDMs) can remove all three histone lysine-methylation states. Jumonji domain-containing protein D3 (JMJD3) is a member of this family of JmjC histone demethylases.

### SUMMARY

Several embodiments disclosed herein pertain to triazine compounds and their use as inhibitors of the Jumonji protein, including JMJD3. Some embodiments pertain to methods of manufacture such compounds. Some embodiments pertain to methods of use of these compounds as therapeutics for treating disease states (e.g., inflammatory diseases, autoimmune diseases, and cancer). In several embodiments, the triazine compound comprises a substituted triazine bonded to a heteroaryl group. In several embodiments, the heteroaryl is a pyrazole (e.g., pyrazolyl). In several embodiments, the heteroaryl group is a pyridinyl. In several embodiments, by using one or more triazine compounds to inhibit the activity of JMJD3 in a subject, a disease state can be treated.

Several embodiments include a triazine compound, its pharmaceutically acceptable salts, enantiomers, methods of manufacture, and/or its method of use in treating disease states. In several embodiments, the triazine compound (or pharmaceutical composition comprising the triazine compound) comprises, consists of, or consists essentially of the structure of Formula (I) (or any other structure disclosed herein), its pharmaceutically acceptable salts, and/or enantiomers. Several embodiments pertain to methods of manufacture of a compound of Formula (I) and/or methods of use of a compound of Formula (I) in treating disease states. In several embodiments, by using one or more compounds of Formula (I) (or any other structure disclosed herein) to inhibit the activity of JMJD3 in a subject, a disease state can be treated. In several embodiments, the disease state is associated with inflammation. In several embodiments, the disease state is associated with an autoimmune disorder.

Several embodiments pertain to a compound, or a pharmaceutically acceptable salt thereof, having a structure represented by Formula I:

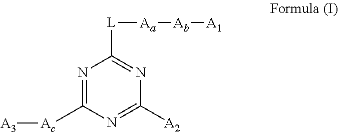

where L is selected from the group consisting of —C(R¹)₂—, —N—, —NR¹—, —N(R¹)₂, optionally substituted amine(alkyl), —O—, —C(═O)—, —OC(═O)—, —C(═O)O—, and optionally substituted heterocyclyl, or L is not present; Aₐ is selected from the group consisting of optionally substituted C₁-C₆ alkylene and C₁-C₆ alkyl, or Aₐ is not present; A_{b} is selected from the group consisting of —C(═O)— and —N(CH₃)—, or A_{b} is not present; A₁ is selected from the group consisting of —H, hydroxyl, amino, optionally substituted C₁₋₁₂ alkyl, optionally substituted C₁-C₁₂alkoxy, optionally substituted C₁-C₁₂ alkamino, optionally substituted carbamide, C-amido, optionally substituted C₆₋₁₀ aryl, optionally substituted 5-12 membered heteroaryl, optionally substituted C₃₋₁₀ carbocyclyl, and 3-12 membered heterocyclyl, or A₁ is not present; A₂ is selected from the group consisting of optionally substituted C₆₋₁₀ aryl, optionally substituted 5-12 membered heteroaryl, and optionally substituted 3-12 membered heterocyclyl; A_{c} is selected from the group consisting of C₁₋₃ alkylene, C₁-C₃ alkamino, and —O—, or A_{c} is not present; A₃ is selected from the group consisting of —H, optionally substituted C₁₋₁₂ alkyl, optionally substituted C₁-C₁₂ alkoxy, optionally substituted C₁-C₁₂ alkamino, optionally substituted C₆₋₁₀ aryl, optionally substituted 5-12 membered heteroaryl, optionally substituted C₃₋₁₀ carbocyclyl, and optionally substituted 3-12 membered heterocyclyl; and each instance of R¹, where present, is independently selected from the group consisting of —H, halogen, hydroxy, C₁₋₆ alkyl, C₁-C₆ alkoxy, C₁₋₆ haloalkyl, and C₆₋₁₀ aryl, or, where two R¹ groups are present, the R¹ groups taken together form an optionally substituted 3-12 membered heterocycle or an optionally substituted C₃₋₁₀ carbocyclyl.

In several embodiments, L is —N(R¹)₂ and the R¹ groups taken together form a 3-12 membered heterocyclyl group. In several embodiments, L is a 6-membered heterocyclyl group comprising 1 or 2 heteroatoms. In several embodiments, the heteroatoms of L are N. In several embodiments, Aₐ and A_{b} are not present. In several embodiments, A₁ is a 3-12 membered heterocyclyl group. In several embodiments, a ring of A₁ and a ring of L provide a spiro ring system. In several embodiments, L, Aₐ, A_{b}, and A₁ together provide a structure selected from the group consisting of:

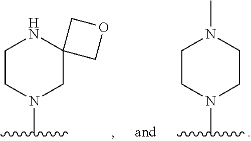

In several embodiments, L is —NH—. In several embodiments, Aₐ is C₁-C₆ alkylene. In several embodiments, Aₐ is C₁-C₃ alkylene. In several embodiments, A_{b} is —C(═O)— or is not present. In several embodiments, A₁ is amino, hydroxyl, C₁₋₃ alkyl, carbamide optionally substituted with methyl or ethyl, 3 to 6 membered heterocyclyl, 3 to 6 membered heteroaryl, or C₁-C₃ alkoxy. In several embodiments, L, Aₐ, A_{b}, and A₁ together provide a structure selected from the group consisting of:

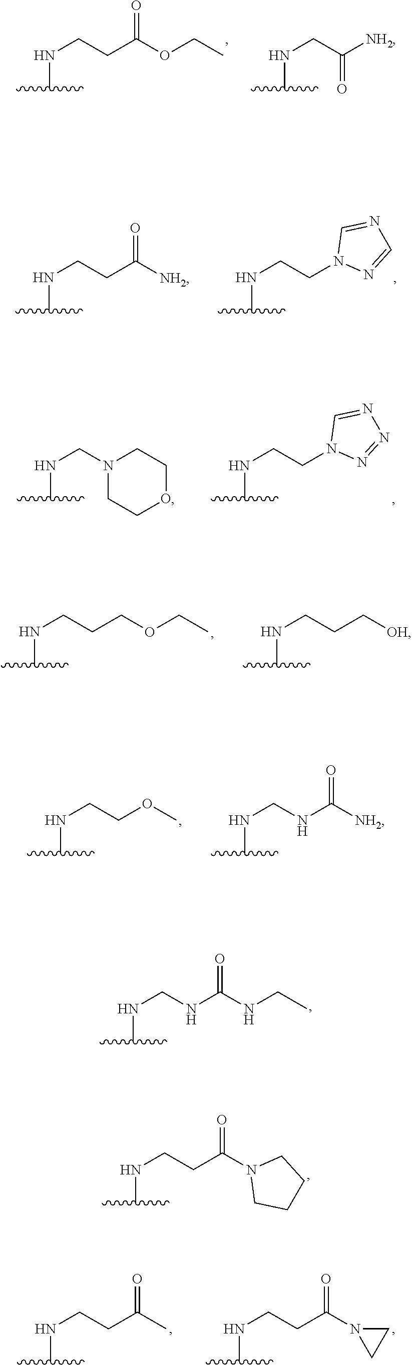

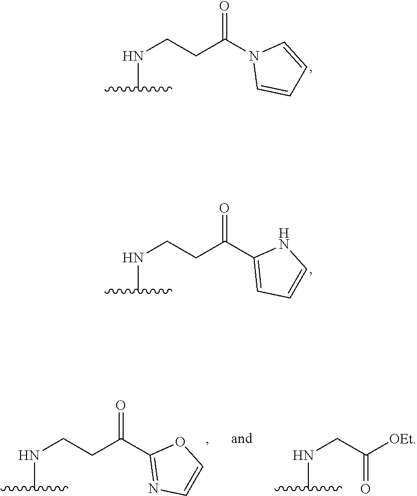 [SMILES: *NCCC(=O)n1cccc1 ; *NCCC(=O)c1ccc[nH]1 ; *NCCC(=O)c1ncco1 ; *NCC(=O)OCC]

Any one of the foregoing structures may be further optionally substituted by replacing one or more —H atoms of any carbon or nitrogen atom present within the L, Aₐ, A_{b}, and A₁ structure.

In several embodiments, Aₐ is not present. In several embodiments, A_{b} is not present. In several embodiments, A₁ is C₁₋₄ alkyl optionally substituted with hydroxyl or halogen, optionally substituted C₃₋₆ carbocyclyl, 3 to 6 membered heterocyclyl, 3 to 6 membered aryl optionally substituted with halogen, or optionally substituted 3 to 6 membered heteroaryl. In several embodiments, L, Aₐ, A_{b}, and A₁ together provide a structure selected from the group consisting of:

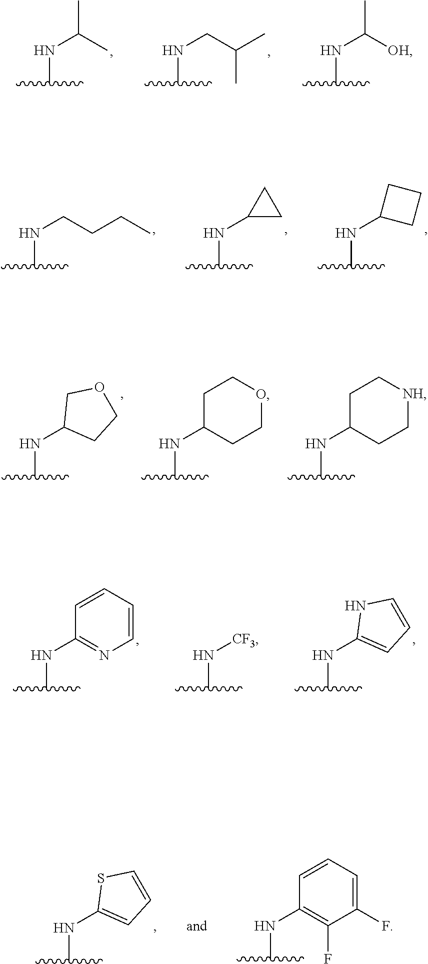

Any one of the foregoing structures may be further optionally substituted by replacing one or more —H atoms of any carbon or nitrogen atom present within the L, Aₐ, A_{b}, and A₁ structure.

In several embodiments, A₂ is selected from the group consisting of optionally substituted 5-6 membered heteroaryl or optionally substituted 5-6 membered heterocyclyl. In several embodiments, A₂ is a structure selected from the group consisting of:

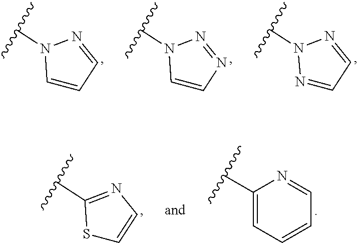 [SMILES: *n1cccn1 ; *n1ccnn1 ; *n1nccn1 ; *c1nccs1 ; *c1ccccn1]

Any one of the foregoing structures may be further optionally substituted by replacing one or more —H atoms of any carbon or nitrogen atom present within the A₂ structure.

In several embodiments, A_{c} is —O— or is not present. In several embodiments, A₃ is selected from the group consisting of C₁₋₅ alkyl, phenyl optionally substituted with —F, C₁-C₃ alkamino, optionally substituted 5-12 membered heteroaryl, optionally substituted C₃₋₁₀ carbocyclyl, and optionally substituted 3-12 membered heterocyclyl. In several embodiments, A_{c} and A₃ together provide a structure selected from the group consisting of

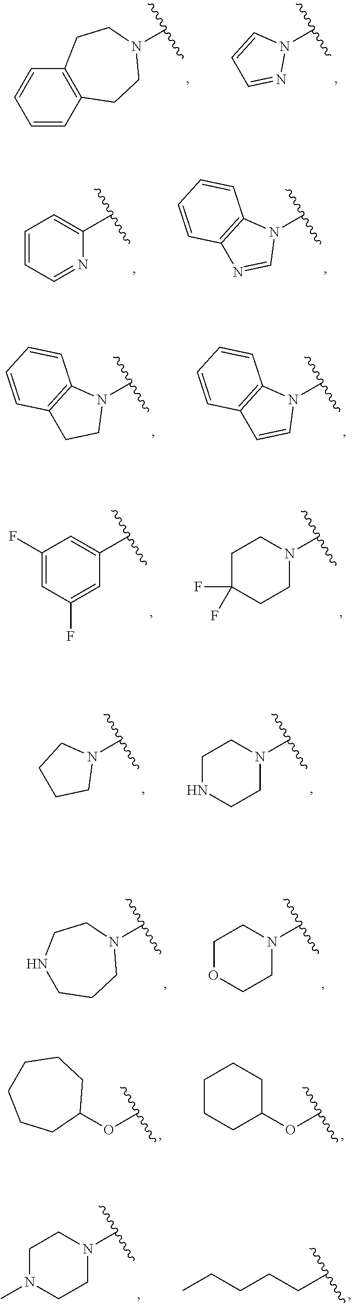

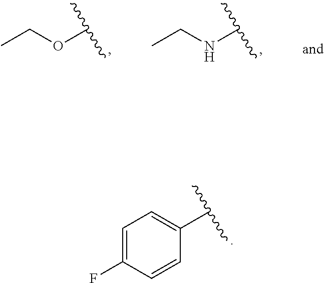

Any one of the foregoing structures may be further optionally substituted by replacing one or more —H atoms of any carbon or nitrogen atom present within the A_{c} and A₃ structure.

In several embodiments, A_{c} is C₁₋₃ alkylene or C₁-C₃ alkamino. In several embodiments, A₃ is selected from the group consisting of phenyl optionally substituted with —F and optionally substituted C₃₋₇ carbocyclyl. In several embodiments, A_{c} and A₃ together provide a structure selected from the group consisting of

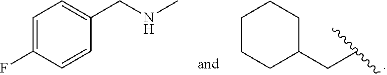

Any one of the foregoing structures may be further optionally substituted by replacing one or more —H atoms of any carbon or nitrogen atom present within the A_{c} and A₃ structure.

Some embodiments pertain to a compound of Formula (I) or a pharmaceutically acceptable salt thereof having a structure represented by Formula I:

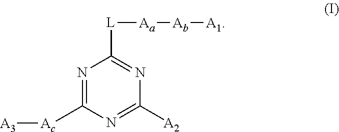

In several embodiments, L is selected from the group consisting of —C(R¹)₂—, —N—, —NR¹—, —N(R¹)₂, optionally substituted amine(alkyl), —O—, —C(═O)—, —OC(═O)—, —C(═O)O—, and a single bond. In several embodiments, Aₐ is selected from the group consisting of optionally substituted C₁-C₆ alkylene and C₁-C₆ alkyl. In several embodiments, Aₐ is not present. In several embodiments, A_{b} is selected from the group consisting of —C(═O)— or —N(CH₃)—. In several embodiments, A_{b} is not present. In several embodiments, A₁ is selected from the group consisting of hydroxyl, amino, optionally substituted C₁₋₁₂ alkyl, optionally substituted C₁-C₁₂ alkoxy, optionally substituted C₁-C₁₂ alkamino, optionally substituted carbamide, C-amido, optionally substituted C₆₋₁₀ aryl, 5-12 membered heteroaryl, C₃₋₁₀ carbocyclyl, and 3-12 membered heterocyclyl. In several embodiments, A₁ is not present. In several embodiments, A₂ is selected from the group consisting of C₆₋₁₀ aryl, 5-12 membered heteroaryl, and 3-12 membered heterocyclyl. In several embodiments, A_{c} is selected from the group consisting of C₁₋₃ alkylene, C₁-C₃ alkamino, —O—, and a single bond. In several embodiments, A₃ is selected from the group consisting of optionally substituted C₁₋₁₂ alkyl, C₁-C₁₂ alkoxy, optionally substituted C₁-C₁₂ alkamino, optionally substituted C₆₋₁₀ aryl, 5-12 membered heteroaryl, and optionally substituted 3-12 membered heterocyclyl. In several embodiments, each instance of R¹, where present, is independently selected from the group consisting of —H, halogen, hydroxy, C₁₋₆ alkyl, C₁-C₆ alkoxy, C₁₋₆ haloalkyl, and C₆₋₁₀ aryl. In several embodiments, where two R¹ groups are present, the R¹ groups taken together form a 3-12 membered heterocycle or a C₃₋₁₀ carbocyclyl.

In several embodiments, when any one of Aₐ, A_{b}, A₁, A₂, A₃ is substituted with one or more optional substitutions, each of the one or more optional substitutions are independently selected from the group consisting of —OH, C₁₋₃ alkyl, C₁₋₃ alkoxy, or halogen. In several embodiments, when the A₁ group is substituted with one or more optional substitutions, the one or more optional substitutions are independently selected from the group consisting of acyl, C₁₋₆ alkyl, C₁₋₆ alkoxy, C-amido, aryl, ester, halogen, heteroaryl, heterocyclyl, and hydroxy. In several embodiments, when the A₃ group is substituted with one or more optional substitutions, the one or more optional substitutions are independently selected from the group consisting of C₁₋₆ alkyl, C₁₋₆ cycloalkyl, C₆₋₁₀ aryl, halogen, and hydroxy.

In several embodiments, the compound of Formula I is further represented by the structure of Formula Ia:

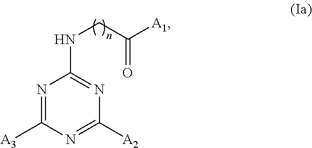

and n is an integer equal to 1, 2, or 3.

In several embodiments, the compound of Formula (I) is further represented by the structure of Formula Ib:

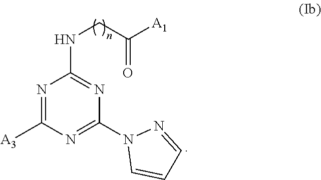

In several embodiments, definitions for like variables in different formulae (A₂ for Formula (I) and Formula (Ia); A₃ for Formula (I) and Formula (Ib), etc.) maybe used to define that like variable for any formula where the variable occurs. Thus, any definition of a variable for Formula (I) may be defined using that same variable for any one or more of Formula (Ia), (Ib), (Ic), (Id), and (Ie) (or vice versa).

In several embodiments, the compound of Formula (I) is further represented by one of the following:

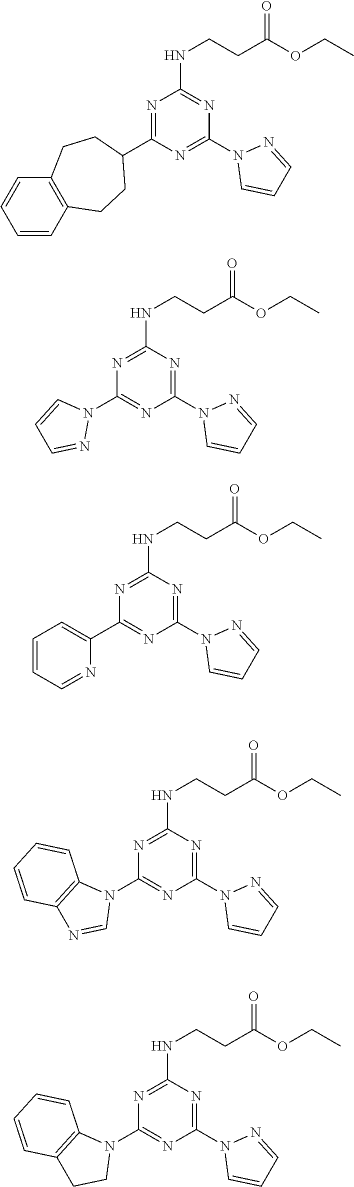

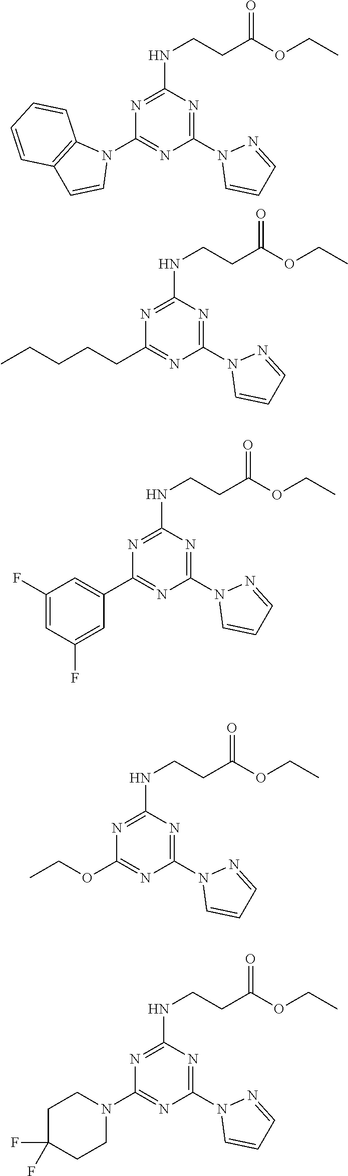

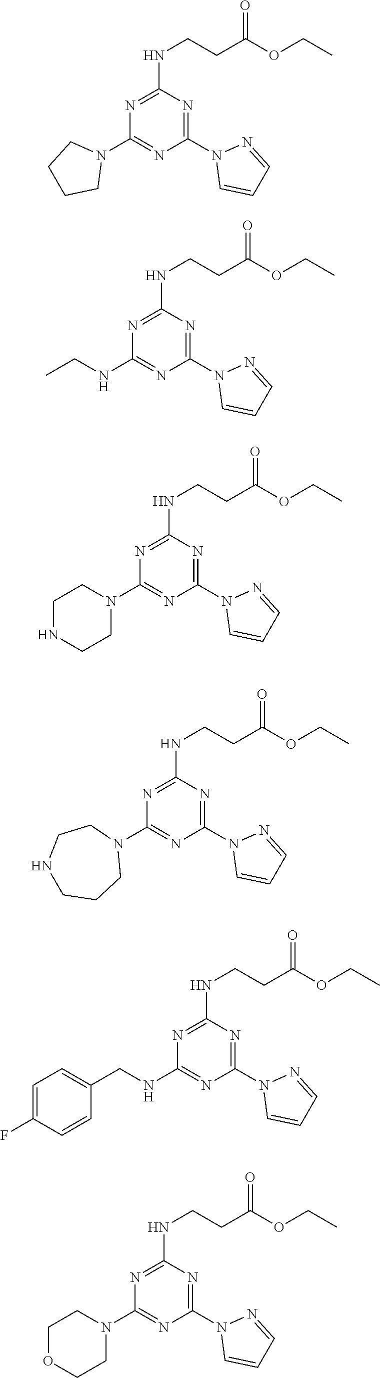

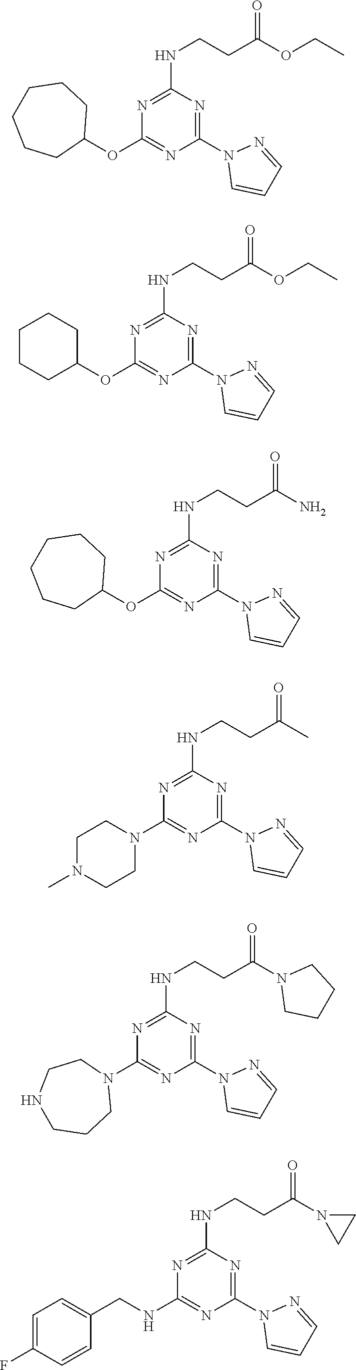

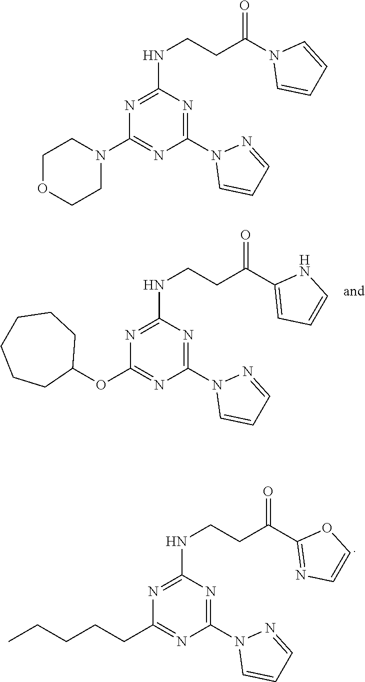

In several embodiments, the compound of Formula I is further represented by the structure of Formula Ic:

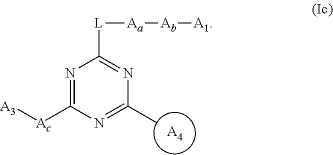

In several embodiments, a ring comprising A₄ is a 5 or 6 membered heteroaryl. In several embodiments, the compound of Formula I is further represented by one of the following:

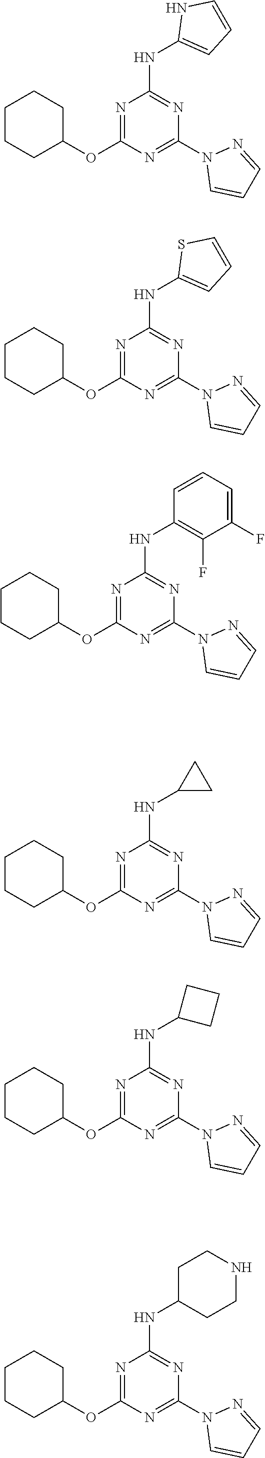

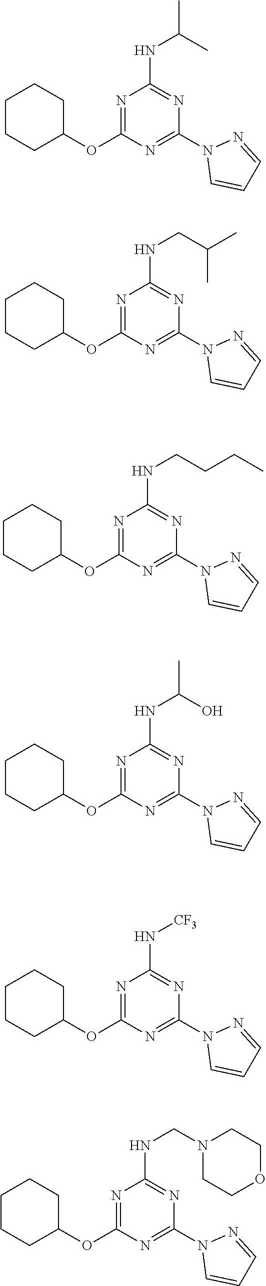

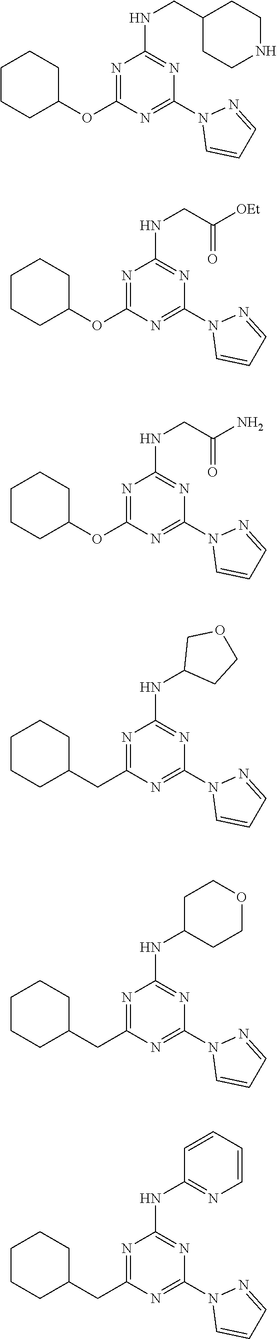 [SMILES: c1cnn(-c2nc(NCC3CCNCC3)nc(OC3CCCCC3)n2)c1 ; CCOC(=O)CNc1nc(OC2CCCCC2)nc(-n2cccn2)n1 ; NC(=O)CNc1nc(OC2CCCCC2)nc(-n2cccn2)n1 ; c1cnn(-c2nc(CC3CCCCC3)nc(NC3CCOC3)n2)c1 ; c1cnn(-c2nc(CC3CCCCC3)nc(NC3CCOCC3)n2)c1 ; c1ccc(Nc2nc(CC3CCCCC3)nc(-n3cccn3)n2)nc1]

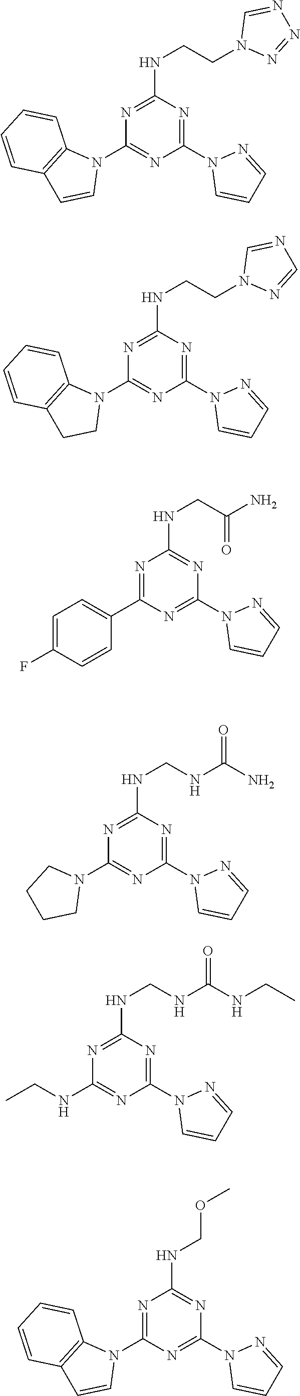

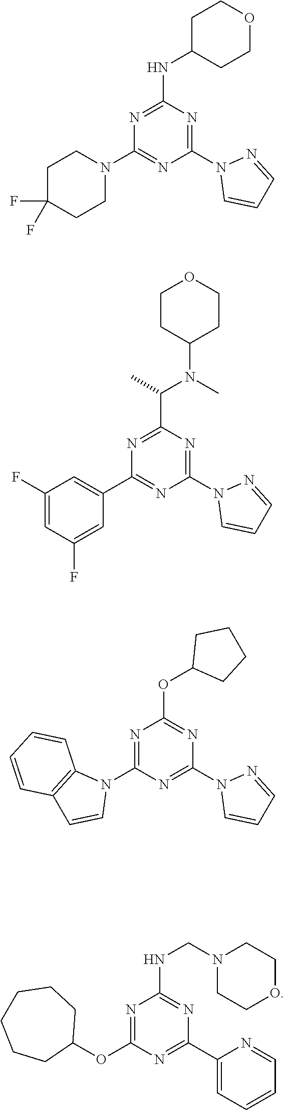 [SMILES: FC1(F)CCN(c2nc(NC3CCOCC3)nc(-n3cccn3)n2)CC1 ; C[C@@H](c1nc(-c2cc(F)cc(F)c2)nc(-n2cccn2)n1)N(C)C1CCOCC1 ; c1ccc2c(c1)ccn2-c1nc(OC2CCCC2)nc(-n2cccn2)n1 ; c1ccc(-c2nc(NCN3CCOCC3)nc(OC3CCCCCC3)n2)nc1]

In several embodiments, the compound of Formula I is further represented by the structure of Formula Id:

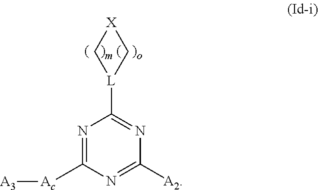

In several embodiments, X is selected from the group consisting of —CH₂—, —NH—, and —O—; m is an integer equal to 1, 2, 3, or 4; and o is an integer equal to 1, 2, 3, or 4. In several embodiments, the compound of Formula I, represented by:

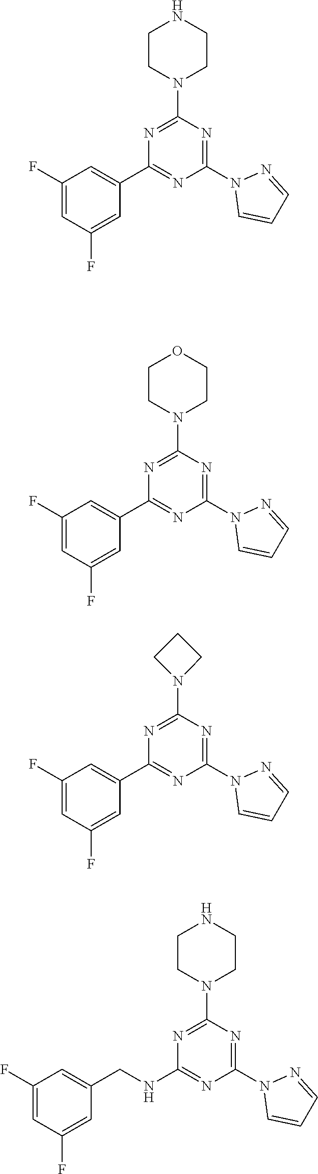 [SMILES: Fc1cc(F)cc(-c2nc(N3CCNCC3)nc(-n3cccn3)n2)c1 ; Fc1cc(F)cc(-c2nc(N3CCOCC3)nc(-n3cccn3)n2)c1 ; Fc1cc(F)cc(-c2nc(N3CCC3)nc(-n3cccn3)n2)c1 ; Fc1cc(F)cc(CNc2nc(N3CCNCC3)nc(-n3cccn3)n2)c1]

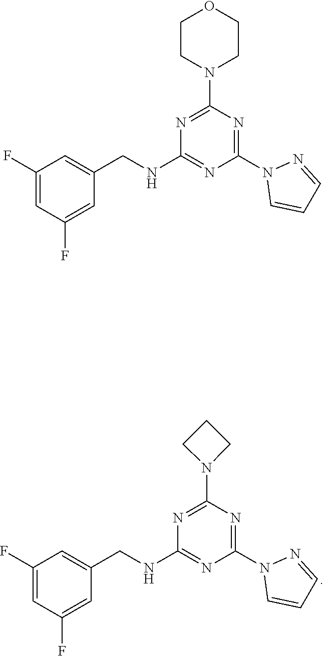 [SMILES: Fc1cc(F)cc(CNc2nc(N3CCOCC3)nc(-n3cccn3)n2)c1 ; Fc1cc(F)cc(CNc2nc(N3CCC3)nc(-n3cccn3)n2)c1]

In several embodiments, the compound of Formula I is further represented by the structure of Formula Ie:

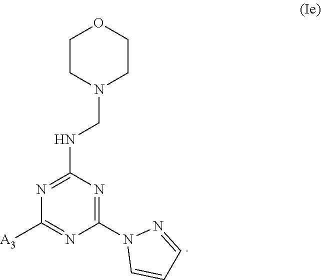

In several embodiments, the compound of Formula I, represented by:

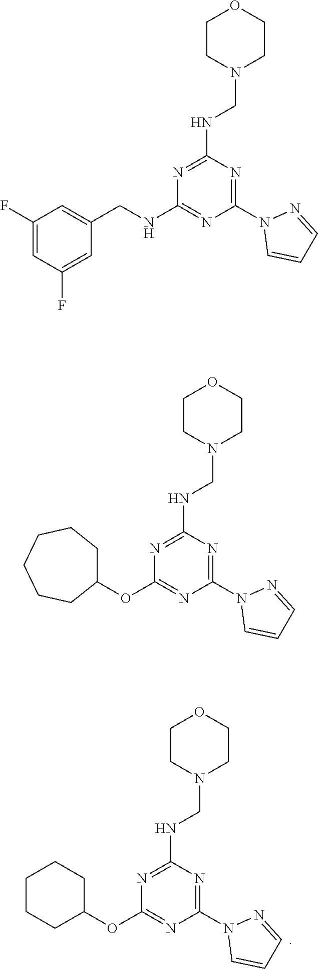 [SMILES: Fc1cc(F)cc(CNc2nc(NCN3CCOCC3)nc(-n3cccn3)n2)c1 ; c1cnn(-c2nc(NCN3CCOCC3)nc(OC3CCCCCC3)n2)c1 ; c1cnn(-c2nc(NCN3CCOCC3)nc(OC3CCCCC3)n2)c1]

In several embodiments, A₁ is selected from the group consisting of CH₃—, CF₃—, CH₃(CH₂)₃—, (CH₃)₂CH—, (CH₃)₂CHCH₂—, CH₃(CH₂)₃—, CH₃(HO)CH—, HOCH₂—, CH₃CH₂OCH₂—, NH₂C(═O)CH₂—, CH₃O—, CH₃CH₂O—, NH₂—, NH₂C(═O)NH—, and CH₃CH₂NHC(═O)NH—. In several embodiments, A₁ is selected from the group consisting of:

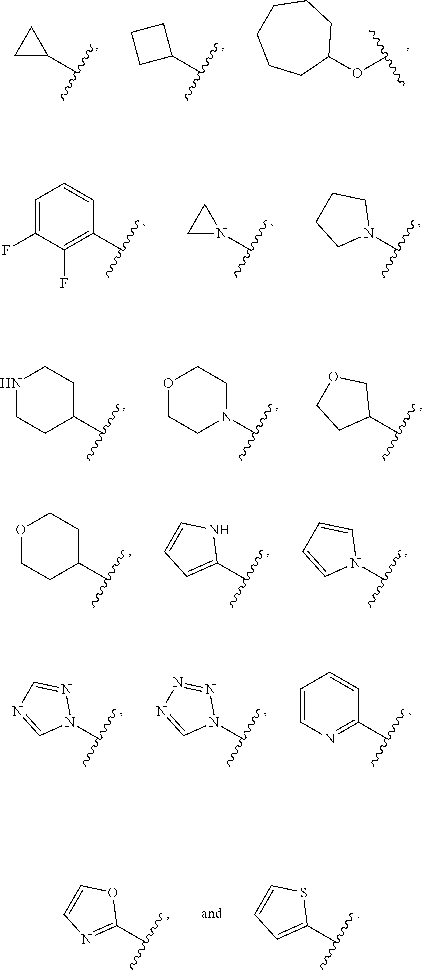

In several embodiments, A₂ is selected from the group consisting of pyrazole and pyridine.

In several embodiments, A₃ is selected from the group consisting of:

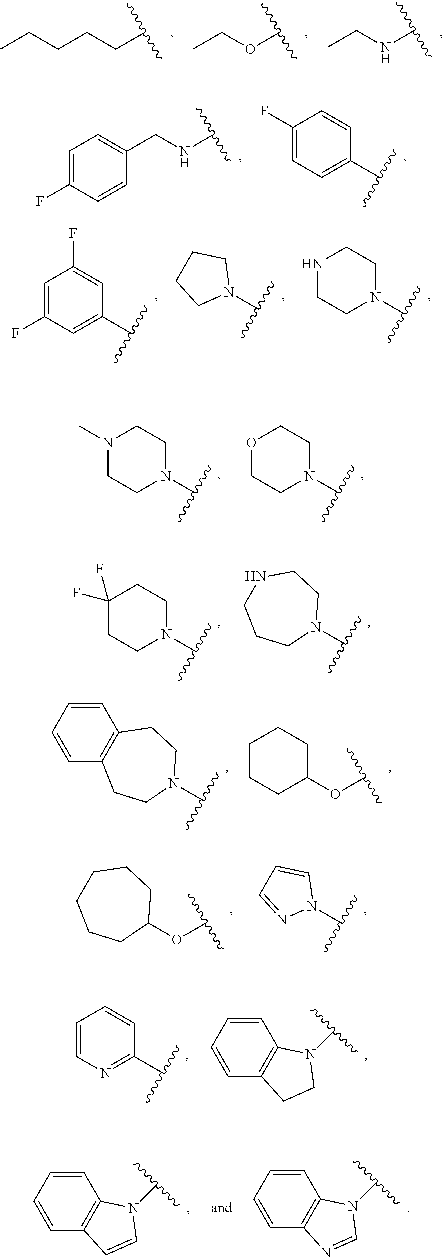

Several embodiments pertain to a method of inhibiting the activity of Jumonji proteins, including JMJD3. In several embodiments, the triazine compounds disclosed herein suppress (and are used for the suppression of) one or more of TNF-alpha (TNFA), interleukin 6 (IL6), interleukin 1beta (IL1B), interleukin 8 (IL8), interleukin 5 (IL5) and interleukin 13 (IL13). In several embodiments, the triazine compounds disclosed herein are suitable for use in methods of treating autoimmune diseases, including Ulcerative colitis, Crohn's Disease, Rheumatoid Arthritis, Systemic Lupus Erythematosus, Atopic Dermatitis, Psoriasis, Type 1 Diabetes, Graft-versus-host-disease (GVHD), Autoimmune Encephalitis, Bechet's Disease, Sjogren's Syndrome, asthma, lung allergies, and atopic dermatitis. In several embodiments, the triazine compounds disclosed herein are suitable for use in methods of treating Lupus, reumatoid arthritis, atopic dermatitis, and psoriasis. In several embodiments, the method comprises administering a triazine compound as disclosed herein (e.g., a pyrazolyl triazine) to a patient in need of treatment thereby treating the patient. In several embodiments, in response to a determination of the presence of inflammatory and autoimmune disease in a sample from a subject, the subject is administered an effective amount the compound, thereby treating the inflammatory and autoimmune disease in the subject.

In several embodiments, the triazine compounds disclosed herein are suitable for use in methods of treating cancer (e.g., kidney, breast, prostate, skin, hemopoietic system, myelodysplastic syndrome (MDS), esophageal squamous cell carcinoma, head and neck squamous cell carcinoma (HNSCC), and ovarian cancer). In several embodiments, the method comprises administering a triazine compound as disclosed herein (e.g., a pyrazolyl triazine) to a patient in need of treatment thereby treating the patient. In several embodiments, in response to a determination of the presence of cancer in a sample from a subject, the subject is administered an effective amount the compound, thereby treating the cancer in the subject.

### BRIEF DESCRIPTION OF THE DRAWINGS

FIG. 1A provides reaction pathways for the preparation of several embodiments of compounds of Formula (I).

FIG. 1B is a scheme that provides three synthetic routes for the preparation of several embodiments of compounds of Formula (I).

FIG. 2 is a scheme that provides additional synthetic routes for the preparation of several embodiments of compounds of Formula (I).

FIG. 3 provides data for a cellular assay for FoxP3 induction activity of JMJD3 inhibitors.

FIGS. 4A-4C provide cellular data for compounds disclosed herein. Compounds as disclosed herein provided suppression of TNF-alpha (TNFA) expression after LPS (lipopolysaccharide) treatment, in THP-1 human monocytes. Data for JMJD3 inhibitors Compound 89, Compound 91 are provided in FIG. 4A (p<005), Compound 97, Compound 92, and Compound 93 in FIG. 4B (p<0.01), and Compound 94 in FIG. 4C (p<0.01).

### DETAILED DESCRIPTION

Several embodiments disclosed herein provide compounds useful in inhibiting the activity of JMJD3 in a subject. Several embodiments also provide methods of treating diseases utilizing these compounds or pharmaceutical compositions comprising these compounds. In several embodiments, the compounds are triazine compounds. In several embodiments, multiple functionalities are bound to a core triazine structure including a heteroaryl (e.g., a pyrazolyl, pyridinyl, etc., bonded to the triazine ring). In several embodiments, the disclosed triazines can be used in methods of treating inflammatory and autoimmune diseases. In several embodiments, the disclosed triazines can be used in methods of treating cancer. The following description provides context and examples, but should not be interpreted to limit the scope of the inventions covered by the claims that follow in this specification or in any other application that claims priority to this specification. No single component or collection of components is essential or indispensable. For example, in some embodiments where a structure is disclosed, one or more variables of that structure may be omitted (such as R¹, L, Aₐ, A_{b}, etc.). Any feature, structure, component, material, step, or method that is described and/or illustrated in any embodiment in this specification can be used with or instead of any feature, structure, component, material, step, or method that is described and/or illustrated in any other embodiment in this specification.

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. Conventional procedures for the selection and preparation of suitable prodrug derivatives are described, for example, in Design of Prodrugs, (ed. H. Bundgaard, Elsevier, 1985), which is hereby incorporated herein by reference in its entirety.

The term “pro-drug ester” refers to derivatives of the compounds disclosed herein formed by the addition of any of several ester-forming groups that are hydrolyzed under physiological conditions. Examples of pro-drug ester groups include pivoyloxymethyl, acetoxymethyl, phthalidyl, indanyl and methoxymethyl, as well as other such groups known in the art, including a (5-R-2-oxo-1,3-dioxolen-4-yl)methyl group. Other examples of pro-drug ester groups can be found in, for example, T. Higuchi and V. Stella, in “Pro-drugs as Novel Delivery Systems”, Vol. 14, A.C.S. Symposium Series, American Chemical Society (1975); and “Bioreversible Carriers in Drug Design: Theory and Application”, edited by E. B. Roche, Pergamon Press: New York, 14-21 (1987) (providing examples of esters useful as prodrugs for compounds containing carboxyl groups). Each of the above-mentioned references is herein incorporated by reference in their entirety.

“Metabolites” of the compounds disclosed herein include active species that are produced upon introduction of the compounds into the biological milieu.

“Solvate” refers to the compound formed by the interaction of a solvent and a compound described herein, a metabolite, or salt thereof. Suitable solvates are pharmaceutically acceptable solvates including hydrates.

The term “pharmaceutically acceptable salt” refers to salts that retain the biological effectiveness and properties of a compound, which are not biologically or otherwise undesirable for use in a pharmaceutical. In many cases, the compounds herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

When referring to numerical values, the terms “or ranges including and/or spanning the aforementioned values” (and variations thereof) is meant to include any range that includes or spans the aforementioned values. For example, when the temperature of a reaction is expressed as “20° C., 30° C., 40° C., 50° C., or ranges including and/or spanning the aforementioned values,” this includes the particular temperature provided or temperature ranges spanning from 20° C. to 50° C., 20° C. to 40° C., 20° C. to 30° C., 30° C. to 50° C., 30° C. to 40° C., or 40° C. or 50° C.

As used herein, “Cₐ to C_{b}” in which “a” and “b” are integers refer to the number of carbon atoms in an alkyl, alkenyl or alkynyl group, or the number of carbon atoms in the ring of a cycloalkyl, cycloalkenyl, cycloalkynyl, aryl, or heteroaryl group. That is, the alkyl, alkenyl, alkynyl, ring of the cycloalkyl, ring of the cycloalkenyl, ring of the cycloalkynyl, ring of the aryl, or the ring of the heteroaryl can contain from “a” to “b”, inclusive, carbon atoms. Thus, for example, a “C₁ to C₄ alkyl” group refers to all alkyl groups having from 1 to 4 carbons (e.g., 1, 2, 3, or 4), that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. A “C₁ to C₆ alkyl” group refers to all alkyl groups having from 1 to 6 carbons (e.g., 1, 2, 3, 4, 5, or 6). If no “a” and “b” are designated with regard to an alkyl, alkenyl, alkynyl, cycloalkyl cycloalkenyl, cycloalkynyl, aryl, or heteroaryl group, the ranges described in these definitions are included (including the broadest ranges).

As used herein, “alkyl” refers to a straight or branched hydrocarbon chain that is fully saturated (i.e., contains no double or triple bonds). Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 20 carbon atoms (as disclosed elsewhere herein, whenever it appears herein, a numerical range such as “1 to 20” refers to each integer in the given range; for example, “1 to 20 carbon atoms” means that the alkyl group may consist of 1, 2, 3, 4, 5, 6, 7, 8, 9, 10, 11, 12, 13, 14, 15, 16, 17, 18, 19, or 20 carbon atoms, although the present definition also covers the occurrence of the term “alkyl” where no numerical range is designated). The “alkyl” group may also be a medium size alkyl having 1 to 12 carbon atoms. The “alkyl” group could also be a lower alkyl having 1 to 6 carbon atoms. The alkyl group of the compounds may be designated as “C₁₋₆ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. By way of example only, “C₁-C₅ alkyl” indicates that there are one to five carbon atoms in the alkyl chain, i.e., the alkyl chain is selected from methyl, ethyl, n-propyl, isopropyl, n-butyl, isobutyl, tert-butyl, pentyl (branched and straight-chained), etc. Typical alkyl groups include, but are in no way limited to, methyl (“Me” or —CH₃), ethyl, propyl, isopropyl, butyl, isobutyl, tertiary butyl, pentyl, hexyl, and the like. An alkyl group may be unsubstituted or substituted.

As used herein, “alkenyl” refers to an alkyl group that contains in the straight or branched hydrocarbon chain one or more double bonds. As noted in the definition of “alkyl”, an alkenyl group may be unsubstituted or substituted.

As used herein, “alkynyl” refers to an alkyl group that contains in the straight or branched hydrocarbon chain one or more triple bonds. As noted in the definition of “alkyl”, an alkynyl group may be unsubstituted or substituted.

As used herein, the term “alkylene” refers to a bivalent fully saturated straight chain aliphatic hydrocarbon group. Examples of alkylene groups include, but are not limited to, methylene, ethylene, propylene, butylene, pentylene, hexylene, heptylene and octylene. An alkylene group may be represented by [symbol] , followed by the number of carbon atoms, followed by a “*”. For example,

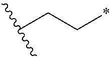 [SMILES: *CC*]

to represent ethylene. The alkylene group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkylene” where no numerical range is designated). The alkylene group may also be a medium size alkyl having 1 to 12 carbon atoms. The alkylene group could also be a lower alkyl having 1 to 6 carbon atoms. For example, a lower alkylene group can be substituted by replacing one or more hydrogen of the lower alkylene group and/or by substituting both hydrogens on the same carbon with a C₃₋₆ monocyclic cycloalkyl group

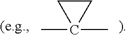

It also is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.” An alkylene group may be substituted or unsubstituted.

The term “halogen” or “halo,” as used herein, means any one of the radio-stable atoms of column 7 of the Periodic Table of the Elements, e.g., fluorine (—F), chlorine (—Cl), bromine (—Br), or iodine (—I).

As used herein, “haloalkyl” refers to a straight- or branched-chain alkyl group, substituting one or more hydrogens with halogens. Examples of haloalkyl groups include, but are not limited to, —CF₃, —CHF₂, —CH₂F, —CH₂CF₃, —CH₂CHF₂, —CH₂CH₂F, —CH₂CH₂Cl, —CH₂CF₂CF₃ and other groups that in light of the ordinary skill in the art and the teachings provided herein, would be considered equivalent to any one of the foregoing examples. The haloalkyl may be a medium sized or lower haloalkyl. An haloalkyl group may be substituted or unsubstituted.

As used herein, “alkoxy” refers to the formula —OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like. R may also be a carbocyclyl group. An alkoxy group may be substituted or unsubstituted.

As used herein, “polyethylene glycol” refers to the formula

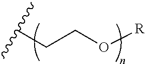

wherein n is an integer greater than one and R is a hydrogen or alkyl. The number of repeat units “n” may be indicated by referring to a number of members. Thus, for example, “2- to 5-membered polyethylene glycol” refers to n being an integer selected from two to five. In some embodiments, R is selected from methoxy, ethoxy, n-propoxy, 1-methylethoxy (isopropoxy), n-butoxy, iso-butoxy, sec-butoxy, and tert-butoxy.

As used herein, “heteroalkyl” refers to a straight or branched hydrocarbon chain (e.g., alkyl) containing one or more heteroatoms. A heteroatom is given its plain and ordinary meaning in organic chemistry, which includes an element other than carbon, including but not limited to, nitrogen (e.g., amino, etc.), oxygen (e.g., alkoxy, ether, hydroxyl, etc.), sulfur, and halogens. The heteroalkyl group may have 1 to 20 carbon atoms although the present definition also covers the occurrence of the term “heteroalkyl” where no numerical range is designated. The heteroalkyl group may also be a medium size heteroalkyl having 1 to 12 carbon atoms. The heteroalkyl group could also be a lower heteroalkyl having 1 to 6 carbon atoms. In various embodiments, the heteroalkyl may have from 1 to 4 heteroatoms, from 1 to 3 heteroatoms, 1 or 2 heteroatoms, or 1 heteroatom. The heteroalkyl group of the compounds may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain. A heteroalkyl group may be substituted or unsubstituted.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. For example, the aryl group can be a C₆-C₁₄ aryl group, a C₆-C₁₀ aryl group, or a C₆ aryl group. Examples of aryl groups include, but are not limited to, phenyl, naphthyl, azulenyl, and anthracenyl. An aryl group may be substituted or unsubstituted.

As used herein, “aryloxy” and “arylthio” refers to RO— and RS—, in which R is an aryl as is defined above, such as “C₆₋₁₀ aryloxy” or “C₆₋₁₀ arylthio” and the like, including but not limited to phenyloxy. An aryloxy or arylthio group may be substituted or unsubstituted.

An “aralkyl” or “arylalkyl” is an aryl group connected, as a substituent, via an alkylene group, such “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₆ alkylene group). An aralkyl or arylalkyl group may be substituted or unsubstituted.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system can be aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. For example, the heteroaryl group can contain 4 to 14 ring members (atoms in the ring(s)), 5 to 10 ring members (atoms in the ring(s)), 5 to 7 ring members (atoms in the ring(s)), 5 to 6 ring members (atoms in the ring(s)). The heteroaryl group may be designated as “5-7 membered heteroaryl,” “5-10 membered heteroaryl,” or similar designations. In various embodiments, a heteroaryl contains from 1 to 4 heteroatoms, from 1 to 3 heteroatoms, from 1 to 2 heteroatoms, or 1 heteroatom. For example, in various embodiments, a heteroaryl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. Examples of heteroaryl rings include, but are not limited to, furan (e.g., furyl), furazan (e.g., furazanyl), thiophene (e.g., thienyl), benzothiophene (e.g., benzothienyl), phthalazine (e.g., phthalazinyl), pyrrole (e.g., pyrrolyl), oxazole (e.g., oxazolyl), benzoxazole (e.g, benzoxazolyl), 1,2,3-oxadiazole, 1,2,4-oxadiazole, thiazole (e.g., thiazolyl), 1,2,3-thiadiazole, 1,2,4-thiadiazole, benzothiazole (e.g., benzothiazolyl), imidazole (e.g., imidazolyl), benzimidazole (e.g., benzimidazolyl), indole (e.g., indolyl), isoindole (e.g., isoindolyl), indazole, pyrazole (e.g., pyrazolyl), benzopyrazole, isoxazole (e.g., isoxazolyl), benzoisoxazole, isothiazole (e.g., isothiazolyl), triazole (e.g., triazolyl), benzotriazole, thiadiazole (e.g., thiadiazolyl), tetrazole, pyridine (e.g., pyridinyl), pyridazine (e.g., pyridazinyl), pyrimidine (e.g., pyrimidinyl), pyrazine (e.g., pyrazinyl), purine, pteridine, quinoline (e.g., quinolinyl), isoquinoline (e.g., isoquinlinyl), quinazoline, quinoxaline, cinnoline, and triazine (e.g., triazinyl). Heteroaryl rings may also include bridge head nitrogen atoms. For example but not limited to: pyrazolo[1,5-a]pyridine, imidazo[1,2-a]pyridine, and pyrazolo[1,5-a]pyrimidine. A heteroaryl group may be substituted or unsubstituted.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. A heteroaralkyl group may be substituted or unsubstituted.

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl. A carbocyclyl group may be substituted or unsubstituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s) or 3 to 8 atoms in the ring(s), or as otherwise noted herein. A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl.

A “(carbocyclyl)alkyl” is a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkenyl” means a carbocyclyl ring or ring system having at least one double bond, wherein no ring in the ring system is aromatic. An example is cyclohexenyl. cycloalkenyl groups can contain 4 to 10 atoms in the ring(s). A cycloalkenyl group may be substituted or unsubstituted.

As used herein, “heterocyclyl” or “heteroalicyclyl” refers to three-, four-, five-, six-, seven-, eight-, nine-, ten-, eleven-, twelve-, thirteen-, up to 20-membered monocyclic, bicyclic, and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) may be present in either a non-aromatic or aromatic ring in the ring system. The heteroatom(s) is an element other than carbon including, but not limited to, oxygen, sulfur, and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused fashion. Additionally, any nitrogens in a heteroalicyclic may be quaternized. Heterocyclyl or heteroalicyclic groups may be unsubstituted or substituted. Examples of such “heterocyclyl” or “heteroalicyclyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone, and their benzo-fused analogs (e.g., benzimidazolidinone, tetrahydroquinoline, 3,4-methylenedioxyphenyl). The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. A heterocyclyl group may be substituted or unsubstituted.

In various embodiments, a heterocyclyl contains from 1 to 4 heteroatoms, from 1 to 3 heteroatoms, from 1 to 2 heteroatoms, or 1 heteroatom. For example, in various embodiments, a heterocyclyl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline. A sulfur of the heterocyclyl ring may be provided as a dioxide (e.g., —S(O)₂—).

A “(heterocyclyl)alkyl” is a heterocyclyl group connected, as a substituent, via an alkylene group. Examples include, but are not limited to, imidazolinylmethyl and indolinylethyl.

A “(heterocyclyl)alkynyl” is a heterocyclyl group connected, as a substituent, via an alkynylene group.

As used herein, “acyl” refers to —C(═O)R, wherein R is hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. Non-limiting examples include formyl, acetyl, propanoyl, benzoyl, and acryl. An acyl group may be substituted or unsubstituted.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. An O-carboxy can be substituted or unsubstituted.

A “C-carboxy” group (or “ester”) refers to a “—C(═O)OR” group in which R is selected from hydrogen, —NH₂, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. A non-limiting example includes carboxyl (i.e., —C(═O)OH). A C-carboxy can be substituted or unsubstituted.

As used herein, the term “hydroxy” refers to a —OH group.

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. A sulfinyl can be substituted or unsubstituted.

A “sulfonyl” group refers to an “—SO₂R” or “—SO₂—” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. A sulfonyl can be provided in a heterocyclyl ring. A sulfonyl can be substituted or unsubstituted.

An “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. R_{A} and R_{B} may be taken together to provide a heteroaryl or heterocycle. A S-sulfonamido can be substituted or unsubstituted.

An “N-sulfonamido” group refers to a “—N(R_{A})SO₂R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. An N-sulfonamido can be substituted or unsubstituted.

An “O-carbamyl” group refers to a “—OC(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. R_{A} and R_{B} may be taken together to provide a heteroaryl or heterocycle. An O-carbamyl can be substituted or unsubstituted.

An “N-carbamyl” group refers to an “—N(R_{A})OC(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. An N-carbamyl can be substituted or unsubstituted.

An “O-thiocarbamyl” group refers to a “—OC(═S)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. R_{A} and R_{B} may be taken together to provide a heteroaryl or heterocycle. An O-thiocarbamyl can be substituted or unsubstituted.

An “N-thiocarbamyl” group refers to an “—N(R_{A})OC(═S)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. An N-thiocarbamyl can be substituted or unsubstituted.

A “C-amido” group refers to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. R_{A} and R_{B} may be taken together to provide a heteroaryl or heterocycle. A C-amido can be substituted or unsubstituted.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. R_{A} and R_{B} may be taken together to provide a heteroaryl or heterocycle. An N-amido can be substituted or unsubstituted.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. R_{A} and R_{B} may be taken together to provide a heteroaryl or heterocycle. An amino can be substituted or unsubstituted.

An “alkamino” group refers to a “—NR_{A}R_{B}” group in which R_{A} is C₁₋₆ alkylene (e.g., connected to an indicated structural substituent) or C₁₋₆ alkyl and R_{B} is independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. An alkamino can be substituted or unsubstituted.

An “aminoalkyl” group refers to an amino group connected via an alkylene group. An aminoalkyl can be substituted or unsubstituted.

A “carbamido” or “carbamide” group refers to a “(R_{A}R_{B})NC(═O)N(R_{C})—” group in which R_{A}, R_{B}, and R_{C} can be independently hydrogen, alkyl, alkenyl, alkynyl, carbocyclyl, aryl, heteroaryl, heterocyclyl, aralkyl, or heterocyclyl(alkyl), as defined herein. A carbamido may be substituted or unsubstituted.

An “alkoxyalkyl” group refers to an alkoxy group connected via an alkylene group, such as a “C₂₋₈ alkoxyalkyl” and the like. An alkoxyalkyl can be substituted or unsubstituted.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl (optionally substituted with —OH or C-carboxy), C₁-C₆ alkenyl, C₁-C₆ alkynyl, C₁-C₆ heteroalkyl, C₃-C₇ carbocyclyl (optionally substituted with halo, —OH, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with N-amido, —OH, halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), haloalkoxy, cycloalkenyl, halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, a mono-substituted amine group, a di-substituted amine group, a mono-substituted amine(alkyl), a di-substituted amine(alkyl), nitro, thiocarbonyl, 0-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfenyl, sulfinyl, sulfonyl, —O—NH₂, oxo (═O), a diamino-group, a polyamino, a diether-group, and a polyether (e.g., diethylene glycol, triethylene glycol, oligoethylene glycol, polyethylene glycol, etc.). Wherever a group is described as “optionally substituted” (or other similar language) or as comprising one or more “optional substitutions,” that group can be substituted with the above substituents or can be unsubstituted.

In some embodiments, substituted group(s) is (are) substituted with one or more substituent(s) individually and independently selected from C₁-C₄ alkyl, amino, hydroxy, and halogen.

Two substituents may come together with the atom or atoms to which they are attached to form a ring that is spiro or fused with the rest of the compound.

As used herein, any “R” group(s) such as, without limitation, R¹, R², R³, etc., represent substituents that can be attached to the indicated atom. An R group may be substituted or unsubstituted. If two “R” groups are described as being “taken together” (or similar language), the R groups and the atoms they are attached to can form a cycloalkyl, aryl, heteroaryl or heterocycle. When two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

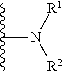

and R¹ and R² are defined as selected from the group consisting of hydrogen and alkyl, or R¹ and R² together with the nitrogen to which they are attached form a heterocyclyl, it is meant that R¹ and R² can be selected from hydrogen or alkyl, or alternatively, the substructure has structure:

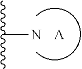

where ring A is a heterocyclyl ring containing the depicted nitrogen. As further illustration, without limitation, if R^{1a} and R^{1b} of an NR^{1a}R^{1b} group are indicated to be “taken together,” it means that they are covalently bonded to one another to form a ring:

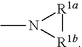

A cyclic structure may be shown using provided using the following structure (or a similar structure with a different ring size, heteroatoms, etc.):

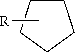

When a cyclic structure is illustrated using this type of structure, what is meant is that the R group may be attached to any position of the ring by replacing an —H of the ring with —R. For example, for the following ring:

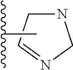

includes any of the following ring structures:

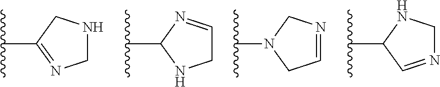

where

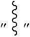

indicates a bond to a remaining portion of the structure. Likewise, the following structure:

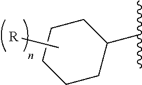

where

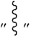

indicates a bond to a remaining portion of the structure and n is 1 to 5, any of the following structures are envisioned or other variations (as would be readily appreciated by the one of skill in the art):

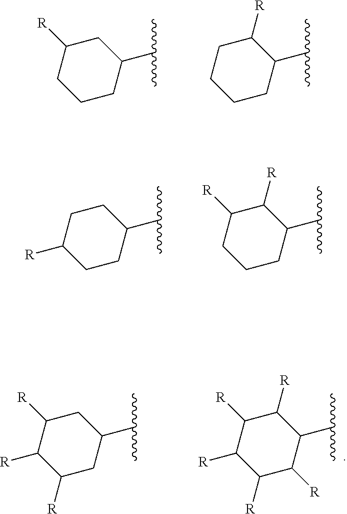

When two “adjacent” R groups are said to form a ring “together with the atoms to which they are attached,” it is meant that the collective unit of the atoms, intervening bonds, and the two R groups are the recited ring. For example, when the following substructure is present:

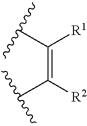

and R¹ and R² are defined as selected from the group consisting of hydrogen and alkyl, or R¹ and R² together with the atoms to which they are attached form an aryl or carbocyclyl, it is meant that R¹ and R² can be selected from hydrogen or alkyl, or alternatively, the substructure has structure:

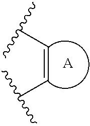

where A is an aryl ring or a carbocyclyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

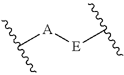

includes the substituent being oriented such that the “A” is attached at the leftmost attachment point of the molecule as well as the case in which “A” is attached at the rightmost attachment point of the molecule.

As noted in the definition for alkylene, it also is to be understood that certain radical naming conventions can include either a mono-radical or a di-radical, depending on the context. For example, where a substituent (e.g., in a genus structure) requires two points of attachment to the rest of the molecule, it is understood that the substituent is a di-radical. For example, a substituent identified as alkamino that requires two points of attachment includes di-radicals such as —NHCH₂—, —NHCH₂CH₂—, —NHCH₂CH(CH₃)CH₂—, and the like. Other examples a substituent may require two points of attachment include alkoxy, aryl, heteroaryl, carbocyclyl, heterocyclyl, etc.

As used herein, a radical indicates species with a single, unpaired electron such that the species containing the radical can be covalently bonded to another species. Hence, in this context, a radical is not necessarily a free radical. Rather, a radical indicates a specific portion of a larger molecule.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition it is understood that, in any compound described herein having one or more double bond(s) generating geometrical isomers that can be defined as E or Z, each double bond may independently be E or Z a mixture thereof. It is understood that, in any compound described herein having one or more chiral centers, all possible diastereomers are also envisioned. It is understood that, in any compound described herein all tautomers are envisioned. It is also understood that, in any compound described herein, all isotopes of the included atoms are envisioned. For example, any instance of hydrogen, may include hydrogen-1 (protium), hydrogen-2 (deuterium), hydrogen-3 (tritium) or other isotopes; any instance of carbon may include carbon-12, carbon-13, carbon-14, or other isotopes; any instance of oxygen may include oxygen-16, oxygen-17, oxygen-18, or other isotopes; any instance of fluorine may include one or more of fluorine-18, fluorine-19, or other isotopes; any instance of sulfur may include one or more of sulfur-32, sulfur-34, sulfur-35, sulfur-36, or other isotopes.

As used herein, the term “inhibitor” means any compound, molecule or composition that inhibits or reduces the activity of a target biomolecule. The inhibition can be achieved by, for example, blocking phosphorylation of the target (e.g., competing with adenosine triphosphate (ATP), a phosphorylating entity), by binding to a site outside the active site, affecting its activity by a conformational change, or by depriving kinases of access to the molecular chaperoning systems on which they depend for their cellular stability, leading to their ubiquitylation and degradation.

As used herein, “subject,” “host,” “patient,” and “individual” are used interchangeably and shall be given its ordinary meaning and shall also refer to an organism that has JMJD3 proteins. This includes mammals, e.g., a human, a non-human primate, ungulates, canines, felines, equines, mice, rats, and the like. The term “mammal” includes both human and non-human mammals.

“Diagnosis” as used herein shall be given its ordinary meaning and shall also include determination of a subject's susceptibility to a disease or disorder, determination as to whether a subject is presently affected by a disease or disorder, prognosis of a subject affected by a disease or disorder (e.g., identification of cancer or cancerous states, stages of cancer, or responsiveness of cancer to therapy), and use of therametrics (e.g., monitoring a subject's condition to provide information as to the effect or efficacy of therapy).

The term “sample” or “biological sample” shall be given its ordinary meaning and also encompasses a variety of sample types obtained from an organism and can be used in an imaging, a diagnostic, a prognostic, or a monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

As used herein, a “natural amino acid side chain” refers to the side-chain substituent of a naturally occurring amino acid. Naturally occurring amino acids have a substituent attached to the α-carbon. Naturally occurring amino acids include Arginine, Lysine, Aspartic acid, Glutamic acid, Glutamine, Asparagine, Histidine, Serine, Threonine, Tyrosine, Cysteine, Methionine, Tryptophan, Alanine, Isoleucine, Leucine, Phenylalanine, Valine, Proline, and Glycine.

As used herein, a “non-natural amino acid side chain” refers to the side-chain substituent of a non-naturally occurring amino acid. Non-natural amino acids include β-amino acids (β³ and β²), Homo-amino acids, Proline and Pyruvic acid derivatives, 3-substituted Alanine derivatives, Glycine derivatives, Ring-substituted Phenylalanine and Tyrosine Derivatives, Linear core amino acids and N-methyl amino acids. Exemplary non-natural amino acids are available from Sigma-Aldridge, listed under “unnatural amino acids & derivatives.” See also, Travis S. Young and Peter G. Schultz, “Beyond the Canonical 20 Amino Acids: Expanding the Genetic Lexicon,” J. Biol. Chem. 2010 285: 11039-11044, which is incorporated by reference in its entirety.

The term “agent” or “test agent” includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances. Unless otherwise specified, the terms “agent”, “substance”, and “compound” are used interchangeably herein.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved characteristics (such as higher binding affinity for a target molecule) is an approach that is well known in pharmaceutical chemistry.

The “patient” or “subject” treated as disclosed herein is, in some embodiments, a human patient, although it is to be understood that the principles of the presently disclosed subject matter indicate that the presently disclosed subject matter is effective with respect to all vertebrate species, including mammals, which are intended to be included in the terms “subject” and “patient.” Suitable subjects are generally mammalian subjects. The subject matter described herein finds use in research as well as veterinary and medical applications. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rats and mice but also includes many other species.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

An “effective amount” or a “therapeutically effective amount” as used herein refers to an amount of a therapeutic agent that is effective to relieve, to some extent, or to reduce the likelihood of onset of, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

“Treat,” “treatment,” or “treating,” as used herein refers to administering a pharmaceutical composition for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject.

As used herein, the term “weight percent,” when referring to a component, is the weight of the component divided by the weight of the composition that includes the component, multiplied by 100%. For example, the weight percent of component A when 5 grams of component A is added to 95 grams of component B is 5% (e.g., 5 g A/(5 g A+95 g B)×100%).

The term “control” refers shall be given its ordinary meaning and shall also include a sample or standard used for comparison with a sample which is being examined, processed, characterized, analyzed, etc. In several embodiments, the control is a sample obtained from a healthy patient or a non-tumor tissue sample obtained from a patient diagnosed with a tumor. In several embodiments, the control is a historical control or standard reference value or range of values. In several embodiments, the control is a comparison to a wild-type JMJD3 arrangement or scenario.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “comprising” as used herein is synonymous with “including,” “containing,” or “characterized by,” and is inclusive or open-ended and does not exclude additional, unrecited elements or method steps; the term “having” should be interpreted as “having at least;” the term “includes” should be interpreted as “includes but is not limited to;” the term “example” is used to provide exemplary instances of the item in discussion, not an exhaustive or limiting list thereof; and use of terms like “preferably,” “preferred,” “desired,” or “desirable,” and words of similar meaning should not be understood as implying that certain features are critical, essential, or even important to the structure or function of the invention, but instead as merely intended to highlight alternative or additional features that may or may not be utilized in a particular embodiment of the invention. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Additionally, the phrase “consisting essentially of” will be understood to include those elements specifically recited and those additional elements that do not materially affect the basic and novel characteristics of the claimed technology. The phrase “consisting of” excludes any element not specified.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

The section headings used herein are for organizational purposes only and are not to be construed as limiting the subject matter described. Features disclosed under one heading (such as a composition) can be used in combination with features disclosed under a different heading (a method of treating).

### INTRODUCTION

Jumonji protein activity (e.g., JMJD3) is associated with several diseases, including autoimmune diseases, including Ulcerative colitis, Crohn's Disease, Rheumatoid Arthritis, Systemic Lupus Erythematosus, Atopic Dermatitis, Psoriasis, Type 1 Diabetes, Graft-versus-host-disease (GVHD), Autoimmune Encephalitis, Bechet's Disease and Sjogren's Syndrome. Furthermore, the effects of the JMJD3 inhibitors against IL5 and IL13 suggest their use in asthma, lung allergies and also atopic dermatitis. Small molecule inhibitors of Jumonji protein activity (e.g., JMJD3), have been used to treat inflammatory and autoimmune disease. However, despite the fact that various inhibitors of JMJD3 are known, there remains a need for selective inhibitors to be used for the treatment of diseases which offer one or more advantages over current compounds. Those advantages include: improved activity and/or efficacy; beneficial target selectivity profile according to the respective therapeutic need; improved side effect profile, such as fewer undesired side effects, lower intensity of side effects; improved targeting of mutant receptors in diseased cells; improved physicochemical properties, such as solubility/stability in water, body fluids, and/or pharmaceutical formulations; improved pharmacokinetic properties, allowing e.g. for dose reduction or an easier dosing scheme; easier drug substance manufacturing e.g. by shorter synthetic routes or easier purification. Several embodiments disclosed herein pertain to compounds that achieve one or more of these advantages (or others). Several embodiments disclosed herein pertain to compounds that address one or more deficiencies of known drug substances.

### Formula I Genus and Compounds

Several embodiments pertain to substituted triazine compounds (e.g., pyrazole-triazines). Triazine is a molecule having the following numbering convention:

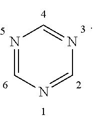

In several embodiments, the substituted triazine is a compound having the structure of Formula (I) (or a pharmaceutically acceptable salt thereof):

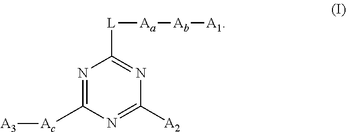

In several embodiments, the variables are as disclosed elsewhere herein.

In several embodiments, L is selected from the group consisting of —C(R¹)₂—NR¹—, —N(R¹)₂, optionally substituted amine(alkyl), —O—, —C(═O)—, —OC(═O)—, —C(═O)O—, and optionally substituted heterocyclyl, or L is not present. In several embodiments, where an intermediate variable (e.g., a variable that is between two other structural features) is described as optionally being “not present,” when not present the variable can be expressed as a single bond between two adjacent groups. For example, where L is not present, the triazine ring may be directly connected to the Aₐ shown in Formula (I). Likewise, where L and Aₐ are not present, the triazine ring may be directly connected to A_{b}. As will be appreciated by one in skill in the art, in several embodiments, the triazine ring of Formula (I) may bonded directly to L (where L is present), Aₐ (where L is not present but Aₐ is present), A_{b} (where L and Aₐ are not present but A_{b} is present), or A₁ (where L, Aₐ, and A_{b} are not present).

In several embodiments, Aₐ is selected from the group consisting of optionally substituted C₁-C₆ alkylene and optionally substituted C₁-C₆ alkyl. In several embodiments, Aₐ is not present. In several embodiments, Aₐ is not present.

In several embodiments, A_{b} is selected from the group consisting of —C(═O)-or —N(CH₃)—. In several embodiments, A_{b} is not present. In several embodiments, A_{b} is not present and A₁ is bonded directly to Aₐ or L.

In several embodiments, A₁ is selected from the group consisting of —H, hydroxyl, amino, optionally substituted C₁₋₁₂ alkyl, optionally substituted C₁-C₁₂ alkoxy, optionally substituted C₁-C₁₂ alkamino, optionally substituted carbamide, C-amido, optionally substituted C₆₋₁₀ aryl, optionally substituted 5-12 membered heteroaryl, optionally substituted C₃₋₁₀ carbocyclyl, and optionally substituted 3-12 membered heterocyclyl. In several embodiments, A₁ is not present.

In several embodiments, where a terminal variable (e.g., a variable providing a terminal end of a structure) is described as optionally being “not present,” when not present, the variable may be absent or, where required to complete the valency of an adjacent substituent, may be a hydrogen atom on the adjacent substituent. For example, if A₁, Aₐ, A_{b} are is not present and L is —N(R¹)₂, then L may be expressed as

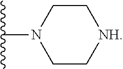 [SMILES: *N1CCNCC1]

As further illustration, if A₁ and A_{b} are is not present, and Aₐ is alkylene, then A₁ may be expressed as —H to provide a combination of Aₐ and A₁ as an alkyl. In several embodiments, A₁ is not present, L is not bonded to Aₐ or A_{b}, and L is bonded to other groups as described elsewhere herein, (e.g., Formula (Id)).

In several embodiments, A₂ is selected from the group consisting of optionally substituted C₆₋₁₀ aryl, optionally substituted 5-12 membered heteroaryl, and optionally substituted 3-12 membered heterocyclyl.

In several embodiments, A_{c} is selected from the group consisting of optionally substituted C₁₋₃ alkylene, optionally substituted C₁-C₃ alkamino, —O—, —NR¹—, —N(R¹)₂, and a single bond. In several embodiments, A_{c} is not present and A₃ is bonded directly to the triazine ring of Formula (I).

In several embodiments, A₃ is selected from the group consisting of optionally substituted C₁₋₁₂ alkyl, optionally substituted C₁-C₁₂ alkoxy, optionally substituted C₁-C₁₂ alkamino, optionally substituted C₆₋₁₀ aryl, optionally substituted 5-12 membered heteroaryl, optionally substituted C₃₋₁₀ carbocyclyl, and optionally substituted 3-12 membered heterocyclyl.

In several embodiments, each instance of R¹, where present, is independently selected from the group consisting of —H, halogen, hydroxy, optionally substituted C₁₋₆ alkyl, optionally substituted C₁-C₆ alkoxy, optionally substituted C₁₋₆ haloalkyl, and optionally substituted C₆₋₁₀ aryl. In several embodiments, where two R¹ groups are present, the R¹ groups taken together form an optionally substituted 3-12 membered heterocycle or an optionally substituted C₃₋₁₀ carbocyclyl. Where more than one R¹ is present, those R¹ variables may be the same or different.

In several embodiments, any one of the substituents of Aₐ, A_{b}, A_{c}, A₁, A₂, A₃, or R¹ (or any other variable disclosed herein) may be optionally substituted. In several embodiments, when any one of Aₐ, A_{b}, A_{c}, A₁, A₂, A₃, or R¹ (or any other variable disclosed herein) is substituted with one or more optional substitutions, each of the one or more optional substitutions are independently selected from the optionally substituents disclosed in the Definitions section. In several embodiments, when any one of Aₐ, A_{b}, A_{c}, A₁, A₂, A₃, or R¹ (or any other variable disclosed herein) is substituted with one or more optional substitutions, each of the one or more optional substitutions are independently selected from the group consisting of —OH, C₁₋₃ alkyl, C₁₋₃ alkoxy, or halogen. In several embodiments, when any one of Aₐ, A_{b}, A₁, A₂, A₃, or R¹ (or any other variable disclosed herein) is substituted with one or more optional substitutions, each of the one or more optional substitutions are independently selected from the group consisting of -Me or —F. In several embodiments, when the A₁ group is substituted with one or more optional substitutions, the one or more optional substitutions are independently selected from the group consisting of acyl, C₁₋₆ alkyl, C₁₋₆ alkoxy, C-amido, aryl, ester, halogen, heteroaryl, heterocyclyl (including providing a spiro configuration where A₁ is a heterocyclyl or carbocyclyl group), and hydroxy. In several embodiments, when the A₃ group is substituted with one or more optional substitutions, the one or more optional substitutions are independently selected from the group consisting of C₁₋₆ alkyl, C₁₋₆ cycloalkyl, C₆₋₁₀ aryl, halogen, and hydroxy.

In several embodiments, L is —N(R¹)₂. In several embodiments, L is —N(R¹)₂ and the R¹ groups taken together form an optionally substituted 3-12 membered heterocyclyl group. In several embodiments, L is substituted with a heterocyclyl group. In several embodiments, L is a 6-membered heterocyclyl group comprising 1 or 2 heteroatoms. In several embodiments, the heteroatoms of L are N. In several embodiments, L is substituted with a 4-6 membered heterocyclyl group. In several embodiments, where L is a heterocyclyl group substituted with an additional heterocyclyl group, the two rings provide a spiro conformation. In several embodiments, L is substituted with four membered heterocyclyl group. In several embodiments, Aₐ, A_{b}, and A₁ are not present and L is 2-oxa-5,8-diazaspiro[3.5]nonane.

In several embodiments, L is —N(R¹)₂ and the R¹ groups taken together form an optionally substituted 3-12 membered heterocyclyl group. In several embodiments, Aₐ and A_{b} are not present. In several embodiments, A₁ is a 3-12 membered heterocyclyl group (e.g., 3 to 6 membered heterocyclyl, 3 to 5 membered heterocyclyl, 4-membered heterocyclyl, etc.) and the ring of A₁ and the ring of L provide a spiro ring system.

In several embodiments, L is —N(R¹)₂ and the R¹ groups taken together form an optionally substituted 3-12 membered heterocyclyl group. In several embodiments, Aₐ and A_{b} are not present. In several embodiments, L, Aₐ, A_{b}, and A₁ together provide a structure selected from the group consisting of:

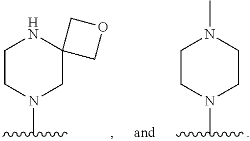

In several embodiments, L is —N(R¹)— or —NH—. In several embodiments, Aₐ is C₁-C₆ alkylene. In several embodiments, Aₐ is C₁-C₃ alkylene. In several embodiments, A_{b} is —C(═O)— or is not present. In several embodiments, A₁ is amino, hydroxyl, C₁₋₃ alkyl, carbamide optionally substituted with methyl or ethyl, 3 to 6 membered heterocyclyl, 3 to 6 membered heteroaryl, or C₁-C₃ alkoxy.

In several embodiments, L, Aₐ, A_{b}, and A₁ together provide a structure selected from the group consisting of:

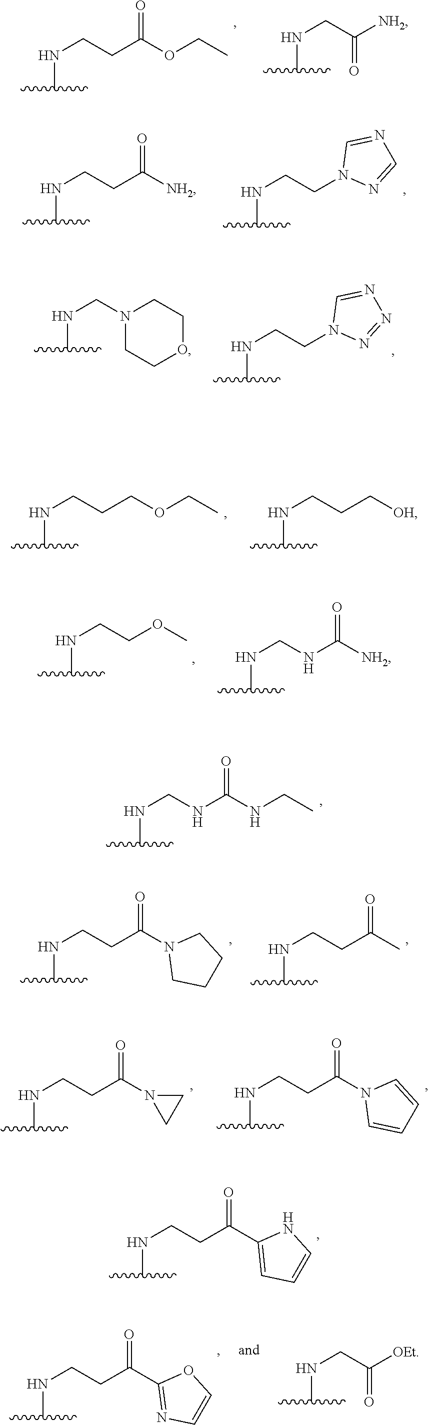

Any one of the directly preceding structures may be further optionally substituted by replacing one or more —H atoms of any carbon or nitrogen atom present within the structure.

In several embodiments, L is —N(R¹)— or —NH—. In several embodiments, Aₐ is not present. In several embodiments, A_{b} is not present. In several embodiments, A₁ is C₁₋₄ alkyl optionally substituted with hydroxyl or halogen, optionally substituted C₃₋₆ carbocyclyl, 3 to 6 membered heterocyclyl, 3 to 6 membered aryl optionally substituted with halogen, or optionally substituted 3 to 6 membered heteroaryl. In several embodiments, L, Aₐ, A_{b}, and A₁ together provide a structure selected from the group consisting of:

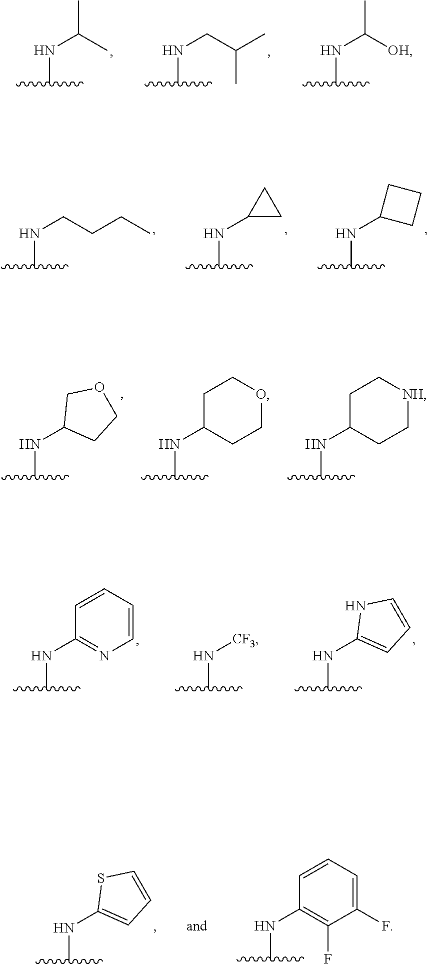

Any one of the directly preceding structures may be further optionally substituted by replacing one or more —H atoms of any carbon or nitrogen atom present within the structure.

In several embodiments, A₂ is selected from the group consisting of optionally substituted 5-6 membered heteroaryl or optionally substituted 5-6 membered heterocyclyl. In several embodiments, A² comprises 1, 2, or three heteroatoms. In several embodiments, the heteroatoms of A² are N and/or S. In several embodiments, A₂ is a structure selected from the group consisting of:

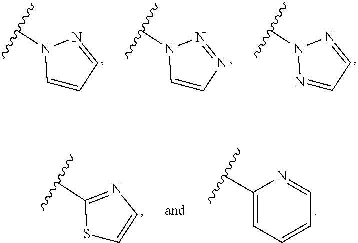 [SMILES: *n1cccn1 ; *n1ccnn1 ; *n1nccn1 ; *c1nccs1 ; *c1ccccn1]

Any one of the directly preceding structures may be further optionally substituted by replacing one or more —H atoms of any carbon or nitrogen atom present within the structure.

In several embodiments, A_{c} is —O— or is not present. In several embodiments, A₃ is selected from the group consisting of C₁₋₅ alkyl, phenyl optionally substituted with —F, C₁-C₃ alkamino, optionally substituted 5-12 membered heteroaryl, optionally substituted C₃₋₁₀ carbocyclyl, and optionally substituted 3-12 membered heterocyclyl.

In several embodiments, A_{c} and A₃ together provide a structure selected from the group consisting of

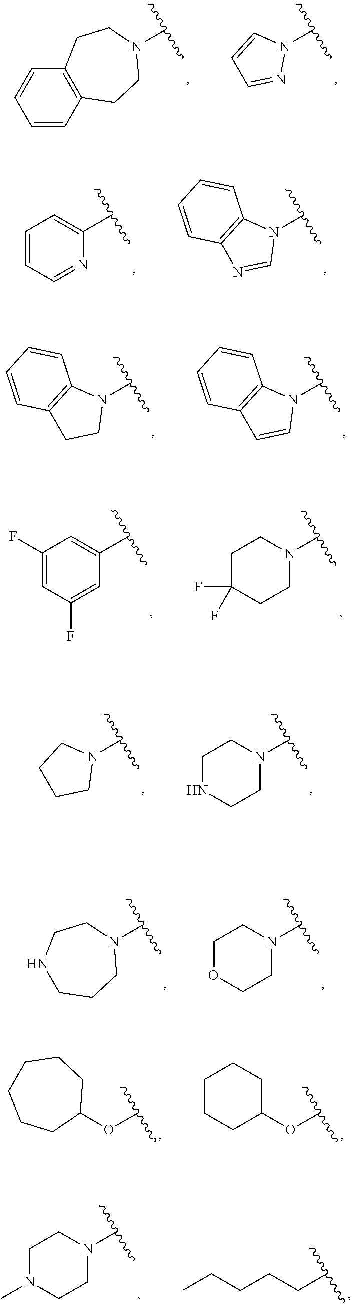

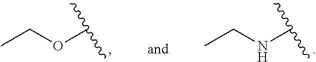

Any one of the directly preceding structures may be further optionally substituted by replacing one or more —H atoms of any carbon or nitrogen atom present within the structure.

In several embodiments, A_{c} is C₁₋₃ alkylene or C₁-C₃ alkamino. In several embodiments, A₃ is selected from the group consisting of phenyl optionally substituted with —F and optionally substituted C₃₋₇ carbocyclyl. In several embodiments, A_{c} and A₃ together provide a structure selected from the group consisting of

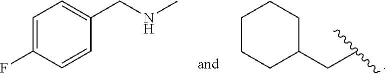

In several embodiments, Formula (I) may be represented by one or more of the following compounds (or others):

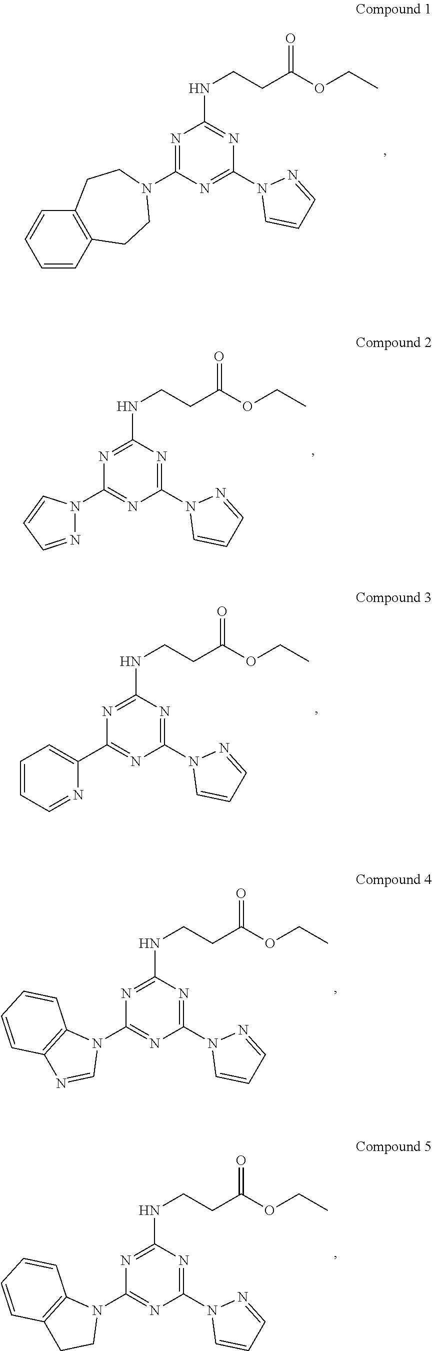 [SMILES: CCOC(=O)CCNc1nc(N2CCc3ccccc3CC2)nc(-n2cccn2)n1 ; CCOC(=O)CCNc1nc(-n2cccn2)nc(-n2cccn2)n1 ; CCOC(=O)CCNc1nc(-c2ccccn2)nc(-n2cccn2)n1 ; CCOC(=O)CCNc1nc(-n2cccn2)nc(-n2cnc3ccccc32)n1 ; CCOC(=O)CCNc1nc(N2CCc3ccccc32)nc(-n2cccn2)n1]

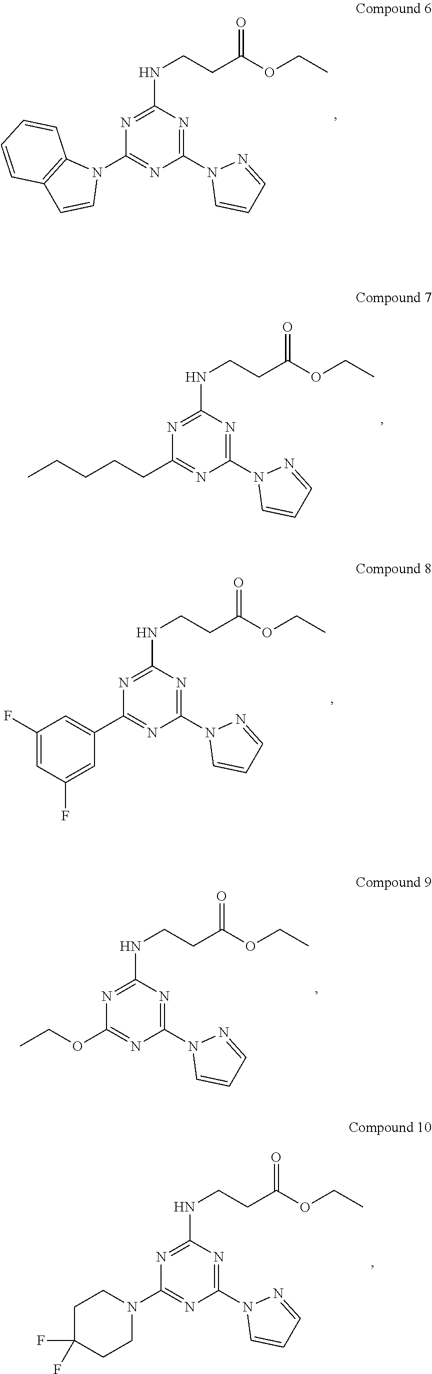

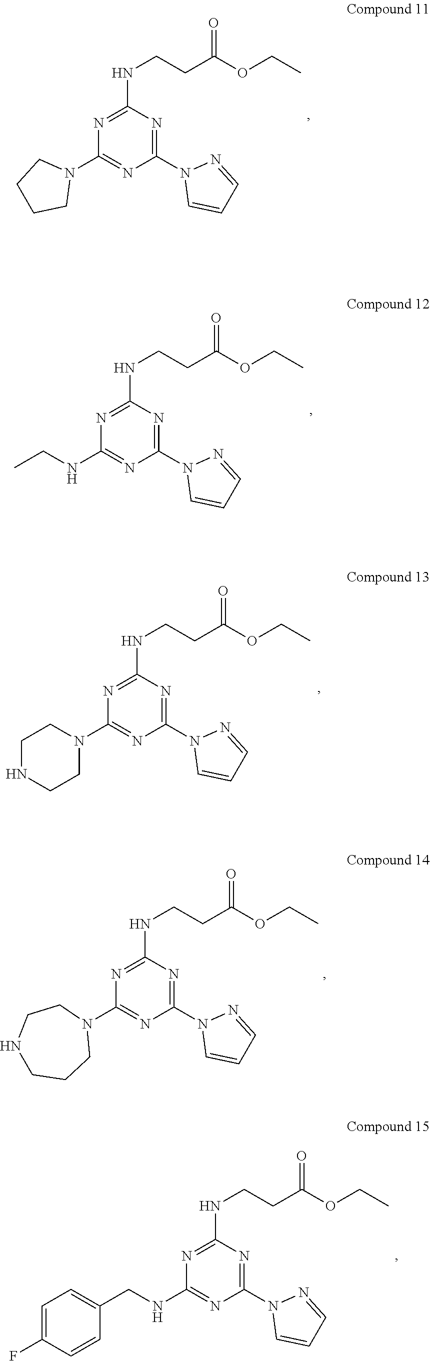

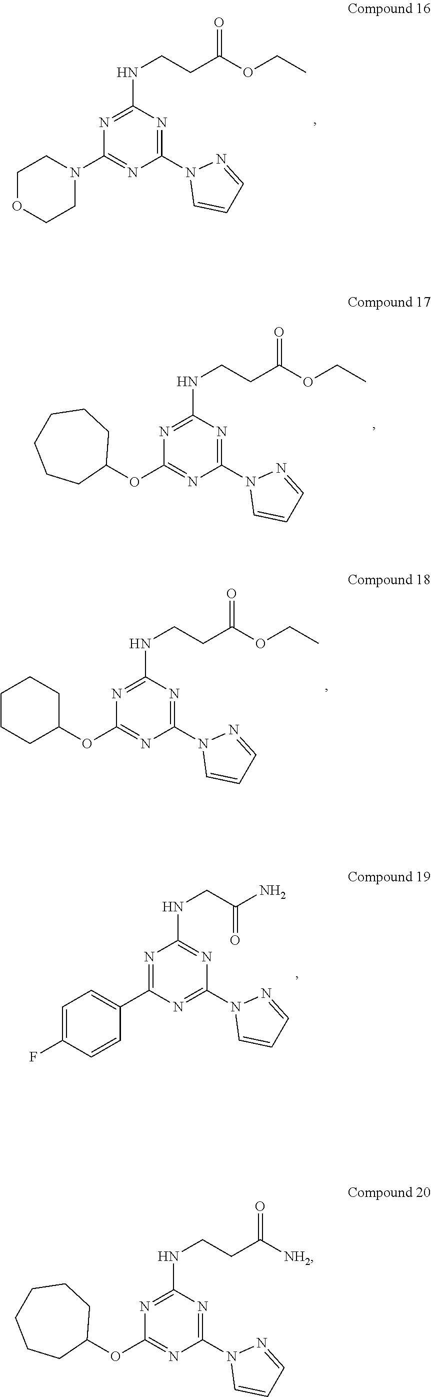 [SMILES: CCOC(=O)CCNc1nc(N2CCOCC2)nc(-n2cccn2)n1 ; CCOC(=O)CCNc1nc(OC2CCCCCC2)nc(-n2cccn2)n1 ; CCOC(=O)CCNc1nc(OC2CCCCC2)nc(-n2cccn2)n1 ; NC(=O)CNc1nc(-c2ccc(F)cc2)nc(-n2cccn2)n1 ; NC(=O)CCNc1nc(OC2CCCCCC2)nc(-n2cccn2)n1]

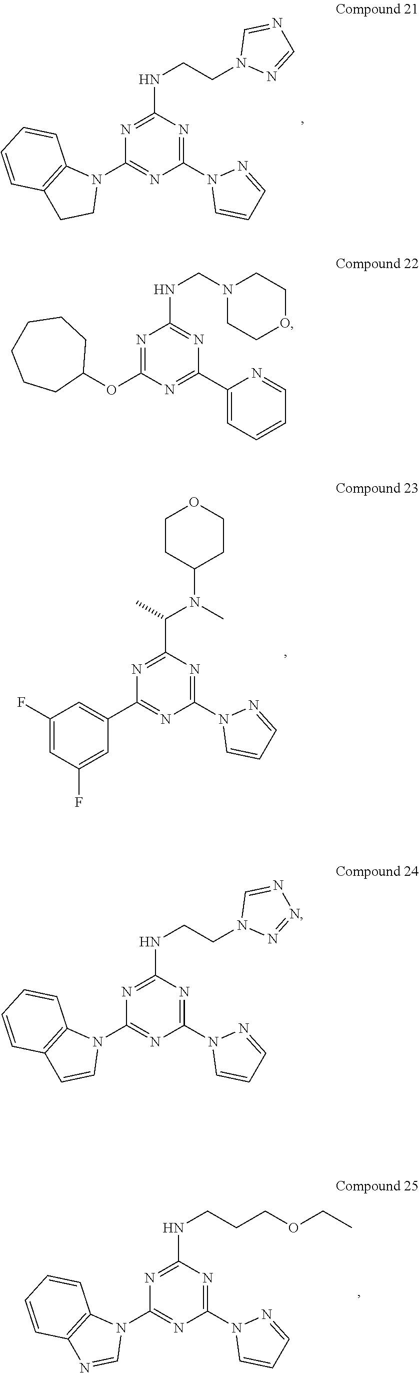 [SMILES: c1ccc2c(c1)CCN2c1nc(NCCn2cncn2)nc(-n2cccn2)n1 ; c1ccc(-c2nc(NCN3CCOCC3)nc(OC3CCCCCC3)n2)nc1 ; C[C@@H](c1nc(-c2cc(F)cc(F)c2)nc(-n2cccn2)n1)N(C)C1CCOCC1 ; c1ccc2c(c1)ccn2-c1nc(NCCn2cnnn2)nc(-n2cccn2)n1 ; CCOCCCNc1nc(-n2cccn2)nc(-n2cnc3ccccc32)n1]

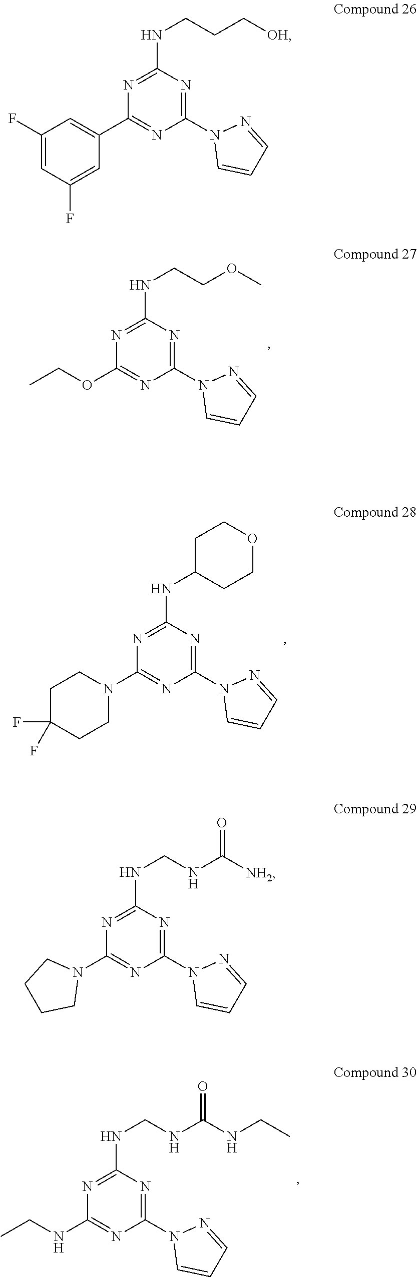

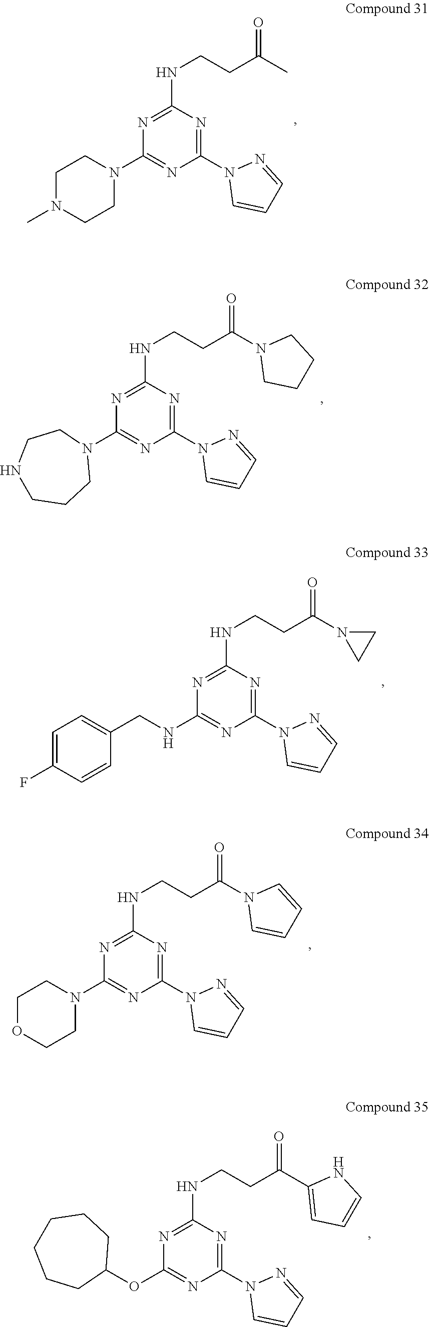 [SMILES: CC(=O)CCNc1nc(N2CCN(C)CC2)nc(-n2cccn2)n1 ; O=C(CCNc1nc(N2CCCNCC2)nc(-n2cccn2)n1)N1CCCC1 ; O=C(CCNc1nc(NCc2ccc(F)cc2)nc(-n2cccn2)n1)N1CC1 ; O=C(CCNc1nc(N2CCOCC2)nc(-n2cccn2)n1)n1cccc1 ; O=C(CCNc1nc(OC2CCCCCC2)nc(-n2cccn2)n1)c1ccc[nH]1]

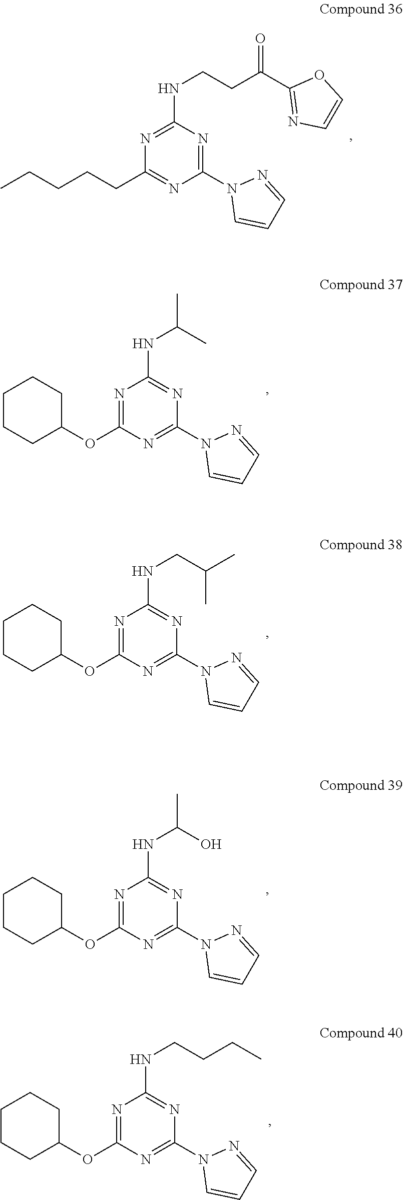 [SMILES: CCCCCc1nc(NCCC(=O)c2ncco2)nc(-n2cccn2)n1 ; CC(C)Nc1nc(OC2CCCCC2)nc(-n2cccn2)n1 ; CC(C)CNc1nc(OC2CCCCC2)nc(-n2cccn2)n1 ; CC(O)Nc1nc(OC2CCCCC2)nc(-n2cccn2)n1 ; CCCCNc1nc(OC2CCCCC2)nc(-n2cccn2)n1]

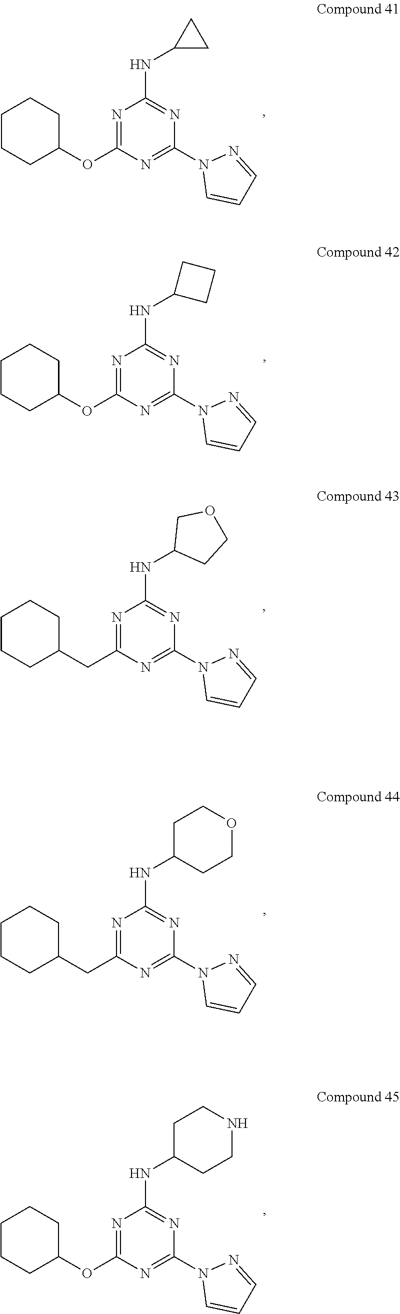 [SMILES: c1cnn(-c2nc(NC3CC3)nc(OC3CCCCC3)n2)c1 ; c1cnn(-c2nc(NC3CCC3)nc(OC3CCCCC3)n2)c1 ; c1cnn(-c2nc(CC3CCCCC3)nc(NC3CCOC3)n2)c1 ; c1cnn(-c2nc(CC3CCCCC3)nc(NC3CCOCC3)n2)c1 ; c1cnn(-c2nc(NC3CCNCC3)nc(OC3CCCCC3)n2)c1]

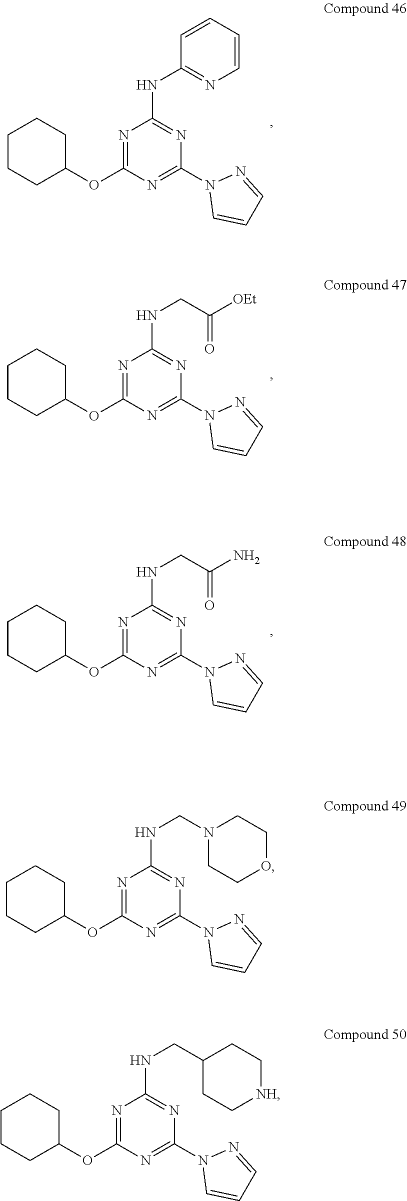 [SMILES: c1ccc(Nc2nc(OC3CCCCC3)nc(-n3cccn3)n2)nc1 ; CCOC(=O)CNc1nc(OC2CCCCC2)nc(-n2cccn2)n1 ; NC(=O)CNc1nc(OC2CCCCC2)nc(-n2cccn2)n1 ; c1cnn(-c2nc(NCN3CCOCC3)nc(OC3CCCCC3)n2)c1 ; c1cnn(-c2nc(NCC3CCNCC3)nc(OC3CCCCC3)n2)c1]

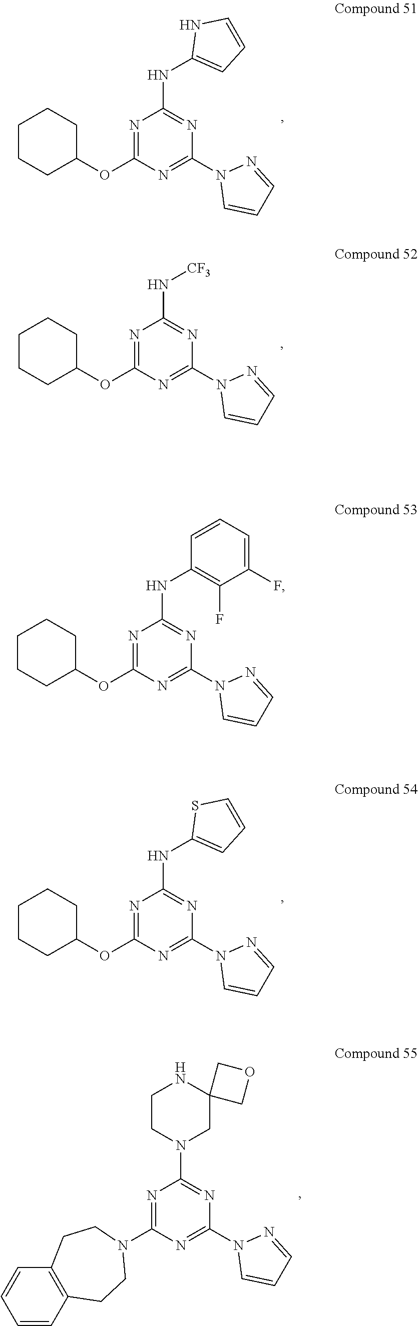 [SMILES: c1c[nH]c(Nc2nc(OC3CCCCC3)nc(-n3cccn3)n2)c1 ; FC(F)(F)Nc1nc(OC2CCCCC2)nc(-n2cccn2)n1 ; Fc1cccc(Nc2nc(OC3CCCCC3)nc(-n3cccn3)n2)c1F ; c1csc(Nc2nc(OC3CCCCC3)nc(-n3cccn3)n2)c1 ; c1ccc2c(c1)CCN(c1nc(N3CCNC4(COC4)C3)nc(-n3cccn3)n1)CC2]

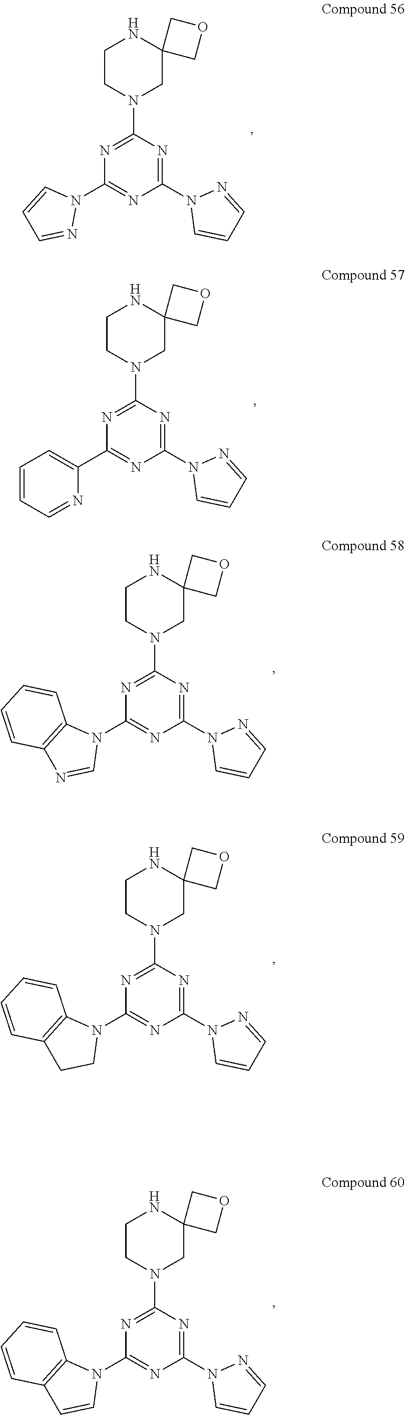 [SMILES: c1cnn(-c2nc(N3CCNC4(COC4)C3)nc(-n3cccn3)n2)c1 ; c1ccc(-c2nc(N3CCNC4(COC4)C3)nc(-n3cccn3)n2)nc1 ; c1ccc2c(c1)ncn2-c1nc(N2CCNC3(COC3)C2)nc(-n2cccn2)n1 ; c1ccc2c(c1)CCN2c1nc(N2CCNC3(COC3)C2)nc(-n2cccn2)n1 ; c1ccc2c(c1)ccn2-c1nc(N2CCNC3(COC3)C2)nc(-n2cccn2)n1]

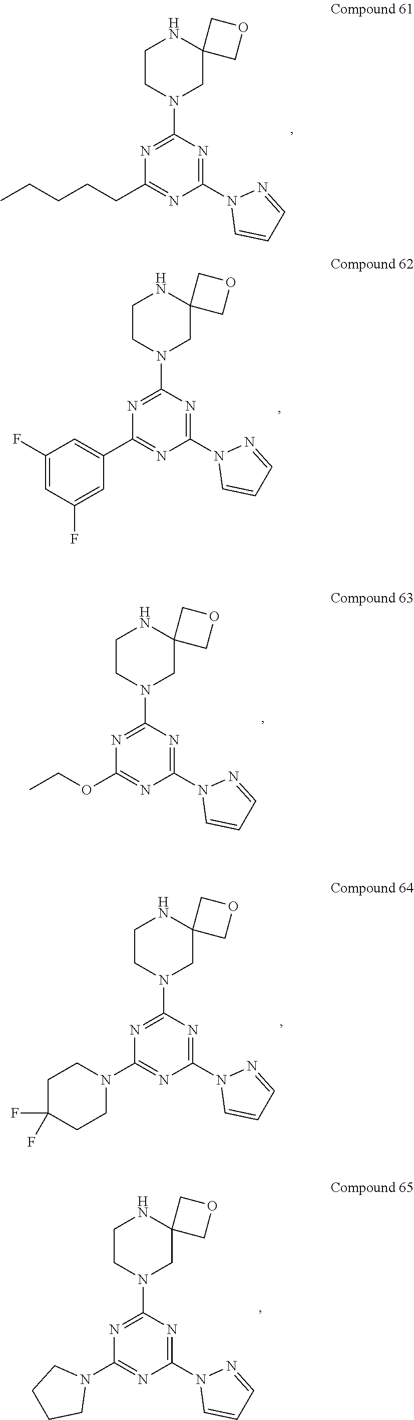 [SMILES: CCCCCc1nc(N2CCNC3(COC3)C2)nc(-n2cccn2)n1 ; Fc1cc(F)cc(-c2nc(N3CCNC4(COC4)C3)nc(-n3cccn3)n2)c1 ; CCOc1nc(N2CCNC3(COC3)C2)nc(-n2cccn2)n1 ; FC1(F)CCN(c2nc(N3CCNC4(COC4)C3)nc(-n3cccn3)n2)CC1 ; c1cnn(-c2nc(N3CCCC3)nc(N3CCNC4(COC4)C3)n2)c1]

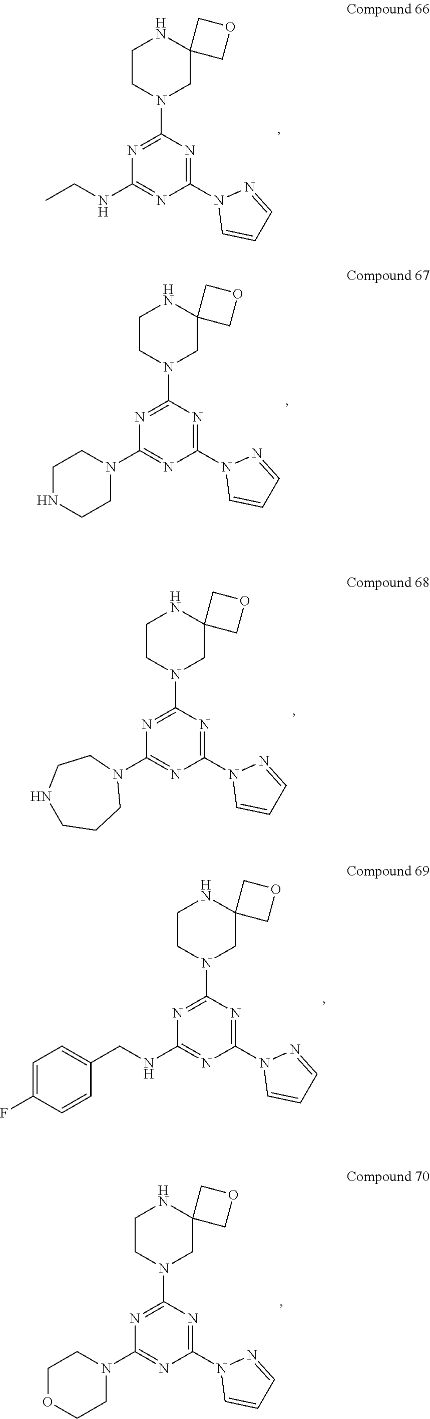 [SMILES: CCNc1nc(N2CCNC3(COC3)C2)nc(-n2cccn2)n1 ; c1cnn(-c2nc(N3CCNCC3)nc(N3CCNC4(COC4)C3)n2)c1 ; c1cnn(-c2nc(N3CCCNCC3)nc(N3CCNC4(COC4)C3)n2)c1 ; Fc1ccc(CNc2nc(N3CCNC4(COC4)C3)nc(-n3cccn3)n2)cc1 ; c1cnn(-c2nc(N3CCOCC3)nc(N3CCNC4(COC4)C3)n2)c1]

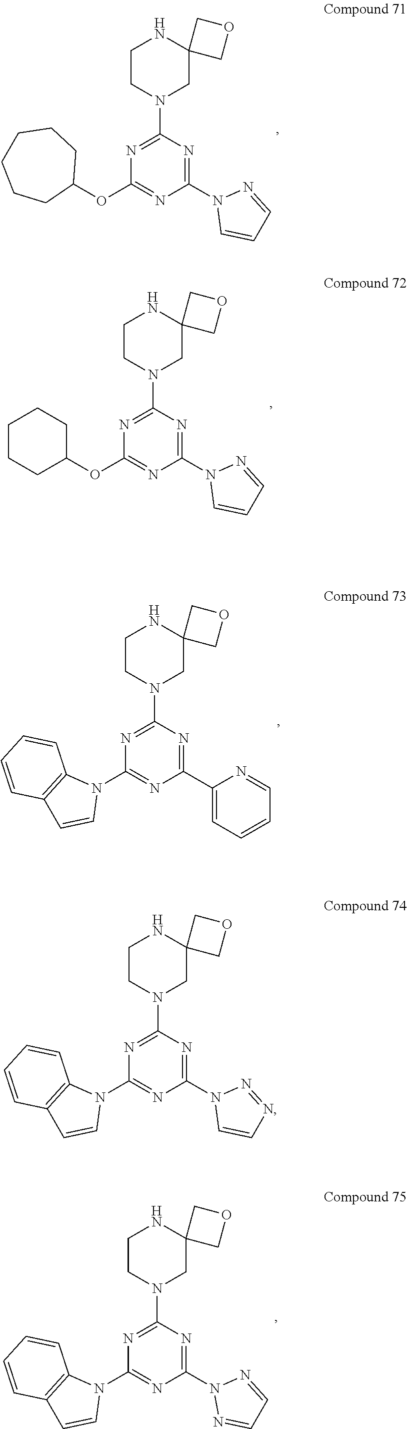 [SMILES: c1cnn(-c2nc(OC3CCCCCC3)nc(N3CCNC4(COC4)C3)n2)c1 ; c1cnn(-c2nc(OC3CCCCC3)nc(N3CCNC4(COC4)C3)n2)c1 ; c1ccc(-c2nc(N3CCNC4(COC4)C3)nc(-n3ccc4ccccc43)n2)nc1 ; c1ccc2c(c1)ccn2-c1nc(N2CCNC3(COC3)C2)nc(-n2ccnn2)n1 ; c1ccc2c(c1)ccn2-c1nc(N2CCNC3(COC3)C2)nc(-n2nccn2)n1]

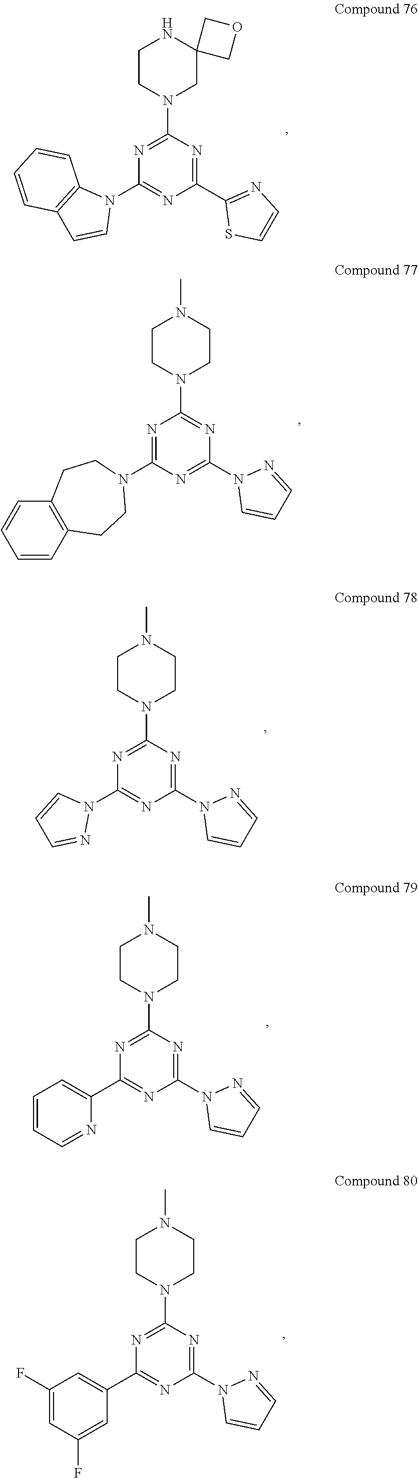 [SMILES: c1ccc2c(c1)ccn2-c1nc(-c2nccs2)nc(N2CCNC3(COC3)C2)n1 ; CN1CCN(c2nc(N3CCc4ccccc4CC3)nc(-n3cccn3)n2)CC1 ; CN1CCN(c2nc(-n3cccn3)nc(-n3cccn3)n2)CC1 ; CN1CCN(c2nc(-c3ccccn3)nc(-n3cccn3)n2)CC1 ; CN1CCN(c2nc(-c3cc(F)cc(F)c3)nc(-n3cccn3)n2)CC1]

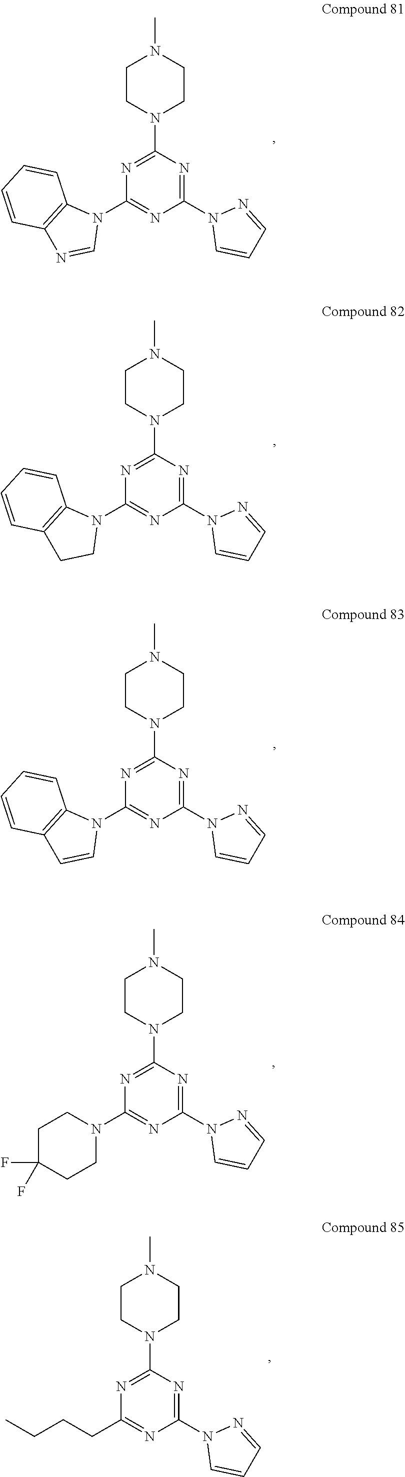 [SMILES: CN1CCN(c2nc(-n3cccn3)nc(-n3cnc4ccccc43)n2)CC1 ; CN1CCN(c2nc(N3CCc4ccccc43)nc(-n3cccn3)n2)CC1 ; CN1CCN(c2nc(-n3cccn3)nc(-n3ccc4ccccc43)n2)CC1 ; CN1CCN(c2nc(N3CCC(F)(F)CC3)nc(-n3cccn3)n2)CC1 ; CCCCc1nc(N2CCN(C)CC2)nc(-n2cccn2)n1]

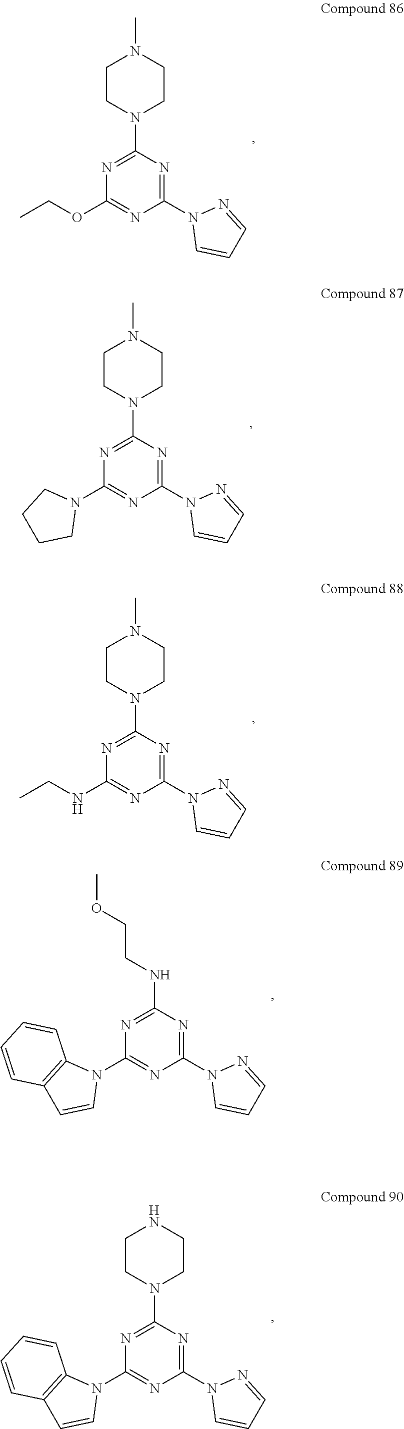 [SMILES: CCOc1nc(N2CCN(C)CC2)nc(-n2cccn2)n1 ; CN1CCN(c2nc(N3CCCC3)nc(-n3cccn3)n2)CC1 ; CCNc1nc(N2CCN(C)CC2)nc(-n2cccn2)n1 ; COCCNc1nc(-n2cccn2)nc(-n2ccc3ccccc32)n1 ; c1ccc2c(c1)ccn2-c1nc(N2CCNCC2)nc(-n2cccn2)n1]

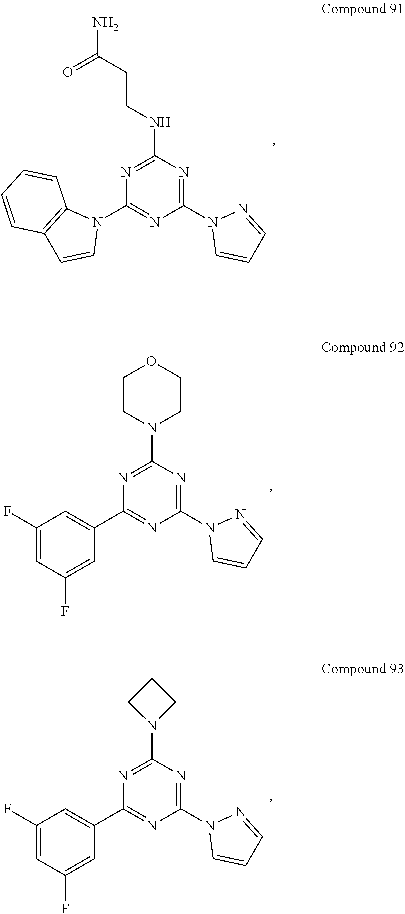 [SMILES: NC(=O)CCNc1nc(-n2cccn2)nc(-n2ccc3ccccc32)n1 ; Fc1cc(F)cc(-c2nc(N3CCOCC3)nc(-n3cccn3)n2)c1 ; Fc1cc(F)cc(-c2nc(N3CCC3)nc(-n3cccn3)n2)c1]

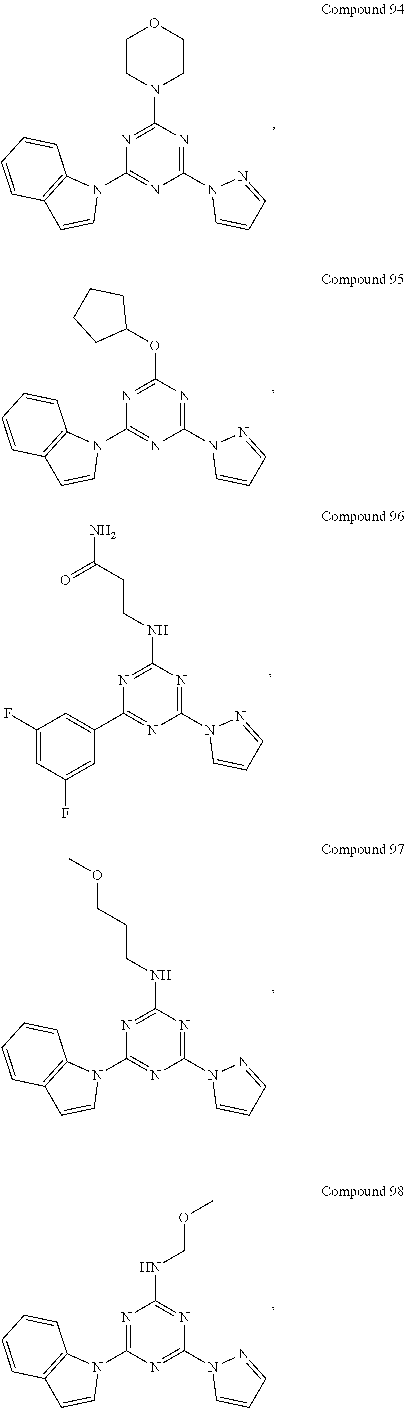

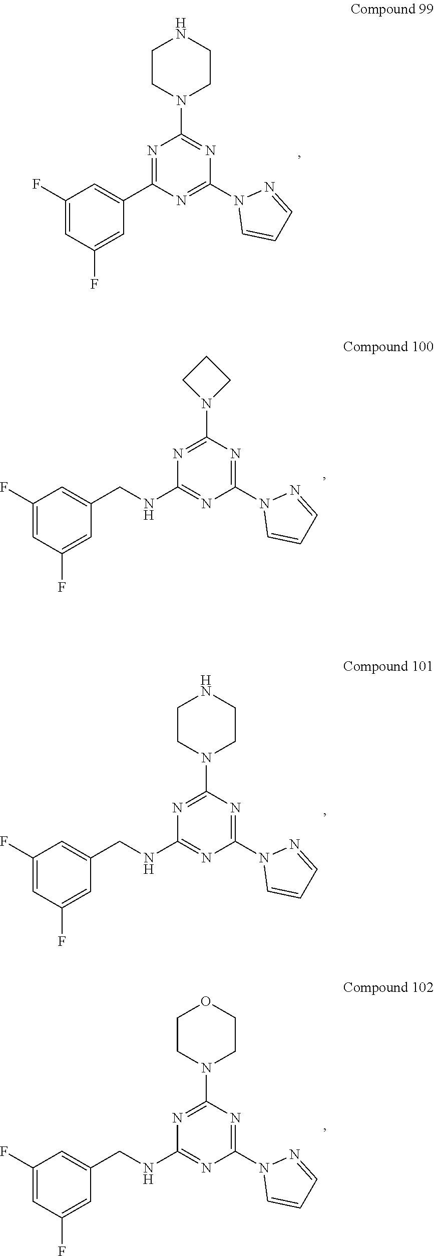 [SMILES: Fc1cc(F)cc(-c2nc(N3CCNCC3)nc(-n3cccn3)n2)c1 ; Fc1cc(F)cc(CNc2nc(N3CCC3)nc(-n3cccn3)n2)c1 ; Fc1cc(F)cc(CNc2nc(N3CCNCC3)nc(-n3cccn3)n2)c1 ; Fc1cc(F)cc(CNc2nc(N3CCOCC3)nc(-n3cccn3)n2)c1]

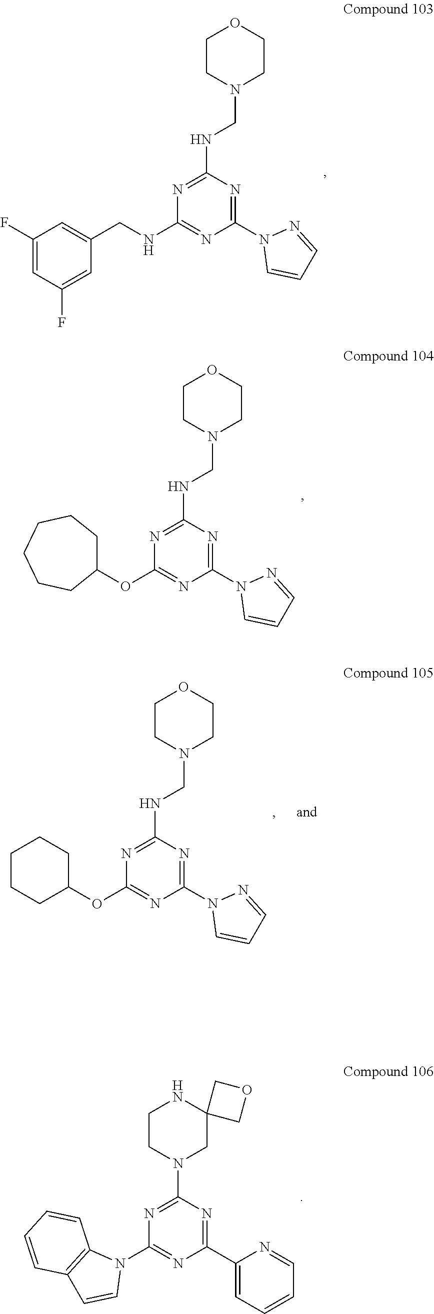 [SMILES: Fc1cc(F)cc(CNc2nc(NCN3CCOCC3)nc(-n3cccn3)n2)c1 ; c1cnn(-c2nc(NCN3CCOCC3)nc(OC3CCCCCC3)n2)c1 ; c1cnn(-c2nc(NCN3CCOCC3)nc(OC3CCCCC3)n2)c1 ; c1ccc(-c2nc(N3CCNC4(COC4)C3)nc(-n3ccc4ccccc43)n2)nc1]

Any one of the directly preceding structures may be further optionally substituted by replacing one or more —H atoms of any carbon or nitrogen atom present within the structure.

In some embodiments, the triazine compound having the structure of Formula (I) is a compound selected from one or more of the following:
ethyl 3-((4-(1H-pyrazol-1-yl)-6-(1,2,4,5-tetrahydro-3H-benzo[d]azepin-3-yl)-1,3,5-triazin-2-yl)amino)propanoate;
ethyl 3-((4,6-di(1H-pyrazol-1-yl)-1,3,5-triazin-2-yl)amino)propanoate;
ethyl 3-((4-(1H-pyrazol-1-yl)-6-(pyridin-2-yl)-1,3,5-triazin-2-yl)amino)propanoate;
ethyl 3-((4-(1H-benzo[d]imidazol-1-yl)-6-(1H-pyrazol-1-yl)-1,3,5-triazin-2-yl)amino)propanoate;
ethyl 3-((4-(indolin-1-yl)-6-(1H-pyrazol-1-yl)-1,3,5-triazin-2-yl)amino)propanoate;
ethyl 3-((4-(1H-indol-1-yl)-6-(1H-pyrazol-1-yl)-1,3,5-triazin-2-yl)amino)propanoate;
ethyl 3-((4-pentyl-6-(1H-pyrazol-1-yl)-1,3,5-triazin-2-yl)amino)propanoate;
ethyl 3-((4-(3,5-difluorophenyl)-6-(1H-pyrazol-1-yl)-1,3,5-triazin-2-yl)amino)propanoate;
ethyl 3-((4-ethoxy-6-(1H-pyrazol-1-yl)-1,3,5-triazin-2-yl)amino)propanoate;
ethyl 3-((4-(4,4-difluoropiperidin-1-yl)-6-(1H-pyrazol-1-yl)-1,3,5-triazin-2-yl)amino)propanoate;
ethyl 3-((4-(1H-pyrazol-1-yl)-6-(pyrrolidin-1-yl)-1,3,5-triazin-2-yl)amino)propanoate;
ethyl 3-((4-(ethylamino)-6-(1H-pyrazol-1-yl)-1,3,5-triazin-2-yl)amino)propanoate;
ethyl 3-((4-(piperazin-1-yl)-6-(1H-pyrazol-1-yl)-1,3,5-triazin-2-yl)amino)propanoate;
ethyl 3-((4-(1,4-diazepan-1-yl)-6-(1H-pyrazol-1-yl)-1,3,5-triazin-2-yl)amino)propanoate;
ethyl 3-((4-((4-fluorobenzyl)amino)-6-(1H-pyrazol-1-yl)-1,3,5-triazin-2-yl)amino)propanoate;
ethyl 3-((4-morpholino-6-(1H-pyrazol-1-yl)-1,3,5-triazin-2-yl)amino)propanoate;
ethyl 3-((4-(cycloheptyloxy)-6-(1H-pyrazol-1-yl)-1,3,5-triazin-2-yl)amino)propanoate;
ethyl 3-((4-(cyclohexyloxy)-6-(1H-pyrazol-1-yl)-1,3,5-triazin-2-yl)amino)propanoate;
2-((4-(4-fluorophenyl)-6-(1H-pyrazol-1-yl)-1,3,5-triazin-2-yl)amino)acetamide;
3-((4-(cycloheptyloxy)-6-(1H-pyrazol-1-yl)-1,3,5-triazin-2-yl)amino)propanamide;
N-(2-(1H-1,2,4-triazol-1-yl)ethyl)-4-(indolin-1-yl)-6-(1H-pyrazol-1-yl)-1,3,5-triazin-2-amine;
4-(cycloheptyloxy)-N-(morpholinomethyl)-6-(pyridin-2-yl)-1,3,5-triazin-2-amine;
(S)—N-(1-(4-(3,5-difluorophenyl)-6-(1H-pyrazol-1-yl)-1,3,5-triazin-2-yl)ethyl)-N-methyltetrahydro-2H-pyran-4-amine;
N-(2-(1H-tetrazol-1-yl)ethyl)-4-(1H-indol-1-yl)-6-(1H-pyrazol-1-yl)-1,3,5-triazin-2-amine;
4-(1H-benzo[d]imidazol-1-yl)-N-(3-ethoxypropyl)-6-(1H-pyrazol-1-yl)-1,3,5-triazin-2-amine;
3-((4-(3,5-difluorophenyl)-6-(1H-pyrazol-1-yl)-1,3,5-triazin-2-yl)amino)propan-1-ol;
4-ethoxy-N-(2-methoxyethyl)-6-(1H-pyrazol-1-yl)-1,3,5-triazin-2-amine;
4-(4,4-difluoropiperidin-1-yl)-6-(1H-pyrazol-1-yl)-N-(tetrahydro-2H-pyran-4-yl)-1,3,5-triazin-2-amine;
1-(((4-(1H-pyrazol-1-yl)-6-(pyrrolidin-1-yl)-1,3,5-triazin-2-yl)amino)methyl)urea;
1-ethyl-3-(((4-(ethylamino)-6-(1H-pyrazol-1-yl)-1,3,5-triazin-2-yl)amino)methyl)urea;
4-((4-(4-methylpiperazin-1-yl)-6-(1H-pyrazol-1-yl)-1,3,5-triazin-2-yl)amino)butan-2-one;
3-((4-(1,4-diazepan-1-yl)-6-(1H-pyrazol-1-yl)-1,3,5-triazin-2-yl)amino)-1-(pyrrolidin-1-yl)propan-1-one;
1-(aziridin-1-yl)-3-((4-((4-fluorobenzyl)amino)-6-(1H-pyrazol-1-yl)-1,3,5-triazin-2-yl)amino)propan-1-one;
3-((4-morpholino-6-(1H-pyrazol-1-yl)-1,3,5-triazin-2-yl)amino)-1-(1H-pyrrol-1-yl)propan-1-one;
3-((4-(cycloheptyloxy)-6-(1H-pyrazol-1-yl)-1,3,5-triazin-2-yl)amino)-1-(1H-pyrrol-2-yl)propan-1-one;
1-(oxazol-2-yl)-3-((4-pentyl-6-(1H-pyrazol-1-yl)-1,3,5-triazin-2-yl)amino)propan-1-one;
4-(cyclohexyloxy)-N-isopropyl-6-(1H-pyrazol-1-yl)-1,3,5-triazin-2-amine;
4-(cyclohexyloxy)-N-isobutyl-6-(1H-pyrazol-1-yl)-1,3,5-triazin-2-amine;
1-((4-(cyclohexyloxy)-6-(1H-pyrazol-1-yl)-1,3,5-triazin-2-yl)amino)ethan-1-ol;
N-butyl-4-(cyclohexyloxy)-6-(1H-pyrazol-1-yl)-1,3,5-triazin-2-amine;
4-(cyclohexyloxy)-N-cyclopropyl-6-(1H-pyrazol-1-yl)-1,3,5-triazin-2-amine;
N-cyclobutyl-4-(cyclohexyloxy)-6-(1H-pyrazol-1-yl)-1,3,5-triazin-2-amine;
4-(cyclohexylmethyl)-6-(1H-pyrazol-1-yl)-N-(tetrahydro-2H-pyran-4-yl)-1,3,5-triazin-2-amine;
4-(cyclohexylmethyl)-6-(1H-pyrazol-1-yl)-N-(tetrahydrofuran-3-yl)-1,3,5-triazin-2-amine;
4-(cyclohexyloxy)-N-(piperidin-4-yl)-6-(1H-pyrazol-1-yl)-1,3,5-triazin-2-amine;
4-(cyclohexylmethyl)-6-(1H-pyrazol-1-yl)-N-(pyridin-2-yl)-1,3,5-triazin-2-amine;
ethyl (4-(cyclohexyloxy)-6-(1H-pyrazol-1-yl)-1,3,5-triazin-2-yl)glycinate;
2-((4-(cyclohexyloxy)-6-(1H-pyrazol-1-yl)-1,3,5-triazin-2-yl)amino)acetamide;
4-(cyclohexyloxy)-N-(morpholinomethyl)-6-(1H-pyrazol-1-yl)-1,3,5-triazin-2-amine;
4-(cyclohexyloxy)-N-(piperidin-4-ylmethyl)-6-(1H-pyrazol-1-yl)-1,3,5-triazin-2-amine;
4-(cyclohexyloxy)-6-(1H-pyrazol-1-yl)-N-(1H-pyrrol-2-yl)-1,3,5-triazin-2-amine;
4-(cyclohexyloxy)-6-(1H-pyrazol-1-yl)-N-(trifluoromethyl)-1,3,5-triazin-2-amine;
4-(cyclohexyloxy)-N-(2,3-difluorophenyl)-6-(1H-pyrazol-1-yl)-1,3,5-triazin-2-amine;
4-(cyclohexyloxy)-6-(1H-pyrazol-1-yl)-N-(thiophen-2-yl)-1,3,5-triazin-2-amine;
8-(4-(1H-pyrazol-1-yl)-6-(1,2,4,5-tetrahydro-3H-benzo[d]azepin-3-yl)-1,3,5-triazin-2-yl)-2-oxa-5,8-diazaspiro[3.5]nonane;
8-(4,6-di(1H-pyrazol-1-yl)-1,3,5-triazin-2-yl)-2-oxa-5,8-diazaspiro[3.5]nonane;
8-(4-(1H-pyrazol-1-yl)-6-(pyridin-2-yl)-1,3,5-triazin-2-yl)-2-oxa-5,8-diazaspiro[3.5]nonane;
8-(4-(1H-benzo[d]imidazol-1-yl)-6-(1H-pyrazol-1-yl)-1,3,5-triazin-2-yl)-2-oxa-5,8-diazaspiro[3.5]nonane;
8-(4-(indolin-1-yl)-6-(1H-pyrazol-1-yl)-1,3,5-triazin-2-yl)-2-oxa-5,8-diazaspiro[3.5]nonane;
8-(4-(1H-indol-1-yl)-6-(1H-pyrazol-1-yl)-1,3,5-triazin-2-yl)-2-oxa-5,8-diazaspiro[3.5]nonane;
8-(4-pentyl-6-(1H-pyrazol-1-yl)-1,3,5-triazin-2-yl)-2-oxa-5,8-diazaspiro[3.5]nonane;
8-(4-(3,5-difluorophenyl)-6-(1H-pyrazol-1-yl)-1,3,5-triazin-2-yl)-2-oxa-5,8-diazaspiro[3.5]nonane;
8-(4-ethoxy-6-(1H-pyrazol-1-yl)-1,3,5-triazin-2-yl)-2-oxa-5,8-diazaspiro[3.5]nonane;
8-(4-(4,4-difluoropiperidin-1-yl)-6-(1H-pyrazol-1-yl)-1,3,5-triazin-2-yl)-2-oxa-5,8-diazaspiro[3.5]nonane;
8-(4-(1H-pyrazol-1-yl)-6-(pyrrolidin-1-yl)-1,3,5-triazin-2-yl)-2-oxa-5,8-diazaspiro[3.5]nonane;
N-ethyl-4-(1H-pyrazol-1-yl)-6-(2-oxa-5,8-diazaspiro[3.5]nonan-8-yl)-1,3,5-triazin-2-amine;
8-(4-(piperazin-1-yl)-6-(1H-pyrazol-1-yl)-1,3,5-triazin-2-yl)-2-oxa-5,8-diazaspiro[3.5]nonane;
8-(4-(1,4-diazepan-1-yl)-6-(1H-pyrazol-1-yl)-1,3,5-triazin-2-yl)-2-oxa-5,8-diazaspiro[3.5]nonane;
N-(4-fluorobenzyl)-4-(1H-pyrazol-1-yl)-6-(2-oxa-5,8-diazaspiro[3.5]nonan-8-yl)-1,3,5-triazin-2-amine;
8-(4-morpholino-6-(1H-pyrazol-1-yl)-1,3,5-triazin-2-yl)-2-oxa-5,8-diazaspiro[3.5]nonane;
8-(4-(cycloheptyloxy)-6-(1H-pyrazol-1-yl)-1,3,5-triazin-2-yl)-2-oxa-5,8-diazaspiro[3.5]nonane;
8-(4-(cyclohexyloxy)-6-(1H-pyrazol-1-yl)-1,3,5-triazin-2-yl)-2-oxa-5,8-diazaspiro[3.5]nonane;
8-(4-(1H-indol-1-yl)-6-(pyridin-2-yl)-1,3,5-triazin-2-yl)-2-oxa-5,8-diazaspiro[3.5]nonane;
8-(4-(1H-indol-1-yl)-6-(1H-1,2,3-triazol-1-yl)-1,3,5-triazin-2-yl)-2-oxa-5,8-diazaspiro[3.5]nonane;
8-(4-(1H-indol-1-yl)-6-(2H-1,2,3-triazol-2-yl)-1,3,5-triazin-2-yl)-2-oxa-5,8-diazaspiro[3.5]nonane;
8-(4-(1H-indol-1-yl)-6-(thiazol-2-yl)-1,3,5-triazin-2-yl)-2-oxa-5,8-diazaspiro[3.5]nonane;
2-(4-methylpiperazin-1-yl)-4-(1H-pyrazol-1-yl)-6-(pyridin-2-yl)-1,3,5-triazine;
2-(4-methylpiperazin-1-yl)-4,6-di(1H-pyrazol-1-yl)-1,3,5-triazine;
3-(4-(4-methylpiperazin-1-yl)-6-(1H-pyrazol-1-yl)-1,3,5-triazin-2-yl)-2,3,4,5-tetrahydro-1H-benzo[d]azepine;
2-(3,5-difluorophenyl)-4-(4-methylpiperazin-1-yl)-6-(1H-pyrazol-1-yl)-1,3,5-triazine;
1-(4-(4-methylpiperazin-1-yl)-6-(1H-pyrazol-1-yl)-1,3,5-triazin-2-yl)-1H-benzo[d]imidazole;
1-(4-(4-methylpiperazin-1-yl)-6-(1H-pyrazol-1-yl)-1,3,5-triazin-2-yl)indoline;
1-(4-(4-methylpiperazin-1-yl)-6-(1H-pyrazol-1-yl)-1,3,5-triazin-2-yl)-1H-indole;
2-(4,4-difluoropiperidin-1-yl)-4-(4-methylpiperazin-1-yl)-6-(1H-pyrazol-1-yl)-1,3,5-triazine;
2-butyl-4-(4-methylpiperazin-1-yl)-6-(1H-pyrazol-1-yl)-1,3,5-triazine;
2-ethoxy-4-(4-methylpiperazin-1-yl)-6-(1H-pyrazol-1-yl)-1,3,5-triazine;
2-(4-methylpiperazin-1-yl)-4-(1H-pyrazol-1-yl)-6-(pyrrolidin-1-yl)-1,3,5-triazine;
N-ethyl-4-(4-methylpiperazin-1-yl)-6-(1H-pyrazol-1-yl)-1,3,5-triazin-2-amine;
4-(1H-indol-1-yl)-N-(2-methoxyethyl)-6-(1H-pyrazol-1-yl)-1,3,5-triazin-2-amine;
1-(4-(piperazin-1-yl)-6-(1H-pyrazol-1-yl)-1,3,5-triazin-2-yl)-1H-indole;
3-((4-(1H-indol-1-yl)-6-(1H-pyrazol-1-yl)-1,3,5-triazin-2-yl)amino)propanamide;
4-(4-(3,5-difluorophenyl)-6-(1H-pyrazol-1-yl)-1,3,5-triazin-2-yl)morpholine;
2-(azetidin-1-yl)-4-(3,5-difluorophenyl)-6-(1H-pyrazol-1-yl)-1,3,5-triazine;
4-(4-(1H-indol-1-yl)-6-(1H-pyrazol-1-yl)-1,3,5-triazin-2-yl)morpholine;
1-(4-(cyclopentyloxy)-6-(1H-pyrazol-1-yl)-1,3,5-triazin-2-yl)-1H-indole;
3-((4-(3,5-difluorophenyl)-6-(1H-pyrazol-1-yl)-1,3,5-triazin-2-yl)amino)propanamide;
4-(1H-indol-1-yl)-N-(3-methoxypropyl)-6-(1H-pyrazol-1-yl)-1,3,5-triazin-2-amine;
4-(1H-indol-1-yl)-N-(methoxymethyl)-6-(1H-pyrazol-1-yl)-1,3,5-triazin-2-amine;
2-(3,5-difluorophenyl)-4-(piperazin-1-yl)-6-(1H-pyrazol-1-yl)-1,3,5-triazine;
4-(azetidin-1-yl)-N-(3,5-difluorobenzyl)-6-(1H-pyrazol-1-yl)-1,3,5-triazin-2-amine;
N-(3,5-difluorobenzyl)-4-(piperazin-1-yl)-6-(1H-pyrazol-1-yl)-1,3,5-triazin-2-amine;
N-(3,5-difluorobenzyl)-4-morpholino-6-(1H-pyrazol-1-yl)-1,3,5-triazin-2-amine;
N²-(3,5-difluorobenzyl)-N⁴-(morpholinomethyl)-6-(1H-pyrazol-1-yl)-1,3,5-triazine-2,4-diamine;
4-(cycloheptyloxy)-N-(morpholinomethyl)-6-(1H-pyrazol-1-yl)-1,3,5-triazin-2-amine;
4-(cyclohexyloxy)-N-(morpholinomethyl)-6-(1H-pyrazol-1-yl)-1,3,5-triazin-2-amine; and
8-(4-(1H-indol-1-yl)-6-(pyridin-2-yl)-1,3,5-triazin-2-yl)-2-oxa-5,8-diazaspiro[3.5]nonane.

In several embodiments, as disclosed elsewhere herein, the variables defined for one structural formula may be also be used for any other formula having that same variable. For example, when a variable has the same alphanumeric designation (e.g., A₂) for one formula (e.g., Formula (Id) or (I), etc.), that definition of the variable can be used in other formulae (e.g., Formula (Ic), (Ie), etc.), even where the variable is not specifically defined for those other formulae.

### Formula (Ia) Genus and Compounds

Several exemplary embodiments of structures of Formula (I) may be represented by the structure of Formula (Ia), an amino triazine, where L is —NR¹— with R¹ being —H, Aₐ is C₁-C₃ alkylene, A_{b} is —C(═O)—, A_{c} is not present (e.g., is a single bond), n is an integer equal to 1, 2, or 3, and each other variable is as defined elsewhere herein:

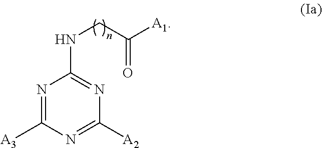

Formula (Ia) may be represented by any one of Compounds 1, 2, 3, 4, 5, 6, 7, 8, 9, 10, 11, 12, 13, 14, 15, 16, 17, 18, 26, 36, 91, 96, or others. Formula (Ib) Genus and Compounds

In several embodiments, the compound of Formula (I) (or Formula (Ia)) is further represented by the structure of Formula (Ib), where L is —NR¹— with R¹ being —H, Aₐ is C₁-C₃ alkylene, A_{b} is —C(═O)—, A_{c} is a single bond, A₂ is pyrazole, and each other variable is as defined elsewhere herein:

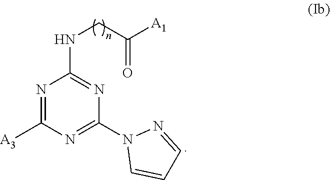

Several embodiments of the structure of Formula (I) (or Formula (Ib)), are represented by Formula (Ib-i), where L is —NR¹— with R¹ being —H; Aₐ is ethylene; A_{b} is —C(═O)—; A_{c} is a single bond; A₁ is ethoxy, and each other variable is as defined elsewhere herein:

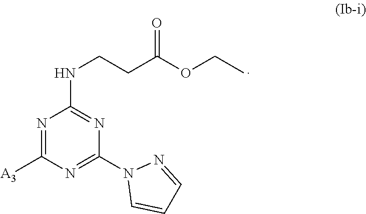

In several embodiments of Formula (Ib-i), where A₃ is selected from:

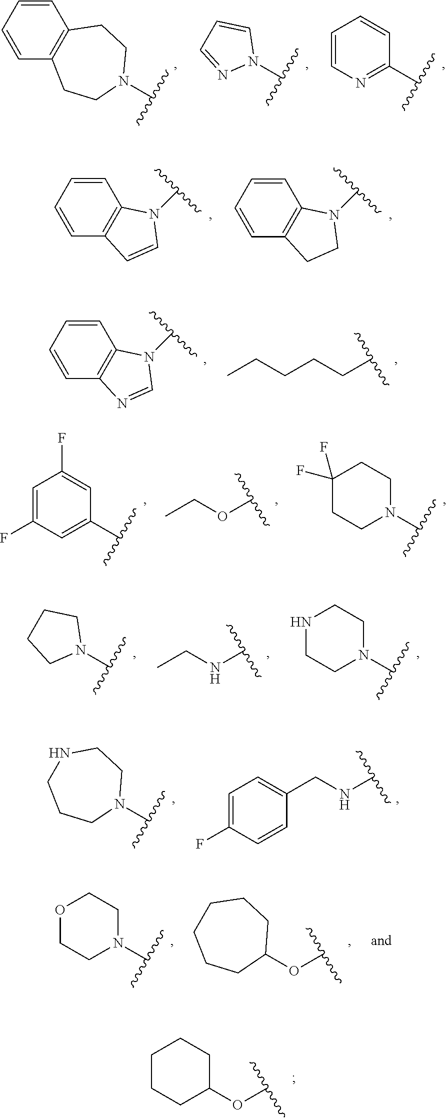

and Formula (Ib-i) may be represented by any one of the Compounds 1, 2, 3, 4, 5, 6, 7, 8, 9, 10, 11, 12, 13, 14, 15, 16, 17, 18, or others.

Several embodiments of the structure of Formula (I), are represented by Formula (Ib-ii), where L is —NR¹— with R¹ being —H; Aₐ is ethylene; A_{b} is —C(═O)—; and A_{c} is a single bond (where the other variables are as disclosed anywhere else herein):

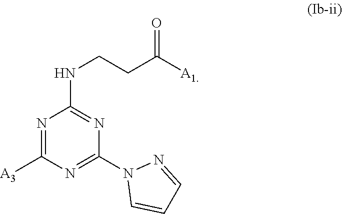

In several embodiments of Formula (Ib-ii), where A₁ is selected from:

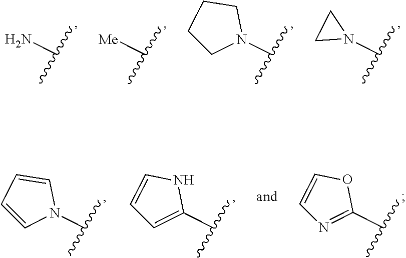

and A₃ is selected from:

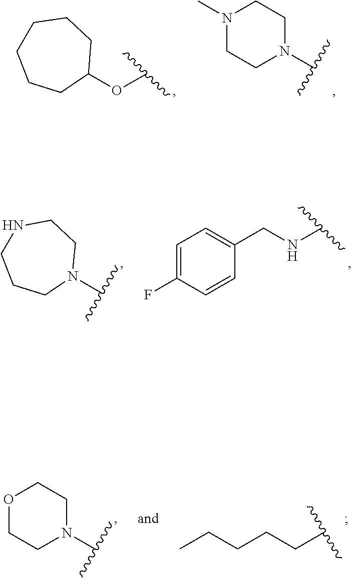

and each other variable is as defined elsewhere herein, Formula (Ib-ii) may be represented by any one of Compounds 20, 31, 32, 33, 34, 35, 36, or others.

### Formula (Ic) Genus and Compounds

In several embodiments, where A₂ is a 5 or 6 membered heteroaromatic ring or a 5 or 6 membered heterocyclyl ring represented by the symbol:

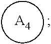

Formula (I) is further represented by the structure of Formula (Ic) where other variable is as defined elsewhere herein:

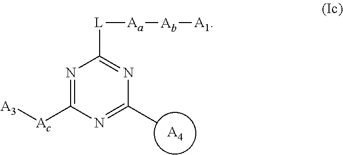

Formula (Ic) may be represented by any one of Compounds 41, 42, 45, 51, 53, 54, 89, 97, or others.

Several embodiments of the structure of Formula (I) (or Formula (Ic)), are further represented by Formula (Ic-i), where A_{c} is —O—, A₂ is pyrazole, A₃ is cyclohexane, and each other variable is as defined elsewhere herein:

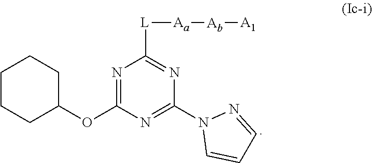

In several embodiments of Formula (Ic-i), where L is —NR¹— with R¹ being —H, Aₐ and A_{b} are not present, A₁ is selected from:

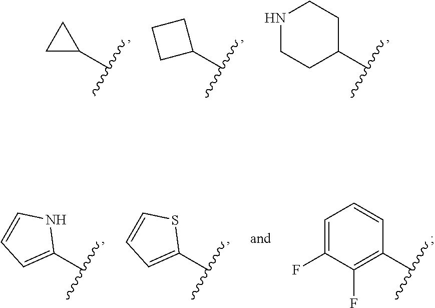

and each other variable is as defined elsewhere herein, Formula (Ic-i) may be represented by any one of Compounds 41, 42, 45, 51, 53, 54, or others.

In several embodiments of Formula (Ic-i), where L is —NR¹— with R¹ being —H, Aₐ and A_{b} are not present, and A₁ is selected from (CH₃)₂CH—, (CH₃)₂CHCH₂—, CH₃(CH₂)₃—, CH₃(HO)CH—, and CF₃, and each other variable is as defined elsewhere herein, Formula (Ic-i) may be represented by any one of Compounds 37, 38, 39, 40, 52 or others.

In several embodiments of Formula (Ic-i), where L is —NR¹— with R¹ being —H, Aₐ is methylene, A_{b} is not present, A₁ is selected from:

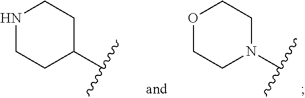 [SMILES: *C1CCNCC1 ; *N1CCOCC1]

and each other variable is as defined elsewhere herein, Formula (Ic-i) may be represented by any one of Compounds 49, 50, or others.

In several embodiments of Formula (Ic-i), where L is —NR¹— with R¹ being —H, Aₐ is methylene, A_{b} is —C(═O)—, A₁ is selected from CH₃CH₂O— and NH₂—, and each other variable is as defined elsewhere herein, Formula (Ici) may be represented by any one Compounds 47, 48, or others.

Several embodiments of the structure of Formula (I), are represented by Formula (Ic-ii), where A_{c} is —CH₂—, A₂ is pyrazole, A₃ is cyclohexane, and each other variable is as defined elsewhere herein:

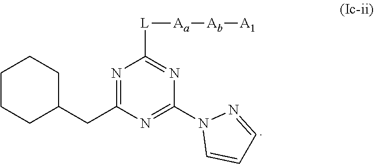

In several embodiments of Formula (Ic-ii), where L is —NR¹— with R¹ being —H, Aₐ and A_{b} are not present, A₁ is selected from:

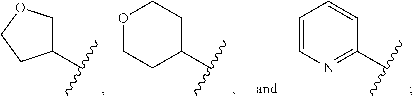

and each other variable is as defined elsewhere herein, Formula (Ic-ii) may be represented by any one of Compounds 43, 44, 46, or others.

Several embodiments of the structure of Formula (I), are represented by Formula (Ic-iii), where L is —NR¹— with R¹ being —H, Aₐ is ethylene, A_{b} and A_{c} are not present, A₂ is pyrazole, and each other variable is as defined elsewhere herein:

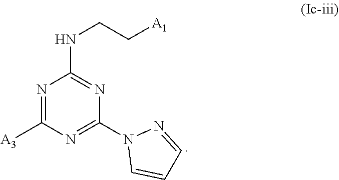

In several embodiments of Formula (Ic-iii), where A₁ is selected from:

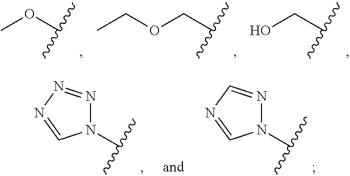

and A₃ is selected from:

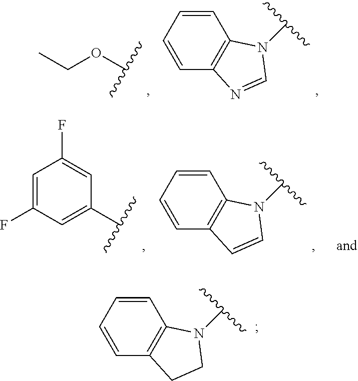

and each other variable is as defined elsewhere herein, Formula (Ic-iii) may be represented by any one of Compounds 21, 24, 25, 26, 27, or others.

Several embodiments of the structure of Formula (I), are represented by Formula (Ic-iv), where L is —NR¹— with R being —H, Aₐ is methylene, A_{b} and A_{c} are not present, A₂ is pyrazole, and each other variable is as defined elsewhere herein:

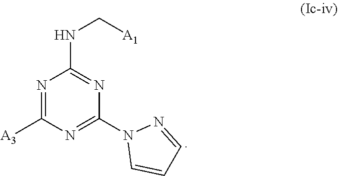

In several embodiments of Formula (Ic-iv), where A₁ is selected from: NH₂C(═O)CH₂—, NH₂C(═O)NH—, CH₃CH₂NHC(═O)NH—, and CH₃O—; and A₃ is selected from:

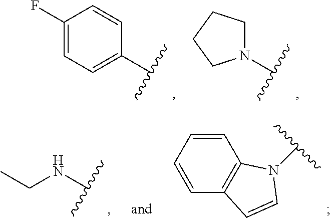

and each other variable is as defined elsewhere herein, Formula (Ic-iv) may be represented by any one of Compounds 19, 29, 30, 98, or others.

Several embodiments of the structure of Formula (I), are represented by Formula (Ic-v), where Aₐ and A_{c} are not present, A₁ is tetrahydropyran, A₂ is pyrazole, and each other variable is as defined elsewhere herein:

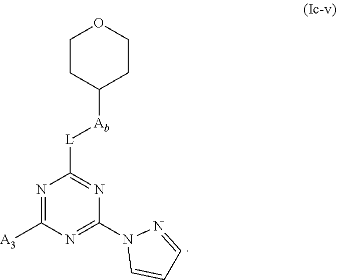

In several embodiments of Formula (Ic-v), where L is —NR¹— with R¹ being —H; A_{b} is not present; and A₃ is:

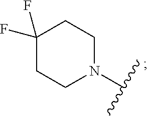 [SMILES: *N1CCC(F)(F)CC1]

and each other variable is as defined elsewhere herein, Formula (Ic-v) may be represented by Compound 28 or others.

In several embodiments of Formula (Ic-v), where L is —CR¹— with R¹ being CH₃; A_{b} is —N(CH₃)—; and A₃ is:

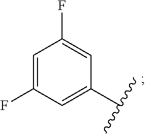 [SMILES: *c1cc(F)cc(F)c1]

and each other variable is as defined elsewhere herein, Formula (Ic-v) may be represented by Compound 23 or others.

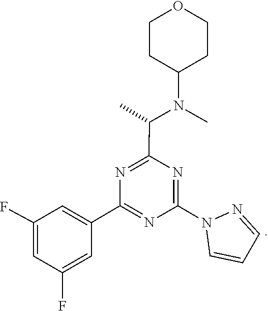 [SMILES: C[C@@H](c1nc(-c2cc(F)cc(F)c2)nc(-n2cccn2)n1)N(C)C1CCOCC1]

Several embodiments of the structure of Formula (I), are represented by Formula (Ic-vi), where L is —O—; Aₐ, A_{b}, and A_{c} are not present; and A₂ is pyrazole:

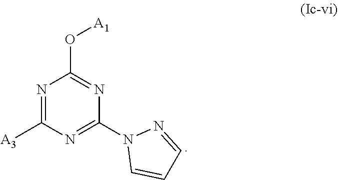

In several embodiments of Formula (Ic-vi), A₁ is:

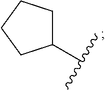 [SMILES: *C1CCCC1]

### and A₃ is:

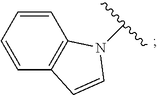 [SMILES: *n1ccc2ccccc21]

and each other variable is as defined elsewhere herein, Formula (Ic-vi) may be represented by the Compound 95 or others.

Several embodiments of the structure of Formula (I), are represented by Formula (Ic-vii), a pyridinyl triazine, where L is —NR¹— with R¹ being —H; A_{b} and A_{c} are not present; and A₂ is pyridine:

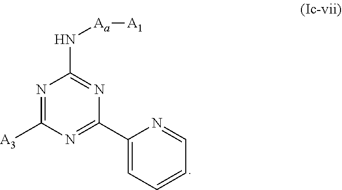

In several embodiments of Formula (Ic-vii), where Aₐ is methylene; A₁ is:

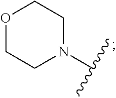 [SMILES: *N1CCOCC1]

### and A₃ is:

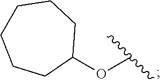 [SMILES: *OC1CCCCCC1]

and each other variable is as defined elsewhere herein, Formula (Ic-vii) may be represented by Compound 22 or others.

### Formula (Id) Genus and Compounds

In several embodiments, the compound of Formula (I) is further represented by the structure of Formula (Id), where Aₐ and A_{b} are absent, L is a heterocyclyl (e.g., L is N(R¹)₂ taken together form an optionally substituted 3-12 membered heterocycle) and A₁ is as defined elsewhere herein. In several embodiments, each instance of R¹ is C₁₋₆ alkyl. In several embodiments, one instance of R¹ is C₁₋₆ alkyl one instance of R¹ is C₁-C₆ alkamino, C₁₋₆ alkoxy, or C₁-C₆ alkylthio. In several embodiments, L forms a ring with X. In other words, L is tethered to X by two independent linkages comprising alkylene groups, where the number of alkylene groups in a linkage is an integer represented by m and o.

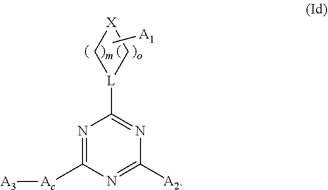

In several embodiments, X is selected from the group consisting of —CH₂—, —NH—, N (where A₁ is a substituent of X), —O—, and —S—; m is an integer equal to 1, 2, 3, or 4; and o is an integer equal to 1, 2, 3, or 4, and A₁ is a heterocyclyl group. In this instance, Formula (Id) may be represented by any one of the Compounds 55, 56, 57, 58, 59, 60, 61, 62, 63, 64, 65, 66, 67, 68, 69, 70, 71, 72, 73, 74, 75, 76, 101, or others.

Alternatively, in several embodiments, X is N, m is an integer equal to 2, o is an integer equal to 2, and A₁ is an alkyl group substituted on X. In this instance (or others), Formula (Id) may be represented by any one of the Compounds 77, 78, 79, 80, 81, 82, 83, 84, 85, 86, 87, 88, or others.

In several embodiments, the compound of Formula (Id-i) comprises a compound where Aₐ, A_{b}, and A₁ are absent, L is optionally substituted N(R′)₂ with each instance of R¹ being C₁₋₆ alkyl and L forms a ring with X, and each other variable is as disclosed elsewhere herein. In several embodiments, L (e.g., X or a methylene of L) is optionally substituted with C₁-C₆ alkyl. In this instance (or others), Formula (Id-i) may be represented by any one of the Compounds 77, 78, 79, 80, 81, 82, 83, 84, 85, 86, 87, 88, or others.

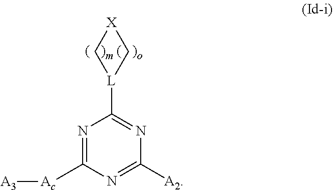

In several embodiments for Formula (Id-i), X is selected from the group consisting of —CH₂—, —NH—, and —O—; m is an integer equal to 1, 2, 3, or 4; and o is an integer equal to 1, 2, 3, or 4. In this instance (or others), Formula (Id-1) may be represented by any one of the Compounds 92, 93, 94, 99, or others.

Several embodiments of the structure of Formula (I), are represented by Formula (Id-i), where Aₐ, A_{b}, and A₁ are absent; A₂ is pyrazole; and A₃ is 3,5-difluorophenyl:

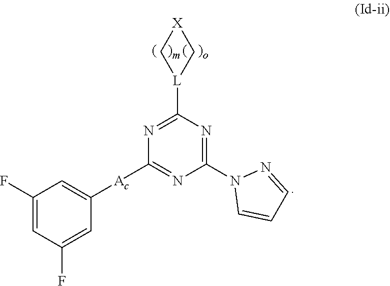

In several embodiments of Formula (Id-ii), where A_{c} is absent; X is selected from —NH— or —O—; m is 2; o is 2; and each other variable is as defined elsewhere herein, Formula (Id-i) may be represented by Compounds 92, 99, (or others).

In several embodiments of Formula (Id-i), where A_{c} is absent; X is selected from —CH₂—; m is 1; o is 1; and each other variable is as defined elsewhere herein, Formula (Id-i) may be represented by Compound 93 or others.

In several embodiments of Formula (Id-ii), where A_{c} is —CH₂NH—; X is selected from —NH— or —O—; m is 2; o is 2; and each other variable is as defined elsewhere herein, Formula (Id-i) may be represented by Compounds 101, 102, or others.

In several embodiments of Formula (Id-ii), where A_{c} is —CH₂NH—; X is selected from —CH₂—; m is 1; o is 1; and each other variable is as defined elsewhere herein, Formula (Id-i) may be represented by Compound 100 or others.

### Formula (Ie) Genus and Compounds

In several embodiments, the compound of Formula (I) is further represented by the structure of Formula (Ie), where L is —NR¹— with R¹ being —H; Aₐ is methylene, A_{b} and A_{c} are absent; A₁ is morpholine; and A₂ is pyrazole:

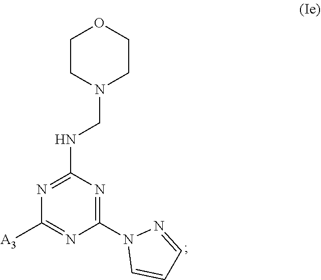

In several embodiments of Formula (Ie), where A₃ is selected from:

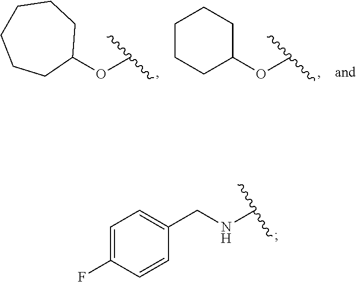

and each other variable is as defined elsewhere herein, Formula (Ie) may be represented by Compound 103, 104, 105, or others.

### Methods of Making Compounds of Formula I

The compounds disclosed herein may be synthesized by methods described below, or by modification of these methods. Ways of modifying the methodology include, among others, temperature, solvent, reagents etc., known to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and P. G. M. Green, T. W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

In the following schemes, protecting groups are selected for their compatibility with the requisite synthetic steps as well as compatibility of the introduction and deprotection steps with the overall synthetic schemes (P. G. M. Green, T. W. Wutts, Protecting Groups in Organic Synthesis (3rd ed.) Wiley, New York (1999)).

If the compounds of the present technology contain one or more chiral centers, such compounds can be prepared or isolated as pure stereoisomers, i.e., as individual enantiomers or d(1) stereoisomers, or as stereoisomer-enriched mixtures. All such stereoisomers (and enriched mixtures) are included within the scope of the present technology, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Emka-Chemce or Sigma (St. Louis, Missouri, USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

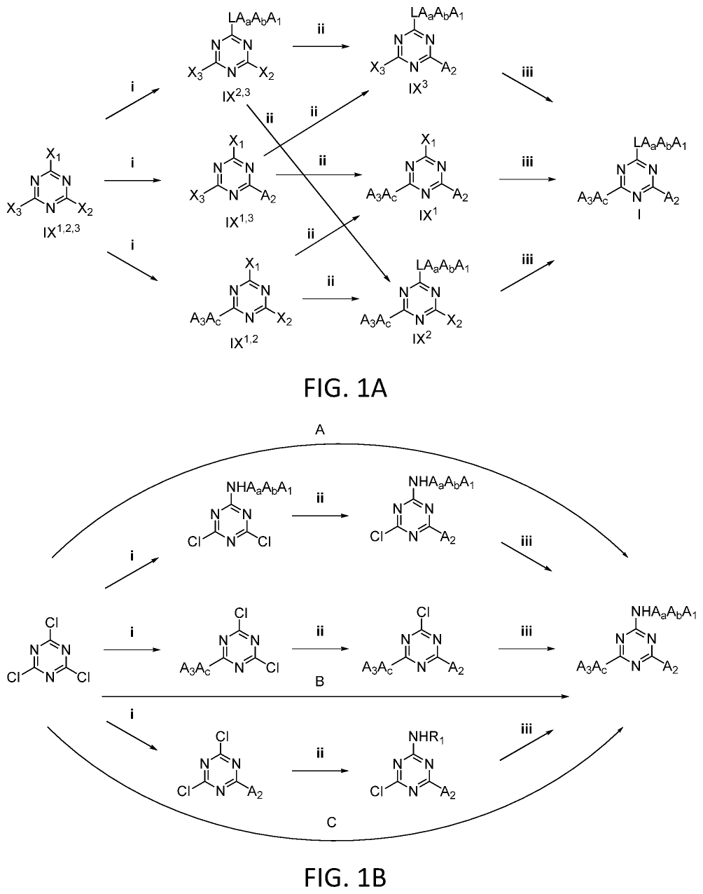

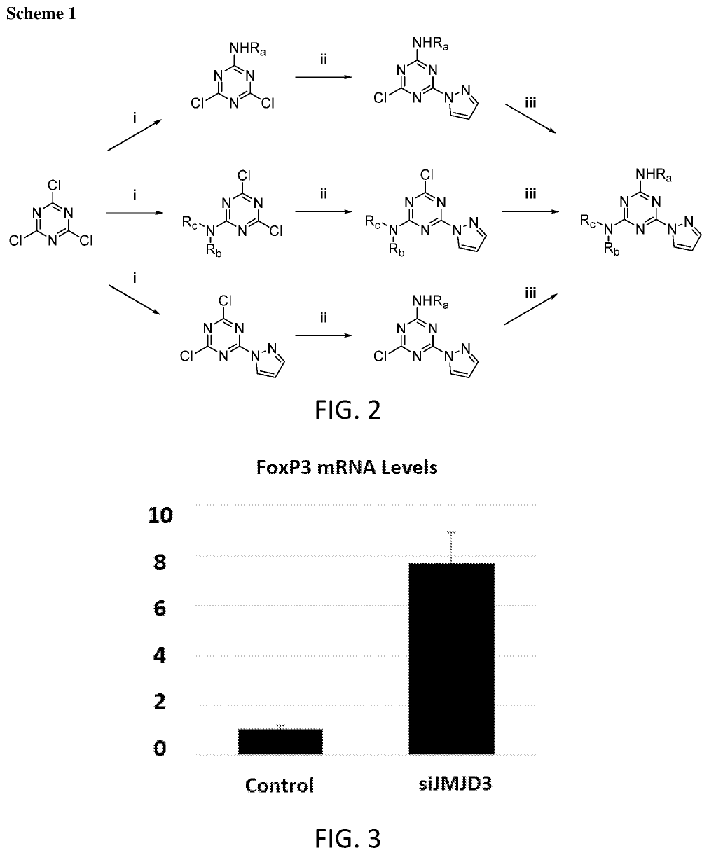

Several exemplary embodiments pertain to methods of synthesizing compounds of Formula (I) and/or intermediates of compounds of Formula (I) (as shown in FIGS. 1A-2). FIG. 1A provides a number of possible routes to compounds of Formula (I). In several embodiments, the variables of FIG. 1A are as disclosed elsewhere herein and X₁, X₂, and X₃, are each a halogen (e.g., —Cl). In several embodiments, a compound of Formula (I) may be prepared, as shown in FIG. 1A or 1B. In several embodiments, referring to either FIG. 1A or 1B, the method may comprise any one or more of steps (i), (ii), (iii), or combinations of any of the foregoing. In several embodiments, any one of steps (i), (ii), (iii), can be omitted. For example, in several embodiments, the method may include step i, but not ii or iii. In several embodiments, the method may include step i and ii, but not iii. In several embodiments, the method may include steps i, ii, and iii. In several embodiments, the method may include step ii and iii, but not step i. The method may include step ii, but not steps i or iii. The method may include step iii, but not step i or ii.

In several embodiments, step (i) includes providing a reagent that displaces one of X₁, X₂, or X₃ (e.g., H-LAₐA_{b}A₁, H-A₂, or H-A_{c}A₃ (e.g., 1.0 eq.)). In several embodiments, step (i) is carried out in the presence of base (e.g., DIPEA, 1.5 eq.). In several embodiments, step (i) is carried out in the presence of a polar aprotic solvent (e.g., THF). In several embodiments, step (i) is carried out at room temperature or lower (e.g., −78° C. to R.T.).

In several embodiments, step (ii) includes providing a reagent (e.g., H-LAₐA_{b}A₁, A₂, or A_{c}A₃ (e.g., 1.0 eq.)) that displaces one of the remaining X groups (e.g., X₁, X₂, or X₃). In several embodiments, the reagent displaces a remaining X group. In several embodiments, step (ii) is carried out in the presence of base (e.g., DIPEA, 1.5 eq.). In several embodiments, step (ii) is carried out in the presence of a polar aprotic solvent (e.g., THF). In several embodiments, step (ii) is carried out at room temperature or higher (e.g., e.g., 50° C.).

In several embodiments, step (iii) includes providing a reagent (e.g., H-LAₐA_{b}A₁, A₂, or A_{c}A₃ (e.g., 1.0 eq.)) that displaces the last remaining X group (e.g., X₁, X₂, or X₃). In several embodiments, step (iii) is carried out in the presence of base (e.g., NaH or DIPEA, 2.0 eq.). In several embodiments, step (iii) is carried out in the presence of a polar aprotic solvent (e.g., THF). In several embodiments, step (iii) is carried out at room temperature or higher (e.g., e.g., 90° C.).

In several embodiments, a compound of Formula (I) may be prepared through any one of Routes A, B, or C, where any one of steps i, ii, iii, can be omitted. In several embodiments, the variables (and reagents and/or reactants) of FIG. 1B (or FIG. 1A or FIG. 2) may be defined as follows: (i) NHAₐA_{b}A₁, A₂, or A_{c}A₃ (e.g., 1.0 eq.), base (e.g., DIPEA, 1.5 eq.), solvent (e.g., THF, −78° C.-R.T.); (ii) NHAₐA_{b}A₁, A₂, or A_{c}A₃ (1.0 eq.), base (e.g., DIPEA, 1.5 eq.), solvent (e.g., THF), at controlled temperature (e.g., 50° C.). (iii) NHAₐA_{b}A₁, A₂, or A_{c}A₃ (3-5 eq.), NaH or DIPEA (2 eq.), THF, 90° C. In several embodiments, as disclosed elsewhere herein, the method of manufacture may include or omit any step provided in FIG. 1B. In several embodiments, any one of Route A, B, or C may be used. In several embodiments, the method may include some steps from A, B or C of FIG. 1A, but not others.

FIG. 2 provides more specific reaction pathways as disclosed in FIGS. 1A and 1B. In several embodiments, the method of manufacture may include some steps from any of the three routes of FIG. 2, but not others. For example, in several embodiments, the method may include step i, but not ii or iii. In several embodiments, the method may include step i and ii, but not iii. In several embodiments, the method may include steps i, ii, and iii. In several embodiments, the method may include step ii and iii, but not step i. The method may include step ii, but not steps i or iii. The method may include step iii, but not step i or ii.

Several exemplary embodiments pertain to intermediate compounds (e.g., those used to provide compounds of Formula (I)). In several embodiments, the intermediate compounds include those of any one of Formulae (IX^{1,2,3}), (IX^{2,3}), (IX³), (IX^{1,3}), (IX¹), (IX^{1,2}) (IX²), and (IX^{1,2,3}) as shown in FIG. 1A, where X¹ is a halogen.

### Administration and Pharmaceutical Compositions

The compounds are administered at a therapeutically effective dosage. While human dosage levels have yet to be optimized for the compounds described herein, generally, a daily dose may be from about 0.25 mg/kg to about 120 mg/kg or more of body weight, from about 0.5 mg/kg or less to about 70 mg/kg, from about 1.0 mg/kg to about 50 mg/kg of body weight, or from about 1.5 mg/kg to about 10 mg/kg of body weight. Thus, for administration to a 70 kg person, the dosage range would be from about 17 mg per day to about 8000 mg per day, from about 35 mg per day or less to about 7000 mg per day or more, from about 70 mg per day to about 6000 mg per day, from about 100 mg per day to about 5000 mg per day, or from about 200 mg to about 3000 mg per day. The amount of active compound administered will, of course, be dependent on the subject and disease state being treated, the severity of the affliction, the manner and schedule of administration and the judgment of the prescribing physician.

Administration of the compounds disclosed herein or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

The compounds useful as described above can be formulated into pharmaceutical compositions for use in treatment of these conditions. Standard pharmaceutical formulation techniques are used, such as those disclosed in Remington's The Science and Practice of Pharmacy, 21st Ed., Lippincott Williams & Wilkins (2005), incorporated by reference in its entirety. Accordingly, some embodiments include pharmaceutical compositions comprising: (a) a safe and therapeutically effective amount of a compound described herein (including enantiomers, diastereoisomers, tautomers, polymorphs, and solvates thereof), or pharmaceutically acceptable salts thereof; and (b) a pharmaceutically acceptable carrier, diluent, excipient or combination thereof.

In addition to the selected compound useful as described above, some embodiments include compositions containing a pharmaceutically-acceptable carrier. The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, subcutaneous, or other parental routes of administration. In some embodiments, the compositions may be in a form suitable for subcutaneous administration. The skilled artisan will appreciate that oral and nasal compositions comprise compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modem Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carrier suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual dose of the active compounds described herein depends on the specific compound, and on the condition to be treated; the selection of the appropriate dose is well within the knowledge of the skilled artisan.

The compounds and compositions described herein, if desired, may be presented in a pack or dispenser device containing one or more unit dosage forms containing the active ingredient. Such a pack or device may, for example, comprise metal or plastic foil, such as a blister pack, or glass, and rubber stoppers such as in vials. The pack or dispenser device may be accompanied by instructions for administration. Compounds and compositions described herein are formulated in a compatible pharmaceutical carrier may also be prepared, placed in an appropriate container, and labeled for treatment of an indicated condition.

The amount of the compound in a formulation can vary within the full range employed by those skilled in the art. Typically, the formulation will contain, on a weight percent (wt %) basis, from about 0.01 99.99 wt % of a compound of the present technology based on the total formulation, with the balance being one or more suitable pharmaceutical excipients. Preferably, the compound is present at a level of about 1 80 wt %. Representative pharmaceutical formulations are described below.

In several embodiments, a “carboxyl ester”, “carboxylate ester”, or “carboxylic ester” can be used as a pro-drug of “carboxylic acid”.

### Formulation Examples

The following are representative pharmaceutical formulations containing a compound of Formula I.

### Formulation Example 1—Tablet Formulation

The following ingredients are mixed intimately and pressed into single scored tablets.

TABLE 0.1

| Ingredient | Quantity per tablet, mg |
|---|---|
| Compounds disclosed herein | 400 |
| cornstarch | 50 |
| croscarmellose sodium | 25 |
| lactose | 120 |
| magnesium stearate | 5 |

### Formulation Example 2—Capsule Formulation

The following ingredients are mixed intimately and loaded into a hard-shell gelatin capsule.

| Ingredient | Quantity per capsule, mg |
|---|---|
| Compounds disclosed herein | 200 |
| lactose, spray-dried | 148 |
| magnesium stearate | 2 |

### Formulation Example 3—Suspension Formulation

The following ingredients are mixed to form a suspension for oral administration.

| Ingredient | Amount |
|---|---|
| Compounds disclosed herein | 1.0 g |
| fumaric acid | 0.5 g |
| sodium chloride | 2.0 g |
| methyl paraben | 0.15 g |
| propyl paraben | 0.05 g |
| granulated sugar | 25.0 g |
| sorbitol (70% solution) | 13.00 g |
| Veegum K (Vanderbilt Co.) | 1.0 g |
| flavoring | 0.035 mL |
| colorings | 0.5 mg |
| distilled water | q.s. to 100 mL |

### Formulation Example 4—Injectable Formulation

The following ingredients are mixed to form an injectable formulation.

| Ingredient | Amount |
|---|---|
| Compounds disclosed herein | 0.2 mg-20 mg |
| sodium acetate buffer solution, 0.4M | 2.0 mL |
| HCl (1N) or NaOH (1N) | q.s. to suitable pH |
| water (distilled, sterile) | q.s. to 20 mL |

### Formulation Example 5—Suppository Formulation

A suppository of total weight 2.5 g is prepared by mixing the compound of the present technology with Witepsol® H-15 (triglycerides of saturated vegetable fatty acid; Riches-Nelson, Inc., New York), and has the following composition:

| Ingredient | Amount |
|---|---|
| Compounds disclosed herein | 500 mg |
| Witepsol ® H-15 | balance |

### Methods of Treatment

The compounds of Formula I disclosed herein or their tautomers and/or pharmaceutically acceptable salts thereof can effectively act as inhibitors of JMJD3. Some embodiments provide pharmaceutical compositions comprising one or more compounds disclosed herein and a pharmaceutically acceptable excipient.

Some embodiments provide a method of preventing, treating, or ameliorating one or more inflammatory or autoimmune diseases in a subject. In some embodiments, the method includes administering one or more of the compounds disclosed herein to a subject in need thereof. In some embodiments, the method includes administering a pharmaceutically acceptable salt thereof of one or more of the compounds disclosed herein to a subject in need thereof.

Some embodiments provide a method preventing, treating, or ameliorating inflammatory bowel disease (IBD). Some embodiments provide a method preventing, treating, or ameliorating ulcerative colitis, Crohn's disease, atopic dermatitis, psoriasis, systemic erythematosus lupus, atherosclerosis, and Type 1 diabetes. Some embodiments provide a method preventing, treating, or ameliorating graft-versus-host-disease (GVHD), Type 1 Diabetes (T1D), Ulcerative Colitis, Crohn's Disease, Systemic Lupus Erythematosus, Asthma, Rheumatoid Arthritis, multiple sclerosis, encephalomyelitis, Sjogren syndrome, Hashimoto's Thyroiditis, Autoimmune Hepatitis, Behcet's Disease, Castleman disease, Allergic Rhinitis, Eczema, Dressler's Syndrome, Eosinophilic esophagitis, Guillain-Barre Syndrome, Juvenile arthritis, Kawasaki disease, asthma, lung allergies, and atopic dermatitis. In some embodiments, the method includes administering one or more of the compounds disclosed herein to a subject in need thereof. In some embodiments, the method includes administering a pharmaceutically acceptable salt thereof of one or more of the compounds disclosed herein to a subject in need thereof.

In some embodiments, the method of administering one or more of the compounds disclosed herein results in the prevention, treatment, or amelioration, of an inflammatory or autoimmune disease. In some embodiments, the method of administering one or more of the compounds disclosed herein results in the prevention, treatment, or amelioration, of ulcerative colitis, Crohn's disease, atopic dermatitis, psoriasis, systemic erythematosus lupus, atherosclerosis, and Type 1 diabetes. In some embodiments, the method includes administering a pharmaceutically acceptable salt thereof of one or more of the compounds disclosed herein.

JMJD3 inhibitors have been implicated in the progression and/or metastatic potential of several cancer types namely, kidney, breast, prostate, skin, hemopoietic system, myelodysplastic syndrome (MDS), esophageal squamous cell carcinoma, head and neck squamous cell carcinoma (HNSCC), and ovarian cancer. High JMJD3 expression was correlated with shortened overall survival in patients with gastric cancer (GC) and was an independent prognosis predictor. Importantly, JMJD3 inhibitors could reverse the oncogenic effect of JMJD3 overexpression in human GC cell lines. In several embodiments, the disclosed compound is used to treat a cancer. In some embodiments, the cancer is selected from the group consisting of colorectal, gastric, stomach, esophageal, liver, pancreatic, breast, prostate, bladder, renal, ovarian, lung, melanoma, and multiple myeloma. In some embodiments, the cancer is selected from the group consisting of kidney cancer, breast cancer, castration resistant prostate cancer, acute myeloid leukemia (AML). melanoma, Hodgkin's lymphoma (HL), diffuse large B-cell lymphoma (DLBCL), myelodysplastic syndrome (MDS), esophageal squamous cell carcinoma, head and neck squamous cell carcinoma (HNSCC), and ovarian cancer. In some embodiments, the cancer is selected from the group consisting of prostate, skin, hemopoietic system, myclodysplastic syndrome (MDS), esophageal squamous cell carcinoma, head and neck squamous cell carcinoma (I-INSCC), and ovarian cancer. In some embodiments, the method includes administering one or more of the compounds disclosed herein to a subject in need thereof. In some embodiments, the method includes administering a pharmaceutically acceptable salt thereof of one or more of the compounds disclosed herein to a subject in need thereof.

In some embodiments, the method of administering one or more of the compounds disclosed herein results in the inhibition of the activity of JMJD3 in one or more organs of said subject. In several embodiments, inhibiting the activity of JMJD3 suppresses the expression and/or activity of pro-inflammatory signals. In several embodiments, the pro-inflammatory signals may one or more cytokines. In several embodiments, the cytokine includes TNF-alpha (TNFA), interleukin 6 (IL6), interleukin 1beta (IL1B), MCP1 and interleukin 8 (IL8). In some embodiments, the method includes administering a pharmaceutically acceptable salt thereof of one or more of the compounds disclosed herein.

Some embodiments include co-administering a compound, composition, and/or pharmaceutical composition described herein, with an additional medicament. By “co-administration,” it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally. In another embodiment, the agents are administered through different routes, such as one being administered subcutaneously, another being administered orally and another being administered i.v.

The following examples are given for the purpose of illustrating various embodiments of the disclosure and are not meant to limit the present disclosure in any fashion. One skilled in the art will appreciate readily that the present disclosure is well adapted to carry out the objects and obtain the ends and advantages mentioned, as well as those objects, ends and advantages inherent herein. Changes therein and other uses which are encompassed within the spirit of the disclosure as defined by the scope of the claims will occur to those skilled in the art.

### Examples

### Example 1: Synthesis of Triazines

All reactions were carried out under an atmosphere of argon. Reagents and solvents were used from commercial sources without additional purification. Hydrogenation reactions were run under a balloon. Microwave reactions were performed using a CEM Discover SP microwave synthesizer. Sample purification was conducted on a Buchi Pureflash with ELSD purification system using pre-packed commercially available silica gel columns. Thin layer chromatography (TLC) was performed on aluminium plates using Merck Kiesegel 60 F254 (230-400 mesh) fluorescent treated silica which were visualized under ultraviolet light (254 nm), or by staining with potassium permanganate or ninhydrin solution as appropriate. All Nuclear Magnetic Resonance (NMR) spectra were acquired on a Bruker Avance III HD 400 MHz NMR spectrometer; chemical shifts are reported in ppm (δ). HPLC/MS was performed on a Sciex 5500 Qtrap mass spectrometry coupled with Shidmazu Nexera X2 UHPLC using Phenomenex Luna C18 column (50×2.0 mm, 3 μm particle size) via following method: The gradient mobile phase A contains 0.1% formic acid in water and mobile phase B contains 0.1% formic acid in acetonitrile; A/B (95:5) from 0 to 0.9 minutes; to A/B (5:95) from 0.9 to 2.2 minutes; A/B (5:95) from 2.2 to 4.14 minutes; to A/B (95:5) from 4.14 to 4.20 minutes; A/B (95:5) from 4.2 to 6 minutes. The flow rate was 0.4 mL/min and the column temperature maintained at 35° C. and autosampler temperature at 4° C. Ion spray voltage, drying gas temperature, ion source gas 1, and ion source gas 2 settings were 4500V, 500° C., 35V, and 45V with ESI set in positive mode using full scan. All compounds purity was analyzed on Agilent 1260 Infinity II Lab LC Series HPLC (1260 Quatpum, 1260 vial autosampler, ICC column oven, 1260 DAD WR detector). Samples were injected into Phenomenex Synergi Polar RP column (150×4.6 mm, 4 μm, 80 Å). The gradient mobile phase (A: water with 0.1% trifluoroacetic acid, B: acetonitrile with 0.1% trifluoroacetic acid; A/B (99:1) from 0 minute; to A/B (1:99) from 0 to 15 minutes; A/B (1:99) from 15 to 18 minutes; A/B (99:1) from 18 to 18.1 minutes; A/B (99:1) from 18.1 to 20 minutes) pumped at a flow rate of 1 mL/min. UV detector was set to 254 nm with column oven at 35° C. Injection volume was 10 μL, unless otherwise specified. All compounds that were evaluated in biological assay had >90% and animal study had >95% purity.

The compounds were prepared in general by a three-consecutive chloro displacement of the triazine scaffolds as depicted in the general Scheme 1 as shown in FIG. 2. In several embodiments, the conditions in Scheme 1 include the following: (i) NHRₐ, pyrazole, or NR_{b}R_{c} (1.0 eq.), DIPEA, (1.5 eq.), THF, −78° C.-R.T.; (ii) NHRₐ, pyrazole, or NR_{b}R_{c} (1.0 eq.), DIPEA, (1.5 eq.), THF, 50° C. (iii) NHRₐ, pyrazole, or NR_{b}R_{c} (3-5 eq.), NaH or DIPEA (2 eq.), THF, 90° C.

To investigate a pyrazole-triazine core toward inhibiting the activity of JMJD3, 6-(1H-pyrazol-1-yl)-1,3,5-triazine analogues were synthesized from commercially cyanic chloride. The structure activity relationship (SAR) of 2,4-substituted of the 6-(1H-pyrazol-1-yl)-1,3,5-triazine was studied with various groups using computation docking to further enhance the potency. All structures disclosed herein were tested by computational molecular docking using the available software SeeSAR v.10. Using a published crystal structure data of compound JMJD3 inhibitor bound to JMJD3 protein (6G8F-GSK-J1) affinity binding was estimated. Data produced using the JMJD3 protein was used to evaluate compounds with good affinity to the JMJD3 protein binding pocket. Each of those compounds is disclosed herein.

The below provide representative methods for preparing the compounds disclosed herein. In view of this guidance and the guidance provided by the remained of this disclosure, those skilled in the art will be able to prepare each structure disclosed herein.

### Example 2: Synthesis of Ethyl 3-((4,6-dichloro-1,3,5-triazin-2-yl)amino)propanoate (INT 1)

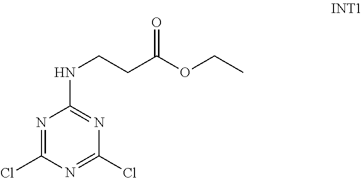 [SMILES: CCOC(=O)CCNc1nc(Cl)nc(Cl)n1]

To a solution cyanuric chloride (1.00 g, 5.42 mmol) in anhydrous THF (20 mL) cooled to −78° C. under argon atmosphere was added β-alanine esther hydrochloride (0.83 g, 5.42 mmol) and stirred for 5 min. prior adding DIPEA (1.05 g, 8.13 mmol). The reaction mixtures were stirred for addition 1 hour and then slowly equilibrate to 0° C. at which it was maintained overnight. The reaction was extracted with ethyl acetate (2×50 mL) and washed with sat. NaHCO₃ followed with brine. The combined organic layers were dried over anhydrous Na₂SO₄ and concentrated in vacuo. The residue was purified by Buchi Pureflash chromatography over silica gel cartridge (80 g) with 100% CH₂Cl₂ to give ethyl 3-((4,6-dichloro-1,3,5-triazin-2-yl)amino)propanoate (1.09 g, 76%) as a white solid. ¹H NMR (400 MHz, CDCl₃): δ 6.45 (bs, 1H), 4.15 (q, 2H, J=8.0 Hz), 3.74 (q, 2H, J=8.0 Hz), 2.61 (t, 2H, J=8.0 Hz), 1.25 (t, 3H, J=8.0 Hz). MS (ESI): Calcd. for C₈H₁₀Cl₂N₄O₂: 265, found 265 (M)⁺

### Example 3: Synthesis of Ethyl 3-((4-chloro-6-(1H-pyrazol-1-yl)-1,3,5-triazin-2-yl)amino)propanoate (INT 2)

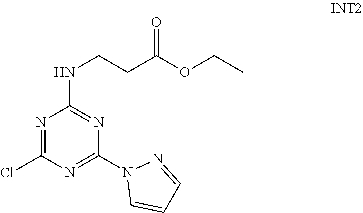 [SMILES: CCOC(=O)CCNc1nc(Cl)nc(-n2cccn2)n1]

To a solution ethyl 3-((4,6-dichloro-1,3,5-triazin-2-yl)amino)propanoate (0.20 g, 0.75 mmol) in anhydrous THF (6 mL) was DIPEA (0.16 g, 1.51 mmol) and pyrazole (0.05 g, 0.75 mmol) under argon atmosphere. The sealed tube was heated to 50° C. for 4 days. The cooled reaction was extracted with 8:2 dichloromethane/isopropanol mixture (3×20 mL) and washed with sat. NaHCO₃ followed by brine. The combined organic layers were dried over anhydrous Na₂SO₄ and concentrated in vacuo. The residue was purified by Buchi Pureflash chromatography over silica gel cartridge (40 g) with 97:3 CH₂Cl₂:MeOH with 0.7N ammonia to give ethyl 3-((4-chloro-6-(1H-pyrazol-1-yl)-1,3,5-triazin-2-yl)amino)propanoate (0.13 g, 60%) as a white solid. ¹H NMR (400 MHz, CDCl₃): δ 8.50 (dd, 1H, J=12.0, 4.0 Hz), 7.83 (dd, 1H, J=16.0, 4.0 Hz), 6.51 (bs, 1H), 6.48 (m, 1H), 4.16 (q, 2H, J=8.0 Hz), 8.81 (q, 2H, J=8.0 Hz), 2.63 (t, 2H, J=8.0 Hz), 1.25 (t, 3H, J=8.0 Hz). MS (ESI): Calcd. for C₁₁H₁₃ClN₆O₂: 296, found 297 (M)⁺ and 319 (M+23)⁺

### Example 4: Synthesis of Ethyl 3-((4-(1H-pyrazol-1-yl)-6-(1,2,4,5-tetrahydro-3H-benzo[d]azepin-3-yl)-1,3,5-triazin-2-yl)amino)propanoate (Compound 1)

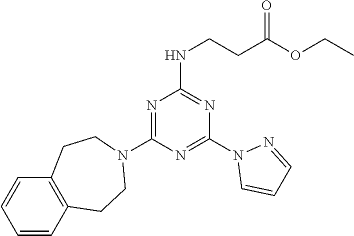 [SMILES: CCOC(=O)CCNc1nc(N2CCc3ccccc3CC2)nc(-n2cccn2)n1]

To a solution ethyl 3-((4-chloro-6-(1H-pyrazol-1-yl)-1,3,5-triazin-2-yl)amino)propanoate (0.10 g, 0.34 mmol) in anhydrous THF (2 mL) was DIPEA (0.11 g, 0.84 mmol) and 2,3,4,5-tetrahydro-1H-3-benzazepine (0.99 g, 0.67 mmol) under argon atmosphere. The sealed tube was heated to 50° C. overnight. The cooled reaction was extracted with 8:2 dichloromethane/isopropanol mixture (3×20 mL) and washed with sat. NaHCO₃ followed by brine. The combined organic layers were dried over anhydrous Na₂SO₄ and concentrated in vacuo. The residue was purified by Buchi Pureflash chromatography over silica gel cartridge (40 g) with 97:3 CH₂Cl₂:MeOH with 0.7N ammonia to give ethyl 3-((4-(1H-pyrazol-1-yl)-6-(1,2,4,5-tetrahydro-3H-benzo[d]azepin-3-yl)-1,3,5-triazin-2-yl)amino)propanoate (0.12 g, 86%) as a light yellow solid. ¹H NMR (400 MHz, CDCl₃): δ 8.50 (dd, 1H, J=12.0, 4.0 Hz), 7.75 (dd, 1H, J=12.0, 4.0 Hz), 7.14 (s, 4H), 6.41 (m, 1H), 5.80 (t, 1H, J=4.0 Hz), 4.15 (q, 2H, J=8.0 Hz), 4.02 (m, 4H), 3.76 (q, 2H, J=8.0 Hz), 2.97 (m, 4H), 2.63 (t, 2H, J=8.0 Hz), 1.25 (t, 3H, J=8.0 Hz). MS (ESI): Calcd. for C₂₁H₂₅N₇O₂: 407, found 408 (M)⁺

### Example 5: Synthesis of Ethyl 3-((4,6-di(1H-pyrazol-1-yl)-1,3,5-triazin-2-yl)amino)propanoate (Compound 2)

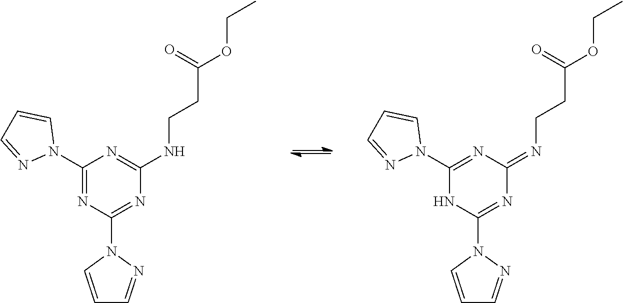 [SMILES: CCOC(=O)CCNc1nc(-n2cccn2)nc(-n2cccn2)n1 ; CCOC(=O)CCN=c1nc(-n2cccn2)[nH]c(-n2cccn2)n1]

To a solution ethyl 3-((4,6-dichloro-1,3,5-triazin-2-yl)amino)propanoate (0.15 g, 0.57 mmol) in anhydrous THF (3 mL) was DIPEA (0.22 g, 1.70 mmol) and pyrazole (0.19 g, 2.82 mmol) under argon atmosphere. The sealed tube was heated to 90° C. for 4 days. The cooled reaction was removed of its solvent and immediately purified by Buchi Pureflash chromatography over silica gel cartridge (40 g) with 95:5 CH₂Cl₂:MeOH with 0.7N ammonia to give ethyl 3-((4,6-di(1H-pyrazol-1-yl)-1,3,5-triazin-2-yl)amino)propanoate (0.18 g, 99%) as an off-white solid. ¹H NMR (400 MHz, CDCl₃): δ 8.63 (dd, 2H, J=12.0, 4.0 Hz), 7.84 (dd, 2H, J=12.0, 2.0 Hz), 7.33 (bs, 0.5H), 6.49 (m, 2H), 6.33 (t, 0.5H, J=4.0 Hz), 4.15 (q, 2H, J=12.0, 4.0 Hz), 3.92 (q, 2H, J=12.0, 4.0 Hz), 2.67 (t, 2H, J=8.0 Hz), 1.24 (t, 3H, J=8.0 Hz). MS (ESI): Calcd. for C₁₄H₁₆N₈O₂: 328, found 329 (M+1)⁺ and 351 (M+23)⁺

### Example 6: Synthesis of 1-(4-Chloro-6-(1H-pyrazol-1-yl)-1,3,5-triazin-2-yl)-1H-indole (INT3)

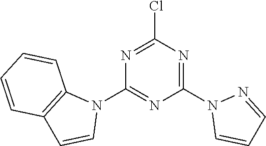 [SMILES: Clc1nc(-n2cccn2)nc(-n2ccc3ccccc32)n1]

To a solution indole (2.54 g, 21.69 mmol) in anhydrous THF (30 mL) under ice bath was added 60% sodium hydride (1.08 g, 27.11 mmol) in portion and stirred until no bubble evolves. In a separate flash was charged with cyanic chloride (4.00 g, 21.69 mmol) in anhydrous THF (30 mL) under argon cooled to −78° C. The solution of indole/sodium hydride was slowly cannula over a period of 20 min under −78° C. The mixtures were stirred for 90 minutes and monitored by LCMS (mass of 265). After completion, DIPEA (5.61 g, 43.38 mmol) and pyrazole (1.55 g, 22.78 mmol) added, then equilibrate to 0° C. with stirring for 2 hours. The mixtures were than allowed to stir at room temperature for 2 days. After completion, the mixtures were filtered through a pad of celite washing thoroughly with ethyl acetate and dichloromethane. The organic layer was washed with brine (400 mL) and extracted with ethyl acetate (2×100 mL). The combined organic layers were dried over anhydrous Na₂SO₄, filtered and concentrated in vacuo. The residue was purified by Buchi Pureflash chromatography over silica gel cartridge (120 g) with 100% dichloromethane to give 1-(4-chloro-6-(1H-pyrazol-1-yl)-1,3,5-triazin-2-yl)-1H-indole (1.30 g, 20%) as a light powder yellow solid. ¹H-NMR (400 MHz, DMSO-d₆): δ 8.87 (dd, 1H, J=48, 4.0 Hz), 8.83 (dd, 1H, J=44, 8.0 Hz), 8.33 (dd, 1H, J=28, 2.0 Hz), 8.09 (dd, 1H, J=16.0, 4.0 Hz), 7.68 (dd, 1H, J=12.0, 4.0 Hz), 7.47-7.26 (m, 2H), 6.91 (ddd, 1H, J=36, 4.0, 2.0 Hz), 6.77 (m, 1H). MS (ESI): Calcd. for C₁₄H₉ClN₆: 296, found 297 (M+1)⁺

### Example 7: Synthesis of 4-(1H-indol-1-yl)-N-(2-methoxyethyl)-6-(1H-pyrazol-1-yl)-1,3,5-triazin-2-amine (Compound 89)

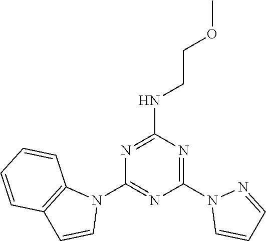 [SMILES: COCCNc1nc(-n2cccn2)nc(-n2ccc3ccccc32)n1]

To a solution ethyl 1-(4-chloro-6-(1H-pyrazol-1-yl)-1,3,5-triazin-2-yl)-1H-indole (0.10 g, 0.34 mmol) in anhydrous THF (2 mL) was DIPEA (0.09 g, 0.67 mmol) and 2-methoxyethylamine (0.13 g, 1.69 mmol) under argon atmosphere. The sealed tube was heated to 50° C. for 20 h. The cooled reaction was extracted with 8:2 dichloromethane/isopropanol mixture (3×20 mL) and washed with sat. NaHCO₃ followed by brine. The combined organic layers were dried over anhydrous Na₂SO₄ and concentrated in vacuo. The residue was purified by Buchi Pureflash chromatography over silica gel cartridge (40 g) with 100% ethyl acetate to give 4-(1H-indol-1-yl)-N-(2-methoxyethyl)-6-(1H-pyrazol-1-yl)-1,3,5-triazin-2-amine (0.04 g, 39%) as a white solid. ¹H-NMR (400 MHz, DMSO-d₆): δ 8.88 (dd, 1H, J=64.0, 8.0 Hz), 8.8 (dd, 1H, J=28.0, 4.0 Hz), 8.63 (dt, 1H, J=16.0, 4.0 Hz), 8.35 (dd, 1H, J=32.0, 4.0 Hz), 7.95 (dd, 1H, J=4.0, 2.0 Hz), 7.65 (m, 1H), 7.36 (m, 1H), 7.25 (m, 1H), 6.83 (dd, 1H, J=8.0, 4.0 Hz, 6.66 (m, 1H), 3.67 (m, 2H), 3.59 (m, 2H), 3.31 (s, 3H). MS (ESI): Calcd. for C₁₇H₁₇N₇O: 335, found 336 (M)⁺ and 358 (M+Na)⁺

### Example 8: Synthesis of 1-(4-(Piperazin-1-yl)-6-(1H-pyrazol-1-yl)-1,3,5-triazin-2-yl)-1H-indole (Compound 90)

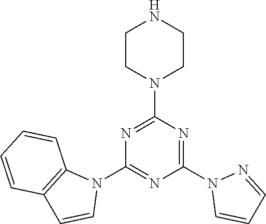 [SMILES: c1ccc2c(c1)ccn2-c1nc(N2CCNCC2)nc(-n2cccn2)n1]

To a solution ethyl 1-(4-chloro-6-(1H-pyrazol-1-yl)-1,3,5-triazin-2-yl)-1H-indole (0.10 g, 0.34 mmol) in anhydrous THF (2 mL) was piperazine (0.17 g, 2.02 mmol) under argon atmosphere. The sealed tube was heated to 50° C. for 20 h. The cooled reaction was extracted with 8:2 dichloromethane/isopropanol mixture (3×20 mL) and washed with sat. NaHCO₃ (2×20 mL) followed by brine. The combined organic layers were dried over anhydrous Na₂SO₄ and concentrated in vacuo. The product was precipitate in dichloromethane and collected by filtration to give 1-(4-(piperazin-1-yl)-6-(1H-pyrazol-1-yl)-1,3,5-triazin-2-yl)-1H-indole (0.05 g, 47%) as a white solid. ¹H-NMR (400 MHz, DMSO-d₆/MeOD): δ 8.72 (s, 1H), 8.37 (d, 1H, J=4.0 Hz), 7.87 (s, 1H), 7.60 (d, 1H, J=8.0 Hz), 7.37 (m, 1H), 7.25 (m, 1H), 6.72 (d, 1H, J=4.0 Hz), 6.59 (d, 1H, J=0.2 Hz) 4.27 (bs, 2H), 4.23 (bs, 2H), 1.22 (bs, 4H), 1.11 (d, 1H, J=8.0 Hz). MS (ESI): Calcd. for C₁₈H₁₈N₈: 346, found 347 (M)⁺

### Example 9: Synthesis of 1-(4-(Cyclopentyloxy)-6-(1H-pyrazol-1-yl)-1,3,5-triazin-2-yl)-1H-indole (Compound 95)

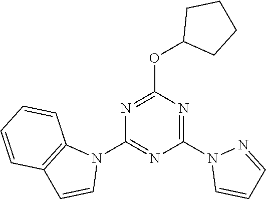 [SMILES: c1ccc2c(c1)ccn2-c1nc(OC2CCCC2)nc(-n2cccn2)n1]

To a solution ethyl 1-(4-chloro-6-(1H-pyrazol-1-yl)-1,3,5-triazin-2-yl)-1H-indole (0.10 g, 0.34 mmol) in anhydrous THF (2 mL) cooled to −78° C. under argon was added pentanol (0.04 g, 0.40 mmol) and potassium tert-butoxide (0.04 g, 0.37 mmol) stirred for 25 min. Then, the mixture was equilibrated to room temperature and stirred overnight (24 h). The reaction was quenched with water then extracted with 8:2 dichloromethane/isopropanol mixture (3×20 mL) and washed with brine. The combined organic layers were dried over anhydrous Na₂SO₄ and concentrated in vacuo. The residue was purified by Buchi Pureflash chromatography over silica gel cartridge (24 g) with 100% dichloromethane to give 1-(4-(cyclopentyloxy)-6-(1H-pyrazol-1-yl)-1,3,5-triazin-2-yl)-1H-indole (0.05 g, 42%) as a white solid. ¹H-NMR (400 MHz, DMSO-d₆): δ 8.85 (m, 2H), 8.35 (d, 1H, J=4.0 Hz), 8.03 (d, 1H, J=0.5 Hz), 7.68 (d, 1H, J=8.0 Hz), 7.42 (dt, 1H, J=8.0, 0.5 Hz), 7.30 (dt, 1H, J=8.0, 0.5 Hz), 6.89 (dd, 1H, J=4.0, 0.2 Hz), 6.71 (dd, 1H, J=4.0, 4.0, Hz), 5.66 (m, 1H), 2.08 (m, 2H), 1.92 (m, 2H), 1.79 (m, 2H), 1.67 (m, 2H). MS (ESI): Calcd. for C₁₉H₁₈N₆O: 346, found 279 (hydrolyzed —OH product) unstable under acidic LCMS condition.

### Example 10: Synthesis of Ethyl 3-((4-(1H-indol-1-yl)-6-(1H-pyrazol-1-yl)-1,3,5-triazin-2-yl)amino)propanoate (Compound 6)

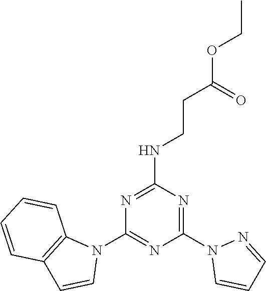 [SMILES: CCOC(=O)CCNc1nc(-n2cccn2)nc(-n2ccc3ccccc32)n1]

To a solution ethyl 1-(4-chloro-6-(1H-pyrazol-1-yl)-1,3,5-triazin-2-yl)-1H-indole (0.10 g, 0.34 mmol) in anhydrous THF (2 mL) was DIPEA (0.43 g, 3.37 mmol) and beta-alanine ethyl ester hydrochloride (0.21 g, 1.35 mmol) under argon atmosphere. The sealed tube was heated to 50° C. for 20 h. The cooled reaction was extracted with 8:2 dichloromethane/isopropanol mixture (3×20 mL) and washed with sat. NaHCO₃ followed by brine. The combined organic layers were dried over anhydrous Na₂SO₄ and concentrated in vacuo. The product was precipitate in minimum amount of methanol and collected by filtration to give ethyl 3-((4-(1H-indol-1-yl)-6-(1H-pyrazol-1-yl)-1,3,5-triazin-2-yl)amino)propanoate (0.10 g, 77%) as a white solid. ¹H-NMR (400 MHz, DMSO-d₆): δ 8.87 (dd, 1H, J=48.0, 8.0 Hz), 8.80 (dd, 1H, J=20.0, 4.0 Hz), 8.63 (dt, 1H, J=32.0, 4.0 Hz), 8.34 (dd, 1H, J=36.0, 4.0 Hz), 7.94 (ddd, 1H, J=16.0, 2.0, 2.0 Hz), 7.66 (t, 1H, J=8.0 Hz), 7.36 (m, 1H), 7.26 (m, 1H), 6.84 (ddd, 1H, J=8.0, 4.0, 2.0 Hz), 6.66 (m, 1H), 4.07 (q, 2H, J=8.0 Hz), 3.78-3.69 (m, 2H), 2.71 (m, 2H), 1.18 (m, 3H). MS (ESI): Calcd. for C₁₉H₁₉N₇O₂: 377, found 378 (M)⁺ and 400 (M+Na)⁺

### Example 11: Synthesis of 3-((4-(1H-Indol-1-yl)-6-(1H-pyrazol-1-yl)-1,3,5-triazin-2-yl)amino)propanamide (Compound 91)

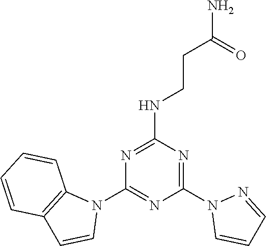 [SMILES: NC(=O)CCNc1nc(-n2cccn2)nc(-n2ccc3ccccc32)n1]

To a solution ethyl 1-(4-chloro-6-(1H-pyrazol-1-yl)-1,3,5-triazin-2-yl)-1H-indole (0.10 g, 0.34 mmol) in anhydrous THF (2 mL) was DIPEA (0.43 g, 3.37 mmol) and 3-amino-propionamide hydrochloride (0.21 g, 1.35 mmol) under argon atmosphere. The sealed tube was heated to 50° C. for 20 h. The cooled reaction was extracted with 8:2 dichloromethane/isopropanol mixture (3×20 mL) and washed with sat. NaHCO₃ followed by brine. The combined organic layers were dried over anhydrous Na₂SO₄ and concentrated in vacuo. The product was precipitate in minimum amount of dichloromethane and collected by filtration to give 3-((4-(1H-indol-1-yl)-6-(1H-pyrazol-1-yl)-1,3,5-triazin-2-yl)amino)propanamide (0.11 g, 93%) as a white solid. ¹H-NMR (400 MHz, DMSO-d₆): δ 8.90 (dd, 1H, J=44.0, 8.0 Hz), 8.80 (dd, 1H, J=28.0, 4.0 Hz), 8.56 (dt, 1H, J=16.0, 4.0 Hz), 8.35 (dd, 1H, J=40.0, 4.0 Hz), 7.94 (ddd, 1H J=8.0. 2.0, 2.0 Hz) 7.65 (m, 1H), 7.41 (bs, 1H), 7.35 (m, 1H), 7.26 (m, 1H), 6.91 (bs, 1H), 6.83 (ddd, 1H, J=8.0, 4.0, 2.0 Hz, 6.66 (m, 1H), 3.68 (m, 2H), 2.49 (m, 2H). MS (ESI): Calcd. for C₁₇H₁₆N₈O: 348, found 349 (M)⁺ and 371 (M+Na)⁺

### Example 12: Synthesis of 4-(1H-Indol-1-yl)-N-(3-methoxypropyl)-6-(1H-pyrazol-1-yl)-1,3,5-triazin-2-amine (Compound 97)

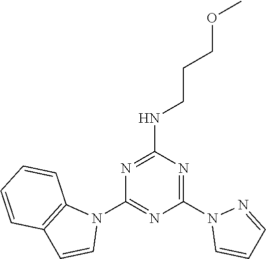 [SMILES: COCCCNc1nc(-n2cccn2)nc(-n2ccc3ccccc32)n1]

To a solution ethyl 1-(4-chloro-6-(1H-pyrazol-1-yl)-1,3,5-triazin-2-yl)-1H-indole (0.10 g, 0.34 mmol) in anhydrous THF (2 mL) was DIPEA (0.09 g, 0.67 mmol) and 3-methoxypropylamine (0.1 g, 1.72 mmol) under argon atmosphere. The sealed tube was heated to 50° C. for 20 h. The cooled reaction was extracted with 8:2 dichloromethane/isopropanol mixture (3×20 mL) and washed with sat. NaHCO₃ followed by brine. The combined organic layers were dried over anhydrous Na₂SO₄ and concentrated in vacuo. The residue was purified by Buchi Pureflash chromatography over silica gel cartridge (40 g) with 100% ethyl acetate to give 4-(1H-indol-1-yl)-N-(3-methoxypropyl)-6-(1H-pyrazol-1-yl)-1,3,5-triazin-2-amine (0.06 g, 53%) as a white solid. ¹H-NMR (400 MHz, DMSO-d₆): δ 8.89 (dd, 1H, J=52.0 8.0 Hz), 8.80 (dd, 1H, J=12.0, 4.0 Hz), 8.60 (dt, 1H, J=40.0, 8.0 Hz, 8.34 (dd, 1H, J=36.0, 4.0 Hz), 7.94 (ddd, 1H, J=8.0, 2.0, 2.0 Hz), 7.65 (dd, 1H, J=8.0, 4.0 Hz), 7.36 (m, 1H), 7.26 (m, 1H), 6.82 (ddd, 1H, J=8.0, 4.0, 2.0 Hz), 6.66 (m, 1H), 3.54 (m, 2H), 3.45 (q, 2H, J=4.0 Hz), 3.26 (s, 3H), 1.87 (m, 2H). MS (ESI): Calcd. for C₁₈H₁₉N₇O: 349, found 350 (M)⁺ and 372 (M+Na)⁺

### Example 14: Synthesis of 2-(Azetidin-1-yl)-4-(3,5-difluorophenyl)-6-(1H-pyrazol-1-yl)-1,3,5-triazine (Compound 93)

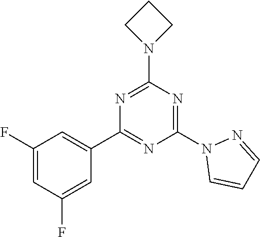 [SMILES: Fc1cc(F)cc(-c2nc(N3CCC3)nc(-n3cccn3)n2)c1]

To a solution 2,4-dichloro-6-(3,5-difluorophenyl)-1,3,5-triazine (0.10 g, 0.38 mmol) in anhydrous THF (2 mL) was pyrazole (0.03 g, 0.40 mmol) and DIPEA (0.10 g, 0.76 mmol) at 0° C. under argon atmosphere. The mixtures were kept in 4° C. refrigerator overnight. After completion by monitoring with LCMS (mass of 294), then azetidine (0.04 g, 0.42 mmol) was added via syringe. The sealed tube was stirred to room temperature for an hour and precipitation formed. The cooled reaction was extracted with 8:2 dichloromethane/isopropanol mixture (3×20 mL) and washed with brine. The combined organic layers were dried over anhydrous Na₂SO₄ and concentrated in vacuo. The residue was purified by Buchi Pureflash chromatography over silica gel cartridge (40 g) with 100% ethyl acetate to give 2-(azetidin-1-yl)-4-(3,5-difluorophenyl)-6-(1H-pyrazol-1-yl)-1,3,5-triazine (0.06 g, 50%) as a white solid. ¹H-NMR (400 MHz, DMSO-d₆): δ 8.89 (dd, 1H, J=8.0, 1.0 Hz), 8.04 (m, 2H), 7.90 (dd, 1H, J=4.0, 1.0 Hz), 7.55 (tt, 1H, J=8.0, 4.0 Hz), 6.62 (dd, 1H, J=4.0, 2.0 Hz), 4.26 (dt, 4H, J=20.0, 8.0 Hz), 2.39 (p, 2H, J=8.0 Hz). MS (ESI): Calcd. for C₁₅H₁₂F₂N₆: 314, found 315 (M)⁺ and 337 (M+Na)⁺

### Example 15: Synthesis of 2-(3,5-Difluorophenyl)-4-(4-methylpiperazin-1-yl)-6-(1H-pyrazol-1-yl)-1,3,5-triazine (Compound 80)

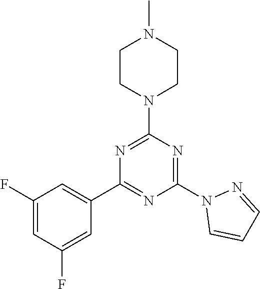 [SMILES: CN1CCN(c2nc(-c3cc(F)cc(F)c3)nc(-n3cccn3)n2)CC1]

To a solution 2,4-dichloro-6-(3,5-difluorophenyl)-1,3,5-triazine (0.10 g, 0.38 mmol) in anhydrous THF (2 mL) was pyrazole (0.03 g, 0.40 mmol) and DIPEA (0.10 g, 0.76 mmol) at 0° C. under argon atmosphere. The mixtures were kept in 4° C. refrigerator overnight. After completion by monitoring with LCMS (mass of 294), then 1-methylpiperazine (0.05 g, 0.46 mmol) was added via syringe. The sealed tube was stirred to room temperature for an hour and precipitation formed. The cooled reaction was extracted with 8:2 dichloromethane/isopropanol mixture (3×20 mL) and washed with sat. NaHCO₃ followed brine. The combined organic layers were dried over anhydrous Na₂SO₄ and concentrated in vacuo. The residue was purified by Buchi Pureflash chromatography over silica gel cartridge (24 g) with 95:5 CH₂Cl₂:MeOH to give 2-(3,5-difluorophenyl)-4-(4-methylpiperazin-1-yl)-6-(1H-pyrazol-1-yl)-1,3,5-triazine (0.11 g, 81%) as a white solid. ¹H-NMR (400 MHz, DMSO-d₆): δ 8.95 (dd, 1H, J=4.0, 1.0 Hz), 8.12 (m, 2H), 7.91 (dd, 1H, J=2.0, 1.0 Hz), 7.56 (tt, 1H, J=8.0, 4.0 Hz), 6.64 (dd, 1H, J=4.0, 2.0 Hz), 4.02 (bs, 2H), 3.94 (bs, 2H), 2.46 (m, 4H), 2.26 (s, 3H). MS (ESI): Calcd. for C₁₇H₁₇F₂N₇: 357, found 358 (M)⁺ and 380 (M+Na)⁺

### Example 16: Synthesis of Ethyl 3-((4-(3,5-difluorophenyl)-6-(1H-pyrazol-1-yl)-1,3,5-triazin-2-yl)amino)propanoate (Compound 8)

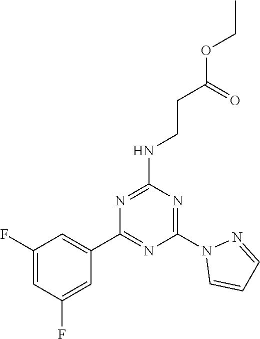 [SMILES: CCOC(=O)CCNc1nc(-c2cc(F)cc(F)c2)nc(-n2cccn2)n1]

To a solution 2,4-dichloro-6-(3,5-difluorophenyl)-1,3,5-triazine (0.10 g, 0.38 mmol) in anhydrous THF (2 mL) was pyrazole (0.03 g, 0.40 mmol) and DIPEA (0.17 g, 1.34 mmol) at 0° C. under argon atmosphere. The mixtures were kept in 4° C. refrigerator overnight. After completion by monitoring with LCMS (mass of 294), then beta-alanine ethyl ester hydrochloride (0.07 g, 0.46 mmol) was added via syringe. The sealed tube was stirred to room temperature for an hour and precipitation formed. The cooled reaction was extracted with 8:2 dichloromethane/isopropanol mixture (3×20 mL) and washed with sat. NaHCO₃ followed brine. The combined organic layers were dried over anhydrous Na₂SO₄ and concentrated in vacuo. The residue was purified by Buchi Pureflash chromatography over silica gel cartridge (24 g) with 100% ethyl acetate to give ethyl 3-((4-(3,5-difluorophenyl)-6-(1H-pyrazol-1-yl)-1,3,5-triazin-2-yl)amino)propanoate (0.06 g, 42%) as a white solid. ¹H-NMR (400 MHz, DMSO-d₆): δ 8.91 (dt, 1H, J=4.0, 1.0 Hz), 8.67 (dt, 1H, J=52.0, 4.0 Hz), 8.13 (m, 1H), 8.02 (m, 1H), 7.91 (dq, 1H, J=12.0, 4.0 Hz), 7.57 (m, 1H), 6.64 (dq, 1H, J=4.0, 1.0 Hz), 4.05 (m, 2H), 3.72 (m, 2H), 2.67 (q, 2H, J=8.0 Hz), 1.15 (t, 3H, J=8.0 Hz). MS (ESI): Calcd. for C₁₇H₁₆F₂N₆O₂: 374, found 375 (M)⁺ and 397 (M+Na)⁺

### Example 13: Synthesis of 4-(4-(3,5-Difluorophenyl)-6-(1H-pyrazol-1-yl)-1,3,5-triazin-2-yl)morpholine (Compound 92)

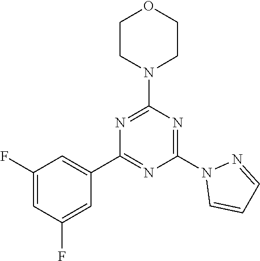 [SMILES: Fc1cc(F)cc(-c2nc(N3CCOCC3)nc(-n3cccn3)n2)c1]

To a solution 2,4-dichloro-6-(3,5-difluorophenyl)-1,3,5-triazine (0.10 g, 0.38 mmol) in anhydrous THF (2 mL) was pyrazole (0.03 g, 0.40 mmol) and DIPEA (0.10 g, 0.76 mmol) at 0° C. under argon atmosphere. The mixtures were kept in 4° C. refrigerator overnight. After completion by monitoring with LCMS (mass of 294), then morpholine (0.04 g, 0.42 mmol) was added via syringe. The sealed tube was stirred to room temperature for an hour and precipitation formed. The cooled reaction was extracted with 8:2 dichloromethane/isopropanol mixture (3×20 mL) and washed with sat. NaHCO₃ followed by brine. The combined organic layers were dried over anhydrous Na₂SO₄ and concentrated in vacuo. The residue was purified by Buchi Pureflash chromatography over silica gel cartridge (40 g) with 95:5 CH₂Cl₂:MeOH to give 4-(4-(3,5-difluorophenyl)-6-(1H-pyrazol-1-yl)-1,3,5-triazin-2-yl)morpholine (0.11 g, 82%) as a white solid. ¹H-NMR (400 MHz, DMSO-d₆): δ 8.95 (dd, 1H, J=4.0, 2.0 Hz), 8.13 (m, 2H), 7.91 (dd, 1H, J=4.0, 2.0 Hz), 7.56 (tt, 1H, J=8.0, 4.0 Hz), 6.64 (dd, 1H, J=4.0, 2.0 Hz), 4.03 (t, 2H, J=4.0 Hz), 3.94 (t, 2H, J=4.0 Hz), 3.73 (t, 4H, J=4.0 Hz). MS (ESI): Calcd. for C₁₆H₁₄F₂N₆O: 344, found 345 (M)⁺ and 367 (M+Na)⁺

### Example 17: Synthesis of 4-(4-(1H-Indol-1-yl)-6-(1H-pyrazol-1-yl)-1,3,5-triazin-2-yl)morpholine (Compound 94)

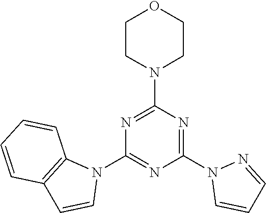 [SMILES: c1ccc2c(c1)ccn2-c1nc(N2CCOCC2)nc(-n2cccn2)n1]

To a solution ethyl 1-(4-chloro-6-(1H-pyrazol-1-yl)-1,3,5-triazin-2-yl)-1H-indole (0.10 g, 0.34 mmol) in anhydrous THF (2 mL) was DIPEA (0.09 g, 0.67 mmol) and morpholine (0.12 g, 1.35 mmol) under argon atmosphere. The reaction was stirred at room temperature for 3 h. The reaction was then extracted with 8:2 dichloromethane/isopropanol mixture (3×20 mL) and washed with sat. NaHCO₃ followed by brine. The combined organic layers were dried over anhydrous Na₂SO₄ and concentrated in vacuo. The residue was purified by Buchi Pureflash chromatography over silica gel cartridge (12 g) with 100% ethyl acetate to give 4-(4-(1H-indol-1-yl)-6-(1H-pyrazol-1-yl)-1,3,5-triazin-2-yl)morpholine (0.11 g, 98%) as a white solid. ¹H-NMR (400 MHz, DMSO-d₆): δ 8.86 (dd, 1H, J=4.0, 1.0 Hz), 8.80 (d, 1H, J=12.0 Hz), 8.38 (d, 1H, J=4.0 Hz), 7.97 (dd, 1H, J=4.0, 1.0 Hz), 7.66 (d, 1H, J=8.0 Hz), 7.38 (dt, 1H, J=8.0, 1.0 Hz), 7.26 (dt, 1H, J=8.0, 0.5 Hz), 6.67 (dd, 1H, J=4.0, 1.0 Hz), 3.97 (m, 4H), 3.75 (m, 4H). MS (ESI): Calcd. for C₁₈H₁₇N₇O: 347, found 348 (M)⁺

### Example 18: Synthesis of 3-((4-(3,5-Difluorophenyl)-6-(1H-pyrazol-1-yl)-1,3,5-triazin-2-yl)amino)propanamide (Compound 96)

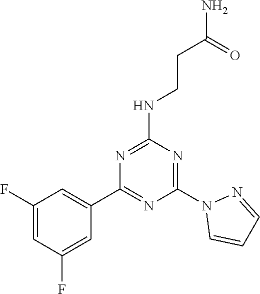 [SMILES: NC(=O)CCNc1nc(-c2cc(F)cc(F)c2)nc(-n2cccn2)n1]

To a solution 2,4-dichloro-6-(3,5-difluorophenyl)-1,3,5-triazine (0.10 g, 0.38 mmol) in anhydrous THF (2 mL) was pyrazole (0.03 g, 0.40 mmol) and DIPEA (0.25 g, 1.91 mmol) at 0° C. under argon atmosphere. The mixtures were kept in 4° C. refrigerator overnight. After completion by monitoring with LCMS (mass of 294), then 3-amino-propionamide hydrochloride (0.04 g, 0.42 mmol) and anhydrous isopropanol (2 mL) was added. The sealed tube was heated at 50° C. for 3 hours and precipitation formed. The cooled reaction was extracted with 8:2 dichloromethane/isopropanol mixture (3×20 mL) and washed with sat. NaHCO₃ followed by brine. The combined organic layers were dried over anhydrous Na₂SO₄ and concentrated in vacuo. The product was precipitate in minimum amount of dichloromethane and collected by filtration to give 3-((4-(3,5-difluorophenyl)-6-(1H-pyrazol-1-yl)-1,3,5-triazin-2-yl)amino)propanamide (0.89 g, 68%) as a white solid. ¹H-NMR (400 MHz, DMSO-d₆): δ 8.90 (ddd, 1H, J=8.0, 2.0, 0.5 Hz), 8.60 (dt, 1H, 36.0, 4.0 Hz), 8.13 (dd, 1H, J=8.0, 4.0 Hz), 8.03 (dd, 1H, J=8.0, 4.0 Hz), 7.90 (ddd, 1H, J=8.0, 1.0, 0.5 Hz), 7.57 (m, 1H), 7.40 (bs, 1H), 6.89 (bs, 1H), 6.63 (dq, 1H, J=8.0, 1.0 Hz), 3.66 (m, 2H), 2.45 (t, 2H, J=8.0 Hz). MS (ESI): Calcd. for C₁₅H₁₃F₂N₇O: 345, found 346 (M)⁺

### Example 19: Synthesis of 8-(4-(1H-Indol-1-yl)-6-(1H-pyrazol-1-yl)-1,3,5-triazin-2-yl)-2-oxa-5,8-diazaspiro[3.5]nonane (Compound 60)

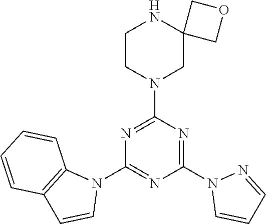 [SMILES: c1ccc2c(c1)ccn2-c1nc(N2CCNC3(COC3)C2)nc(-n2cccn2)n1]

To a solution ethyl 1-(4-chloro-6-(1H-pyrazol-1-yl)-1,3,5-triazin-2-yl)-1H-indole (0.10 g, 0.34 mmol) in anhydrous THF (3 mL) was DIPEA (0.17 g, 1.35 mmol) and 2-oxa-5,8-diazaspiro[3.5]nonae dihydrochloride (0.07 g, 0.37 mmol) under argon atmosphere. The reaction was stirred at 50° C. for 24 hours. The reaction was then extracted with 8:2 dichloromethane/isopropanol mixture (3×20 mL) and washed with sat. NaHCO₃ followed by brine. The combined organic layers were dried over anhydrous Na₂SO₄ and concentrated in vacuo. The residue was purified by Buchi Pureflash chromatography over pre-neutralized silica gel cartridge (40 g) with 100% ethyl acetate to give 8-(4-(1H-indol-1-yl)-6-(1H-pyrazol-1-yl)-1,3,5-triazin-2-yl)-2-oxa-5,8-diazaspiro[3.5]nonane (0.13 g, 98%) as a white solid. ¹H-NMR (400 MHz, DMSO-d₆): δ 8.87 (dd, 1H, J=18, 2.4 Hz), 8.82 (dd, 1H, J=29, 8.4 Hz), 8.39 (dd, 1H, J=10.4, 3.6 Hz), 7.97 (dd, 1H, J=6.8, 1.0 Hz), 7.66 (t, 1H, J=7.2.0 Hz), 7.39 (t, 1H, J=7.2 Hz), 7.26 (m, 1H), 6.84 (dd, 1H, J=13.2, 4.0 Hz), 6.67 (ddd, 1H, J=10.4, 2.4, 1.6 Hz), 4.42 (t, 1H, J=6.0 Hz), 4.38 (m, 3H), 4.16 (d, 2H, J=11.6 Hz), 3.89 (m, 2H), 3.30 (s, 1H), 2.78 (m, 2H). MS (ESI): Calcd. for C₂₀H₂₀N₈O: 388, found 389 (M)⁺

### Example 20: Cellular Assay for Anti-Inflammatory Properties of JMJD3 Inhibitors

The anti-inflammatory properties of five JMJD3 inhibitors (99, 92, 93, 98, and 95, in no particularly order) were evaluated in an immune cellular assay targeting key pro-inflammatory cytokines involved in the pathogenesis of multiple autoimmune disease. The JMJD3 inhibitors that were investigated are displayed in Table 1.

TABLE 1
JMJD3 Inhibitors.

| Compound ID | Structure |
|---|---|
| 99 | 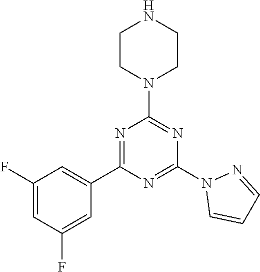 [SMILES: Fc1cc(F)cc(-c2nc(N3CCNCC3)nc(-n3cccn3)n2)c1] |
| 92 | 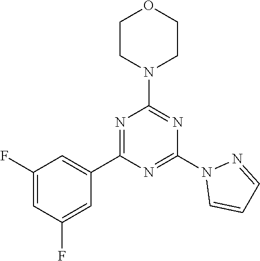 [SMILES: Fc1cc(F)cc(-c2nc(N3CCOCC3)nc(-n3cccn3)n2)c1] |
| 93 | 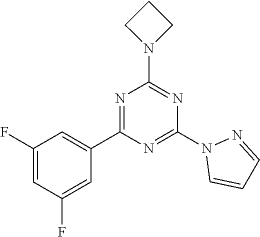 [SMILES: Fc1cc(F)cc(-c2nc(N3CCC3)nc(-n3cccn3)n2)c1] |
| 98 | 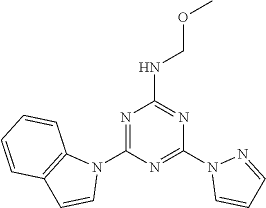 [SMILES: COCNc1nc(-n2cccn2)nc(-n2ccc3ccccc32)n1] |
| 95 | 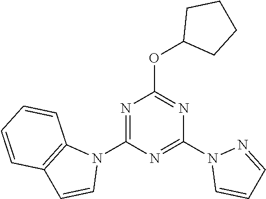 [SMILES: c1ccc2c(c1)ccn2-c1nc(OC2CCCC2)nc(-n2cccn2)n1] |

Specifically, THP-1 human monocytes were grown in 6-well plates (RPMI 1640+10% FBS+1% P/S). After washing with PBS the THP-1 cells were differentiated into macrophages using 20 nM of phorbol 12-myristate 13-acetate (PMA) for 24 hrs. 99, 92, 93, 98, and 95) (1 μM) were added into the macrophages, which are incubated for 4 hours before the addition of 100 ng/mL lipopolysaccharide (LPS) for either 4 or 24 hours. Next, the macrophages are collected via centrifugation at 1.2K rpm for 5 min and use their media to evaluate cytokine protein levels by using the Bio-rad Cytokine ELISA multiplex assay (cat. No M5000031YV). The supernatants from macrophages treated only with only LPS+PMA were collected and used as negative controls. All JMJD3 inhibitors (99, 92, 93, 98, and 95) showed suppression of TNF-alpha (TNFA), interleukin 6 (IL6), interleukin 1beta (IL1B), interleukin 8 (IL8), interleukin 5 (IL5) and interleukin 13 (IL13).

The effects of the JMJD3 inhibitors (99, 92, 93, 98, and 95)) against TNFA, IL6 and IL1B, suggest their use in treatment of autoimmune diseases, including Ulcerative colitis, Crohn's Disease, Rheumatoid Arthritis, Systemic Lupus Erythematosus, Atopic Dermatitis, Psoriasis, Type 1 Diabetes, Graft-versus-host-disease (GVHD), Autoimmune Encephalitis, Bechet's Disease, Sjogren's Syndrome, asthma, lung allergies, and atopic dermatitis. Furthermore, the effects of the JMJD3 inhibitors against IL5 and IL13 suggest their use in asthma, lung allergies and also atopic dermatitis.

### Example 21: Cellular Assay for FoxP3 Induction Activity of JMJD3 Inhibitors

CD4+CD25+FOXP3+ regulatory T cells (Tregs) suppress pro-inflammatory cells and signals and are critical mediators of tolerance, inhibiting excessive inflammation and autoimmunity. Also, treatment of CD4+ T cells with interleukin 2 (IL-2) and transforming growth factor beta (TGF-β) signaling are important for FOXP3 induction and Treg formation.

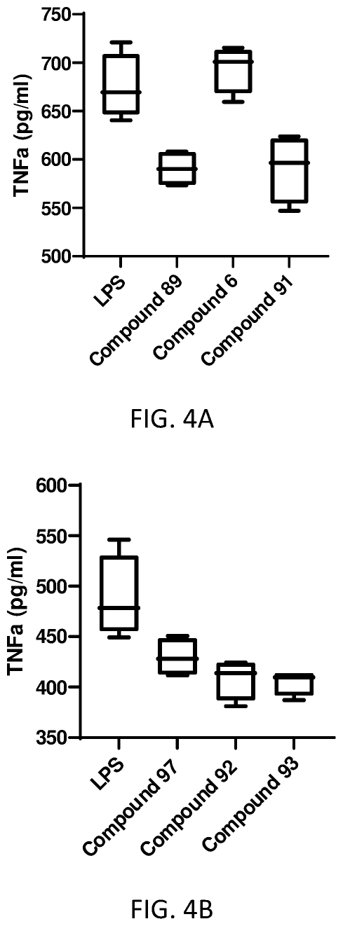

Naïve CD4+ T human cells (cat. no 2W-200, Lonza) were cultured and activated with artificial antigen-presenting cells and soluble anti-CD3 (100 ng/ml) in the presence of IL-2 (300 U/ml) and TGF-β (10 ng/ml) for 5 days. On day 3, cells were transfected with an anti-sense RNA against JMJD3 (cat. No 1299001, Thermo) using Lipofectamine 2000 (cat. No 11668027, Thermo). On day 5, RNA was extracted and qPCR analysis was performed to evaluate FoxP3 mRNA levels by using the following primers: F: 5′-TCC ACA ACA TGC GAC CCC CTT TCA-3′ and R: 5′-ACA GCC CCC TTC TCG CTC TCC A-3′ having a 217 bp product. All JMJD3 inhibitors (99, 92, 93, 98, and 95) in CD4+ T cells induced 7-fold FoxP3 expression levels, thus moving towards differentiation and formation of Tregs. The results of this study are displayed in FIG. 3 (FoxP3 induction). Thus, genetic or pharmacological inhibition of CD4+ T cells will result into FoxP3 activation and induction of Treg formation. Based on this evidence, JMJD3 inhibitors (99, 92, 93, 98, and 95)) could be used in autoimmune diseases where T-regulatory cells have therapeutic potential. There is increasing evidence and literature derived from human clinical studies (Arellano B et al., Regulatory T Cell-based Therapies for Autoimmunity, Discov Med, 22:73-80, 2016).

Thus, the JMJD3 inhibitors 99, 92, 93, 98, and 95 compounds are applicable as therapeutics in graft-versus-host-disease (GVHD), Type 1 Diabetes (T1D), Ulcerative Colitis, Crohn's Disease, Systemic Lupus Erythematosus, Asthma, Rheumatoid Arthritis, multiple sclerosis, encephalomyelitis, Sjogren syndrome, Hashimoto's Thyroiditis, Autoimmune Hepatitis, Behcet's Disease, Castleman disease, Allergic Rhinitis, Eczema, Dressler's Syndrome, Eosinophilic esophagitis, Guillain-Barre Syndrome, Juvenile arthritis, Kawasaki disease, asthma, lung allergies, and atopic dermatitis.

### Example 22: Example of Cellular Assay for Anti-Inflammatory Properties of JMJD3 Inhibitors

THP-1 cells were grown to confluence and plated in 6-well plates. PMA (25 nM) was added to the medium and replaced with non-PMA containing medium 24 hrs later. ATH compounds (1 μM) or vehicle (0.1% DMSO) were added and allowed to incubate. After 4 hrs LPS (50 ng/ml) was added without replacement of the medium and supernatants were collected after 4 and 4 hrs. Media were placed on ice for cytokine detection using ELISA (Quantikine, R&D Systems).

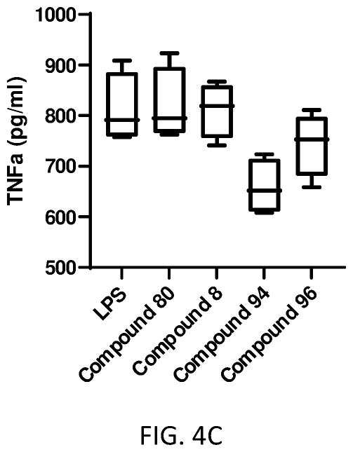

JMJD3 inhibitors Compound 89, Compound 91 (FIG. 4A, p<005), Compound 97, Compound 92, Compound 93 (FIG. 4B, p<0.01), and Compound 94 (FIG. 4C, p<0.01) show suppression of TNF-alpha (TNFA) expression after LPS (lipopolysaccharide) treatment, in THP-1 human monocytes (FIG. 4A-C, p<0.05 for both).

### Example 23: Example JMJD3 Binding Assays

Test compounds were prepared as 111X stocks in 100% DMSO. K_{d} values are determined using an 11-point 3-fold compound dilution series with three DMSO control points. All compounds for K_{d} measurements were distributed by acoustic transfer (non-contact dispensing) in 100% DMSO. The compounds were then diluted directly into the assays such that the final concentration of DMSO was 0.9%. All reactions were performed in polypropylene 384-well plate. Each was a final volume of 0.02 ml. The assay plates were incubated at room temperature with shaking for 1 hour and the affinity beads were washed with wash buffer (lx PBS, 0.05% Tween 20). The beads were then re-suspended in elution buffer (lx PBS, 0.05% Tween 20, 0.5 μM non-biotinylated affinity ligand) and incubated at room temperature with shaking for 30 minutes. The kinase concentration in the eluates was measured by qPCR. Binding constants are calculated with a standard dose-response curve using the Hill equation:

$$Response = Background + \frac{Signal - Background}{1 + ( \frac{{Kd}^{Hill Slope}}{{Dose}^{Hill Slope}} )}$$

The Hill Slope was set to −1. Curves were fitted using a non-linear least square fit with the Levenberg-Marquardt algorithm. The binding data is summarized in Table 2 where A is less than 100 nM, B is 100 to 500 nM, and C is greater than 500 nM.

TABLE 2
JMJD3 Binding constants

| Compound | JMJD3 Affinity (nM) |
|---|---|
| Compound 1 | A |
| Compound 2 | B |
| Compound 89 | A |
| Compound 90 | B |
| Compound 6 | A |
| Compound 91 | B |
| Compound 97 | B |
| Compound 92 | B |
| Compound 93 | A |
| Compound 80 | B |
| Compound 8 | A |
| Compound 94 | B |
| Compound 96 | B |
| Compound 95 | A |
| Compound 60 | A |

### Example 24: Prophetic Example of In Vivo Anti-Inflammatory Efficacy of VNN1 Inhibitors

JMJD3 inhibition ameliorates colitis in studies using the dextran sulfate sodium (DSS)-induced colitis mouse model and downregulation of JMJD3 leds to reduced TNFα expression in mouse macrophages. Our data demonstrate that addition of JMJD3 inhibitors as disclosed herein to THP-1 human monocytes significantly inhibit TNFα release in response to the potent inflammatory inducer LPS.

C57BL/6 mice (20-22 grams) are fed ad lib and assigned to 4 different groups (n=8/group). After a 72 hr acclimation period test groups receive 25 mg/kg of test compounds PO (gavage) in vehicle daily. A control group receives vehicle alone. After 24 hrs, mice are weighed and DSS is added to their drinking water. Mice are then treated with the test compounds and weighed daily and the DSS water is replenished every 72 hrs for 5 days, at which point, it is replaced by regular drinking water. Mice are sacrificed on day twelve and clinical scored was assessed. Intestinal tissue for and blood are also collected. Tissues are either flash frozen and kept in −80° C. for RNA and protein isolation or placed in 10% formalin solution for future histological analysis. Blood is spun at 5K rpm for 5 min and serum is collected and kept at −20° C. for multiplex cytokine analysis. Combined with the central role of TNFα in IBD pathophysiology, the data above are strongly predictive of a beneficial effect of JMJD3 inhibitors in animal models of colitis and subsequently in the amelioration of inflammatory responses in IBD patients. The efficacy data is summarized in Table 3 below where A is strong inhibition, B is moderate inhibition and C is no inhibition.

TABLE 3

| Compound | JMJD3 Affinity (nM) |
|---|---|
| Compound 1 | A |
| Compound 2 | B |
| Compound 89 | A |
| Compound 90 | B |
| Compound 6 | A |
| Compound 91 | B |
| Compound 97 | A |
| Compound 92 | A |
| Compound 93 | A |
| Compound 80 | B |
| Compound 8 | B |
| Compound 94 | A |
| Compound 96 | B |
| Compound 95 | A |
| Compound 60 | A |
| Compound 34 | A |
| Compound 45 | A |
| Compound 52 | B |
| Compound 53 | A |
| Compound 59 | A |
| Compound 68 | A |
| Compound 72 | A |
| Compound 83 | B |
| Compound 84 | A |
| Compound 11 | B |

### Example 25: Prophetic Example of Treating IBD Patients

This is a prophetic example. JMJD3 inhibitors (Compounds 1, 2, 89, 90, 6, 91, 97, 92, 93, 80, 8, 94, 96, and 95) as disclosed in Example 22 are used to treat patients with Ulcerative Colitis and Crohn's Disease patients with active disease, who were sensitive or refractory to previous anti-TNFA treatments. The inhibitor is administered orally as a capsule at a dose of 50-200 mg daily for twelve weeks. After the completion of the treatment. the patients have both clinical and histological improvement, assessed by Mayo or CDAI clinical score, endoscopic appearance, and histological evaluation.

### Example 26: Prophetic Example of Treating Cancer Patients

This is a prophetic example. JMJD3 inhibitors (Compounds 1, 2, 89, 90, 6, 91, 97, 92, 93, 80, 8, 94, 96, and 95) as disclosed in Example 22 are used to treat patients with kidney, breast, castration resistant prostate cancer, acute myeloid leukemia (AML), melanoma. Hodgkin's lymphoma (HL), diffuse large B-cell lymphoma (DLBCL), myelodysplastic syndrome (MDS), esophageal squamous cell carcinoma, head and neck squamous cell carcinoma (HNSCC), and ovarian cancer. The inhibitor is administered orally as a capsule at a dose of 50-200 mg daily in patients with or without chemotherapy. After the completion of the treatment, the patients have clinical improvement, characterized by increased survival after prognosis.

1. A compound, or a pharmaceutically acceptable salt thereof, having a structure represented by Formula (I): 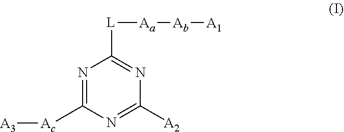
wherein:
L is selected from the group consisting of —C(R¹)₂—, —N—, —NR¹—, —N(R¹)₂, optionally substituted amine(alkyl), —O—, —C(═O)—, —OC(═O)—, —C(═O)O—, optionally substituted heterocyclyl, and a single bond;
Aₐ is selected from the group consisting of optionally substituted C₁-C₆ alkylene and C₁-C₆ alkyl, or Aₐ is not present;
A_{b} is selected from the group consisting of —C(═O)— or —N(CH₃)—, or A_{b} is not present;
A₁ is selected from the group consisting of hydroxyl, amino, optionally substituted C₁₋₁₂ alkyl, optionally substituted C₁-C₁₂ alkoxy, optionally substituted C₁-C₁₂ alkamino, optionally substituted carbamide, C-amido, optionally substituted C₆₋₁₀ aryl, optionally substituted 5-12 membered heteroaryl, optionally substituted C₃₋₁₀ carbocyclyl, and 3-12 membered heterocyclyl, or A₁ is not present;
A₂ is selected from the group consisting of pyrazole and pyridine;
A_{c} is not present;
A₃ is selected from the group consisting of optionally substituted C₁₋₁₂ alkyl, optionally substituted C₆₋₁₀ aryl, optionally substituted 5-12 membered heteroaryl, and optionally substituted C₃₋₁₀ carbocyclyl; and
each instance of R¹, where present, is independently selected from the group consisting of —H, halogen, hydroxy, C₁₋₆ alkyl, C₁-C₆ alkoxy, C₁₋₆ haloalkyl, and C₆₋₁₀ aryl, or, where two R¹ groups are present, the R¹ groups taken together form an optionally substituted 3-12 membered heterocycle or an optionally substituted C₃₋₁₀ carbocyclyl.

2. The compound of claim 1, wherein L is —N(R¹)₂ and the R¹ groups taken together form a 3-12 membered heterocyclyl group.

3. The compound of claim 2, wherein L is a 6-membered heterocyclyl group comprising 1 or 2 heteroatoms.

4. (canceled)

5. (canceled)

6. (canceled)

7. (canceled)

8. The compound of claim 1, wherein L is —NH—.

9. (canceled)

10. The compound of claim 1, wherein Aₐ is C₁-C₃ alkylene.

11. The compound of claim 9, wherein A_{b} is —C(═O)— or is not present.

12. The compound of claim 9, wherein A₁ is amino, hydroxyl, C₁₋₃ alkyl, carbamide optionally substituted with methyl or ethyl, 3 to 6 membered heterocyclyl, 3 to 6 membered heteroaryl, or C₁-C₃ alkoxy.

13. The compound of claim 8, wherein L, Aₐ, A_{b}, and A₁ together provide a structure selected from the group consisting of:

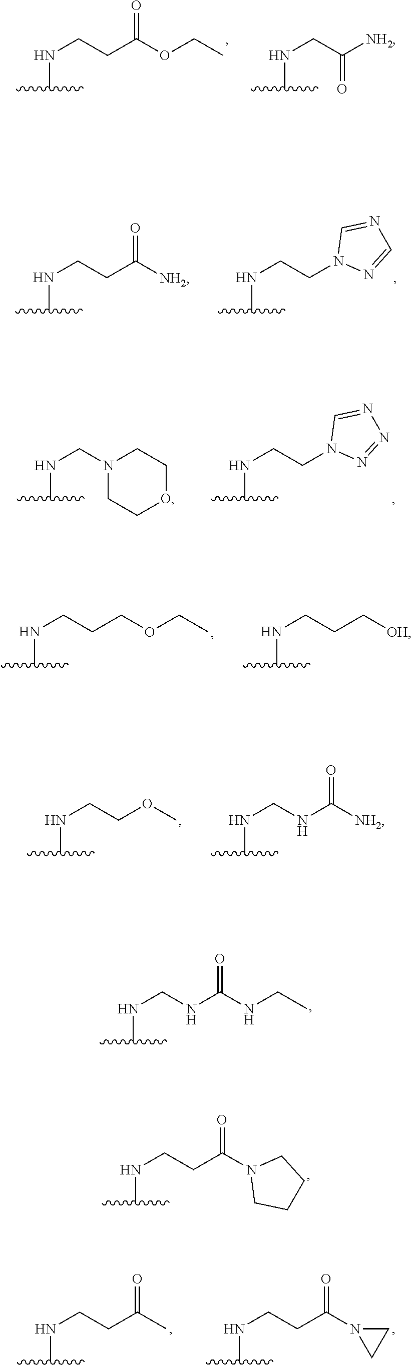

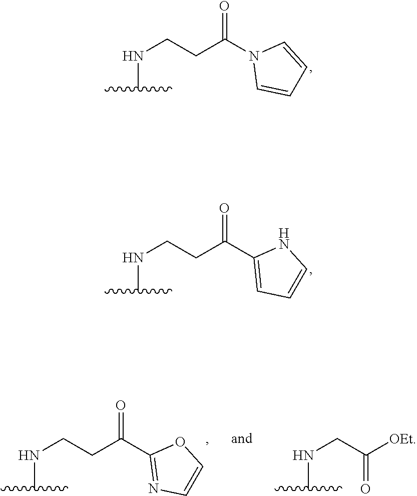 [SMILES: *NCCC(=O)n1cccc1 ; *NCCC(=O)c1ccc[nH]1 ; *NCCC(=O)c1ncco1 ; *NCC(=O)OCC]

14. (canceled)

15. (canceled)

16. (canceled)

17. (canceled)

18. The compound of claim 1, wherein A₂ is pyridine.

19. The compound of claim claim 1, wherein A₂ is

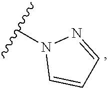 [SMILES: *n1cccn1]

20. (canceled)

21. The compound of claim 1, wherein A₃ is phenyl optionally substituted with —F.

22. The compound of claim 1, wherein A_{c} and A₃ together are

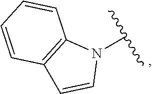 [SMILES: *n1ccc2ccccc21]

23. (canceled)

24. (canceled)

25. (canceled)

26. (canceled)

27. (canceled)

28. (canceled)

29. The compound of claim 1, wherein the compound of Formula (I) is further represented by the structure of Formula (Ia): 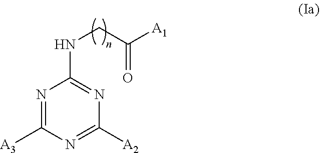
and n is an integer equal to 1, 2, or 3.

30. The compound of claim 29, wherein the structure of Formula (Ia) is represented by a structure selected from the group consisting of Compound 4, 5, 6, 8, 26, 91, and 96.

31. (canceled)

32. (canceled)

33. (canceled)

34. (canceled)

35. The compound of claim 1, wherein the compound of Formula (I) is further represented by the structure of Formula (Id): 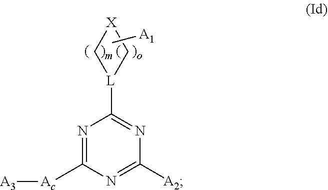
where
X is selected from the group consisting of —CH₂—, —NH—, and —O—;
m is an integer equal to 1, 2, 3, or 4;
o is an integer equal to 1, 2, 3, or 4.

36. The compound of claim 35, further represented by a structure selected from the group consisting of Compound 58, 59, 60, 62, 73, 80, 81, 83, 92, 93, 94, and 99.

37. (canceled)

38. (canceled)

39. (canceled)

40. The compound of claim 1, wherein A₁ is selected from the group consisting of:

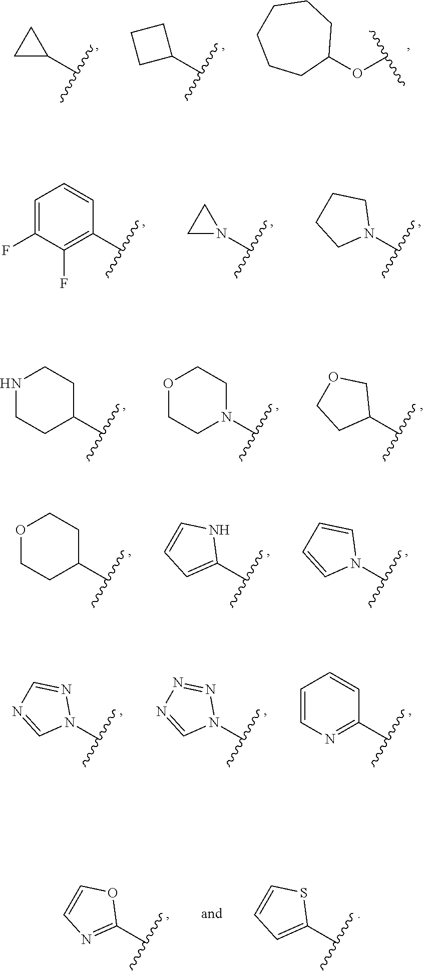

41. (canceled)

42. (canceled)

43. A compound selected from the group consisting of:

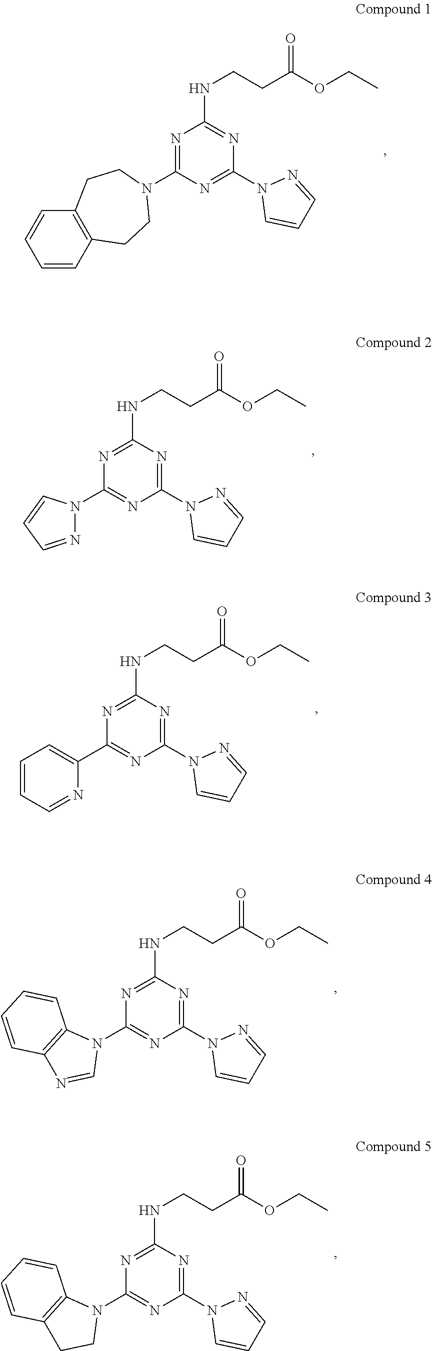 [SMILES: CCOC(=O)CCNc1nc(N2CCc3ccccc3CC2)nc(-n2cccn2)n1 ; CCOC(=O)CCNc1nc(-n2cccn2)nc(-n2cccn2)n1 ; CCOC(=O)CCNc1nc(-c2ccccn2)nc(-n2cccn2)n1 ; CCOC(=O)CCNc1nc(-n2cccn2)nc(-n2cnc3ccccc32)n1 ; CCOC(=O)CCNc1nc(N2CCc3ccccc32)nc(-n2cccn2)n1]

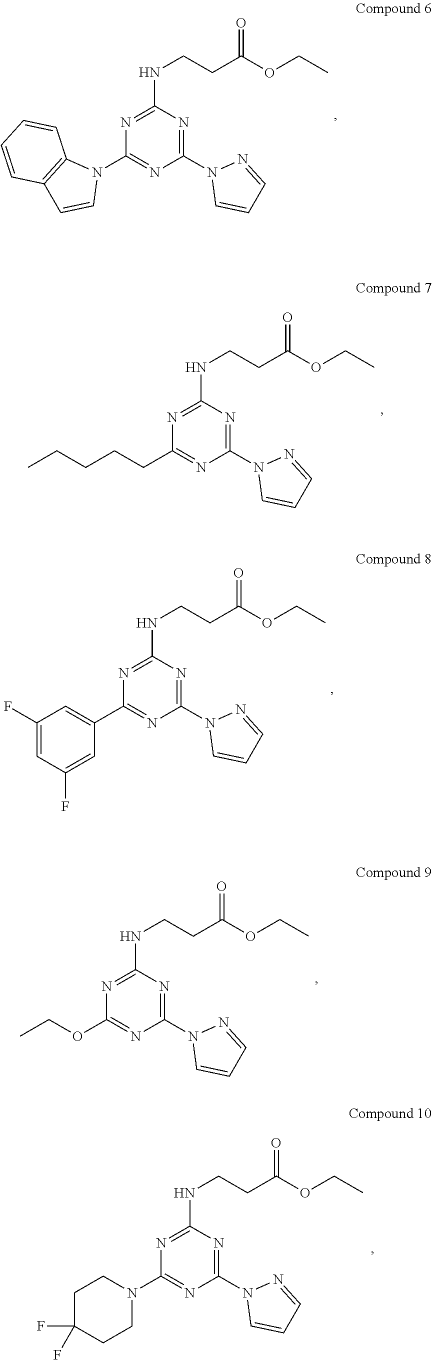

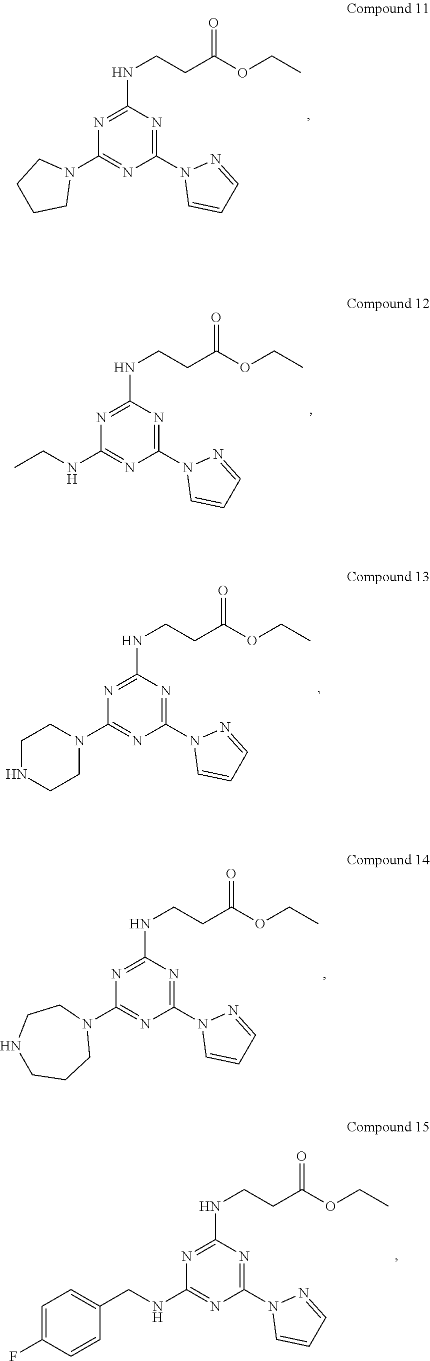

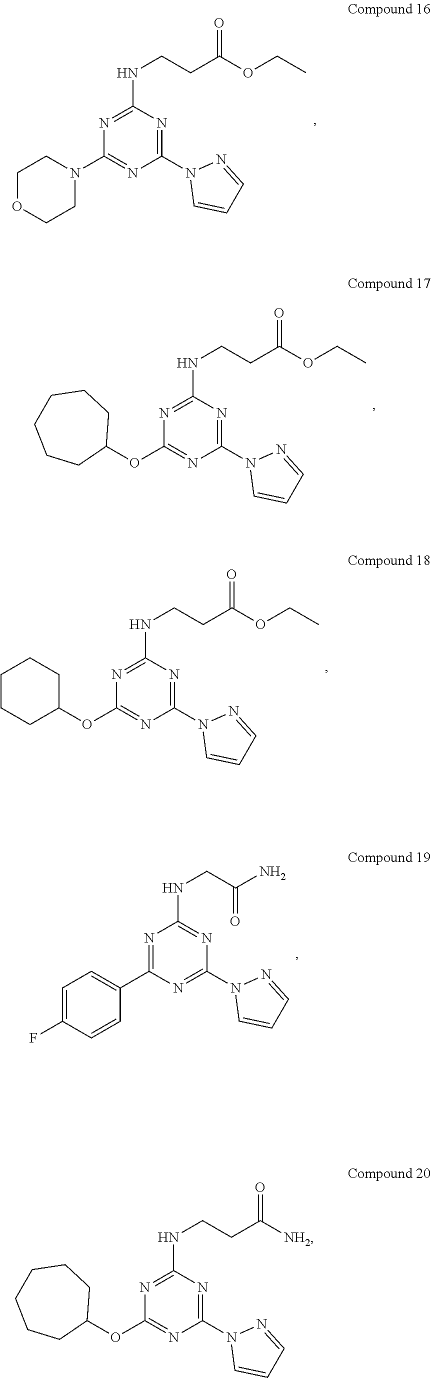 [SMILES: CCOC(=O)CCNc1nc(N2CCOCC2)nc(-n2cccn2)n1 ; CCOC(=O)CCNc1nc(OC2CCCCCC2)nc(-n2cccn2)n1 ; CCOC(=O)CCNc1nc(OC2CCCCC2)nc(-n2cccn2)n1 ; NC(=O)CNc1nc(-c2ccc(F)cc2)nc(-n2cccn2)n1 ; NC(=O)CCNc1nc(OC2CCCCCC2)nc(-n2cccn2)n1]

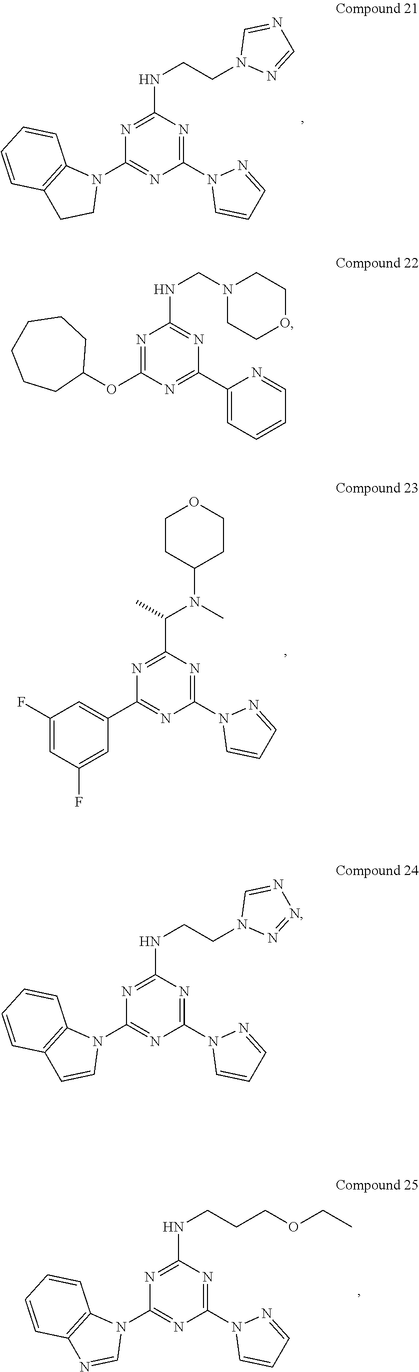 [SMILES: c1ccc2c(c1)CCN2c1nc(NCCn2cncn2)nc(-n2cccn2)n1 ; c1ccc(-c2nc(NCN3CCOCC3)nc(OC3CCCCCC3)n2)nc1 ; C[C@@H](c1nc(-c2cc(F)cc(F)c2)nc(-n2cccn2)n1)N(C)C1CCOCC1 ; c1ccc2c(c1)ccn2-c1nc(NCCn2cnnn2)nc(-n2cccn2)n1 ; CCOCCCNc1nc(-n2cccn2)nc(-n2cnc3ccccc32)n1]

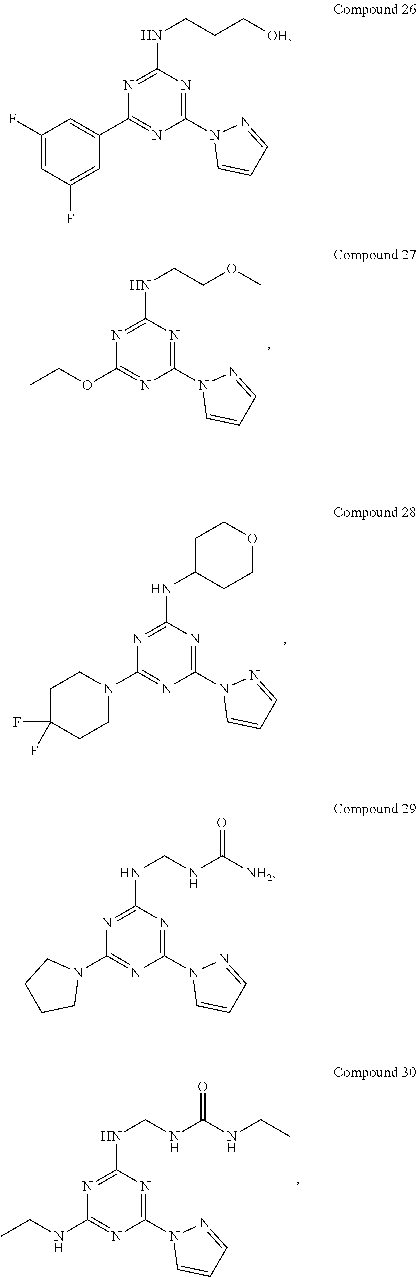

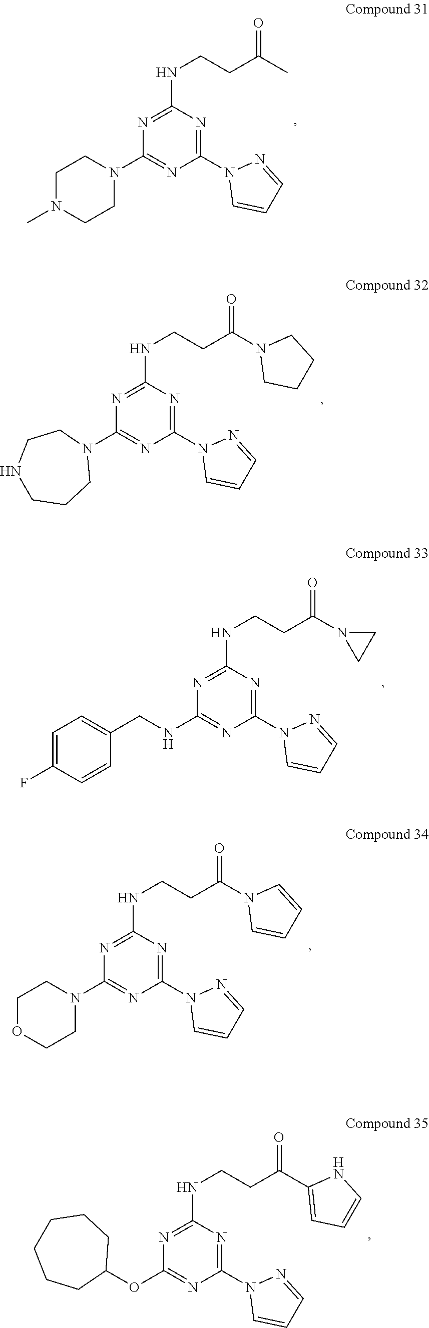 [SMILES: CC(=O)CCNc1nc(N2CCN(C)CC2)nc(-n2cccn2)n1 ; O=C(CCNc1nc(N2CCCNCC2)nc(-n2cccn2)n1)N1CCCC1 ; O=C(CCNc1nc(NCc2ccc(F)cc2)nc(-n2cccn2)n1)N1CC1 ; O=C(CCNc1nc(N2CCOCC2)nc(-n2cccn2)n1)n1cccc1 ; O=C(CCNc1nc(OC2CCCCCC2)nc(-n2cccn2)n1)c1ccc[nH]1]

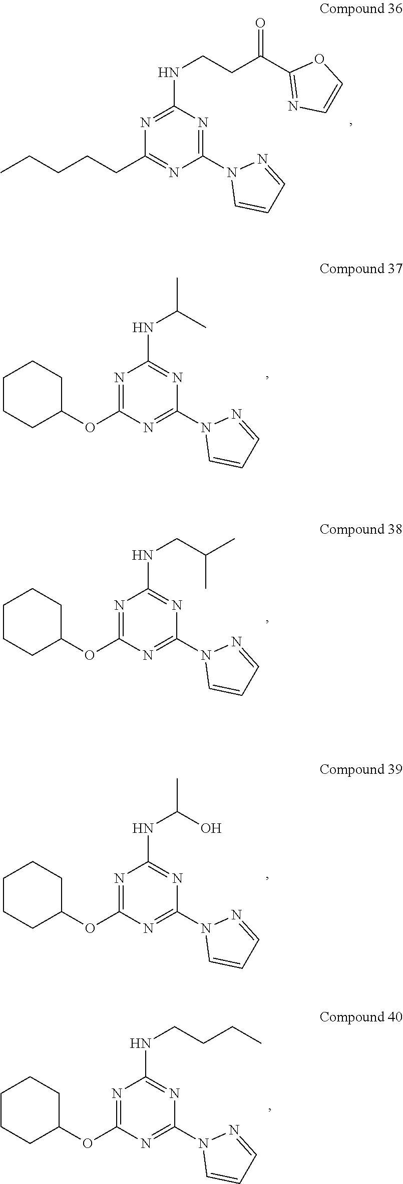 [SMILES: CCCCCc1nc(NCCC(=O)c2ncco2)nc(-n2cccn2)n1 ; CC(C)Nc1nc(OC2CCCCC2)nc(-n2cccn2)n1 ; CC(C)CNc1nc(OC2CCCCC2)nc(-n2cccn2)n1 ; CC(O)Nc1nc(OC2CCCCC2)nc(-n2cccn2)n1 ; CCCCNc1nc(OC2CCCCC2)nc(-n2cccn2)n1]

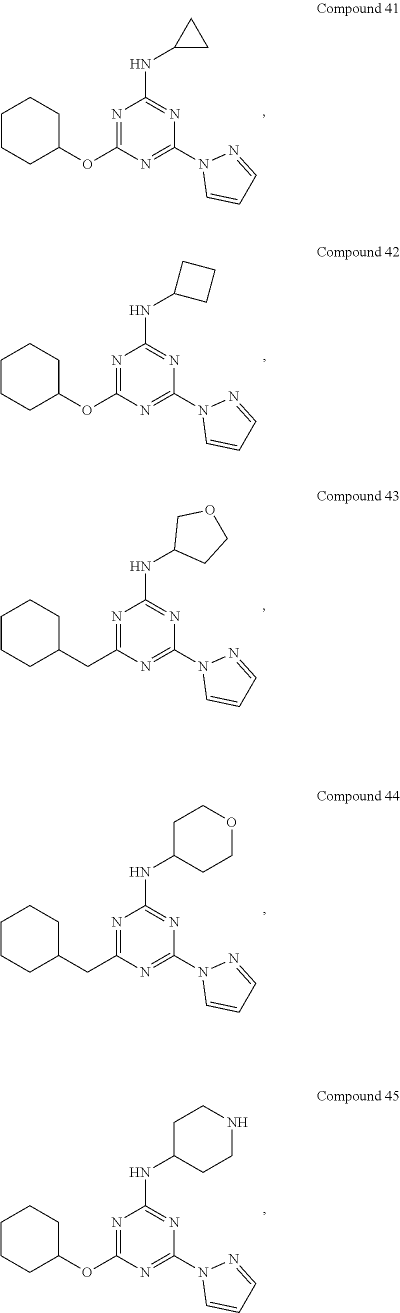 [SMILES: c1cnn(-c2nc(NC3CC3)nc(OC3CCCCC3)n2)c1 ; c1cnn(-c2nc(NC3CCC3)nc(OC3CCCCC3)n2)c1 ; c1cnn(-c2nc(CC3CCCCC3)nc(NC3CCOC3)n2)c1 ; c1cnn(-c2nc(CC3CCCCC3)nc(NC3CCOCC3)n2)c1 ; c1cnn(-c2nc(NC3CCNCC3)nc(OC3CCCCC3)n2)c1]

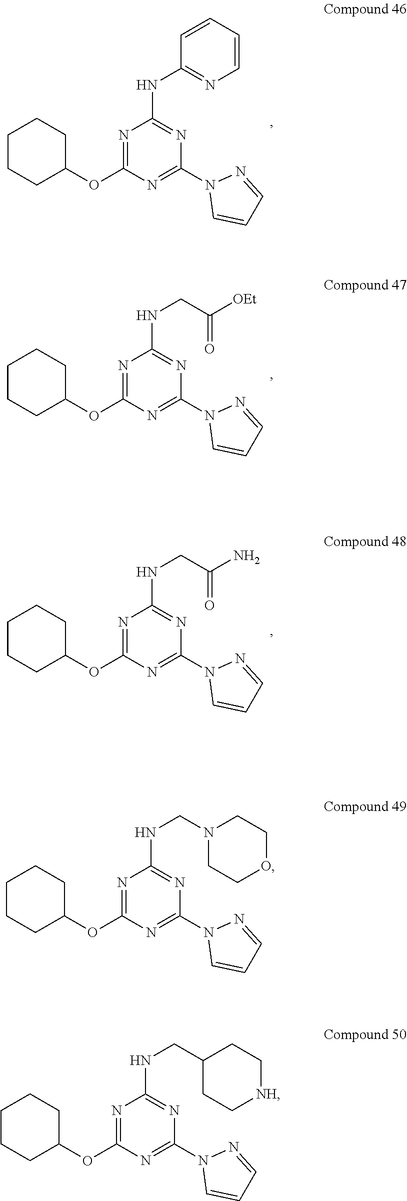 [SMILES: c1ccc(Nc2nc(OC3CCCCC3)nc(-n3cccn3)n2)nc1 ; CCOC(=O)CNc1nc(OC2CCCCC2)nc(-n2cccn2)n1 ; NC(=O)CNc1nc(OC2CCCCC2)nc(-n2cccn2)n1 ; c1cnn(-c2nc(NCN3CCOCC3)nc(OC3CCCCC3)n2)c1 ; c1cnn(-c2nc(NCC3CCNCC3)nc(OC3CCCCC3)n2)c1]

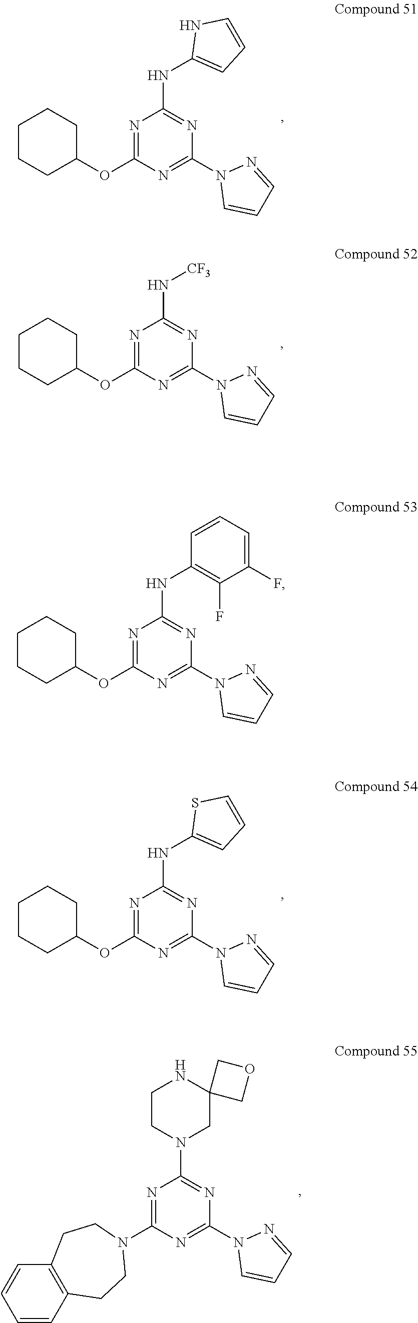 [SMILES: c1c[nH]c(Nc2nc(OC3CCCCC3)nc(-n3cccn3)n2)c1 ; FC(F)(F)Nc1nc(OC2CCCCC2)nc(-n2cccn2)n1 ; Fc1cccc(Nc2nc(OC3CCCCC3)nc(-n3cccn3)n2)c1F ; c1csc(Nc2nc(OC3CCCCC3)nc(-n3cccn3)n2)c1 ; c1ccc2c(c1)CCN(c1nc(N3CCNC4(COC4)C3)nc(-n3cccn3)n1)CC2]

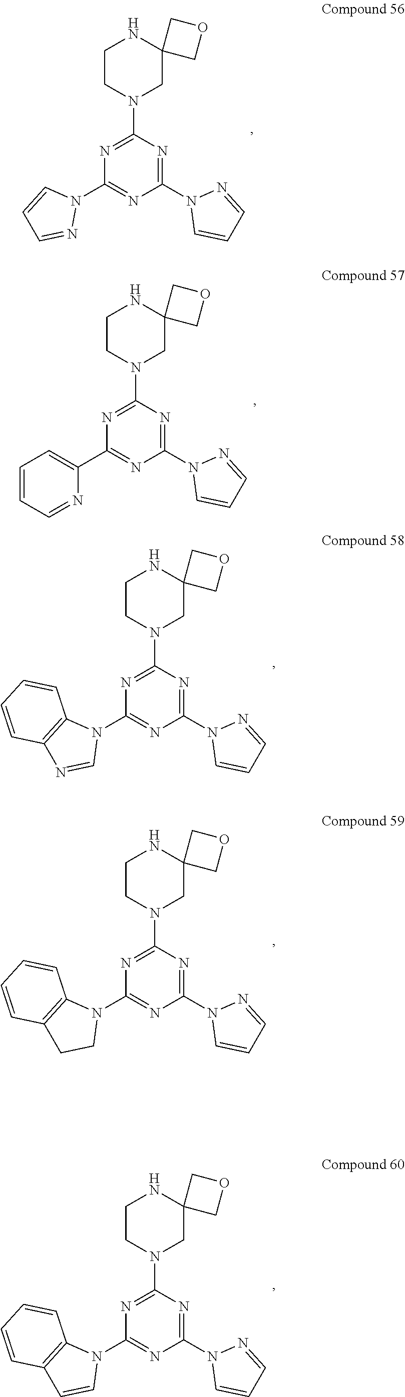 [SMILES: c1cnn(-c2nc(N3CCNC4(COC4)C3)nc(-n3cccn3)n2)c1 ; c1ccc(-c2nc(N3CCNC4(COC4)C3)nc(-n3cccn3)n2)nc1 ; c1ccc2c(c1)ncn2-c1nc(N2CCNC3(COC3)C2)nc(-n2cccn2)n1 ; c1ccc2c(c1)CCN2c1nc(N2CCNC3(COC3)C2)nc(-n2cccn2)n1 ; c1ccc2c(c1)ccn2-c1nc(N2CCNC3(COC3)C2)nc(-n2cccn2)n1]

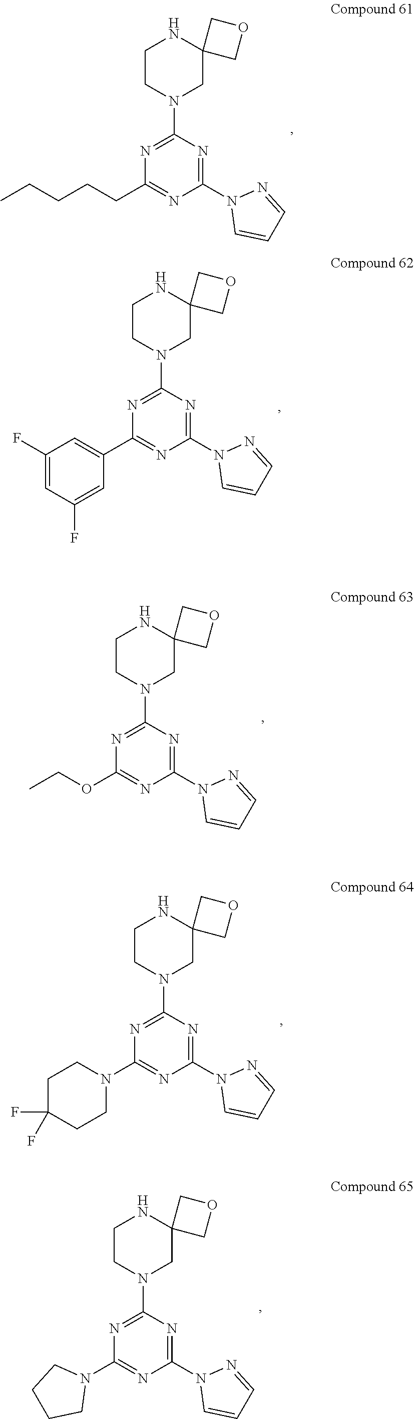 [SMILES: CCCCCc1nc(N2CCNC3(COC3)C2)nc(-n2cccn2)n1 ; Fc1cc(F)cc(-c2nc(N3CCNC4(COC4)C3)nc(-n3cccn3)n2)c1 ; CCOc1nc(N2CCNC3(COC3)C2)nc(-n2cccn2)n1 ; FC1(F)CCN(c2nc(N3CCNC4(COC4)C3)nc(-n3cccn3)n2)CC1 ; c1cnn(-c2nc(N3CCCC3)nc(N3CCNC4(COC4)C3)n2)c1]

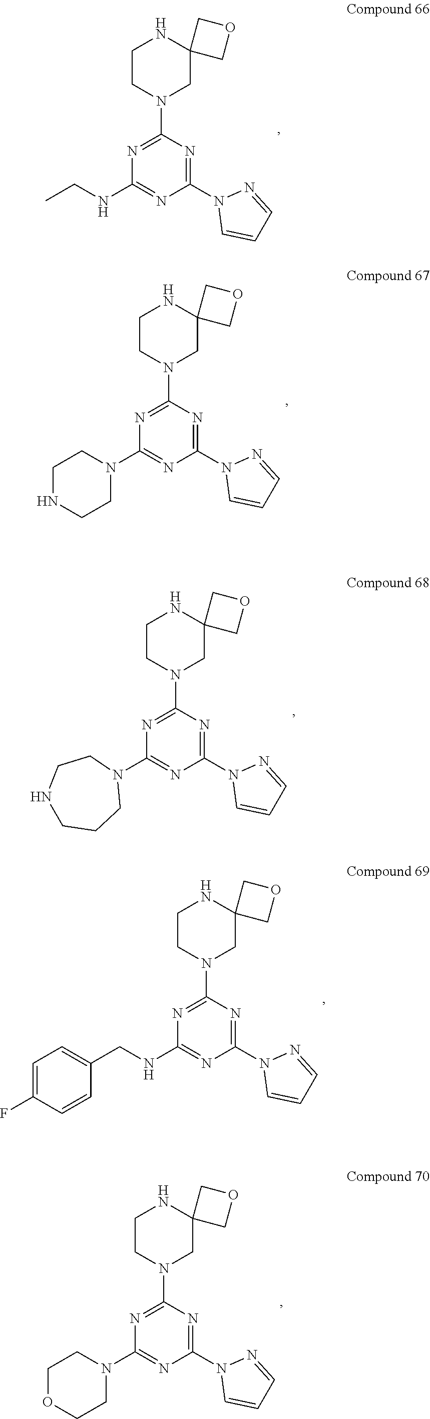 [SMILES: CCNc1nc(N2CCNC3(COC3)C2)nc(-n2cccn2)n1 ; c1cnn(-c2nc(N3CCNCC3)nc(N3CCNC4(COC4)C3)n2)c1 ; c1cnn(-c2nc(N3CCCNCC3)nc(N3CCNC4(COC4)C3)n2)c1 ; Fc1ccc(CNc2nc(N3CCNC4(COC4)C3)nc(-n3cccn3)n2)cc1 ; c1cnn(-c2nc(N3CCOCC3)nc(N3CCNC4(COC4)C3)n2)c1]

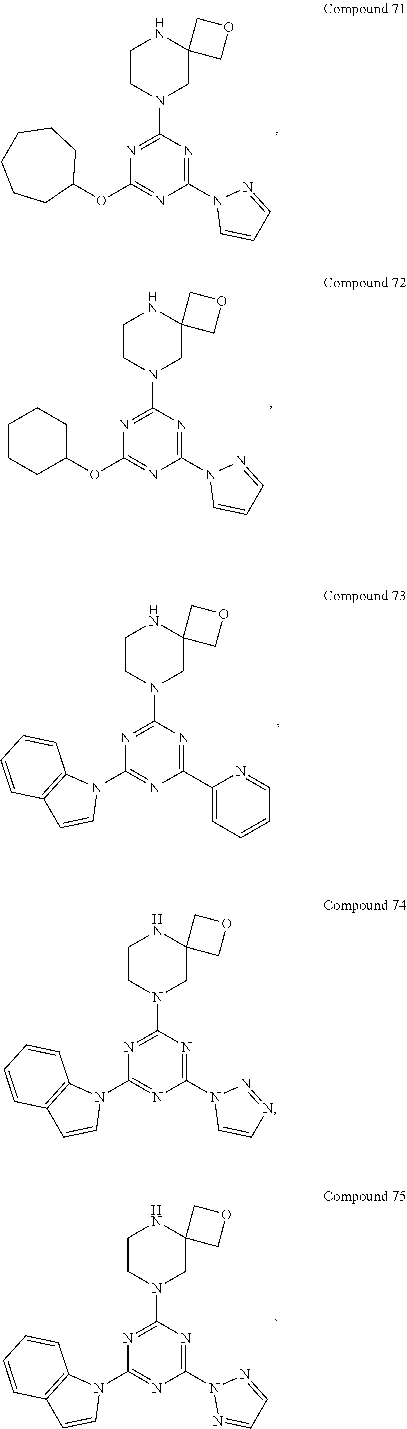 [SMILES: c1cnn(-c2nc(OC3CCCCCC3)nc(N3CCNC4(COC4)C3)n2)c1 ; c1cnn(-c2nc(OC3CCCCC3)nc(N3CCNC4(COC4)C3)n2)c1 ; c1ccc(-c2nc(N3CCNC4(COC4)C3)nc(-n3ccc4ccccc43)n2)nc1 ; c1ccc2c(c1)ccn2-c1nc(N2CCNC3(COC3)C2)nc(-n2ccnn2)n1 ; c1ccc2c(c1)ccn2-c1nc(N2CCNC3(COC3)C2)nc(-n2nccn2)n1]

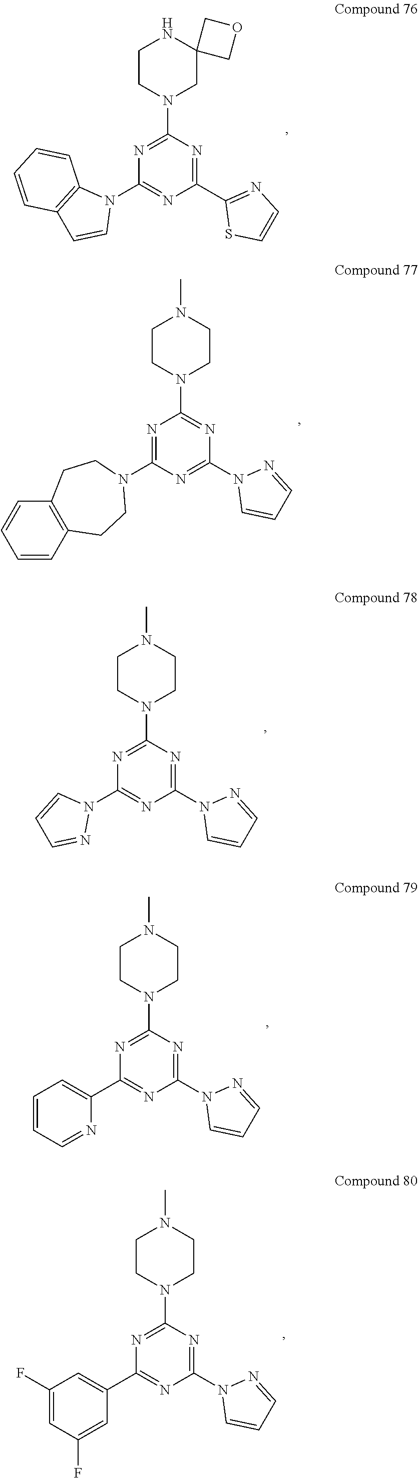 [SMILES: c1ccc2c(c1)ccn2-c1nc(-c2nccs2)nc(N2CCNC3(COC3)C2)n1 ; CN1CCN(c2nc(N3CCc4ccccc4CC3)nc(-n3cccn3)n2)CC1 ; CN1CCN(c2nc(-n3cccn3)nc(-n3cccn3)n2)CC1 ; CN1CCN(c2nc(-c3ccccn3)nc(-n3cccn3)n2)CC1 ; CN1CCN(c2nc(-c3cc(F)cc(F)c3)nc(-n3cccn3)n2)CC1]

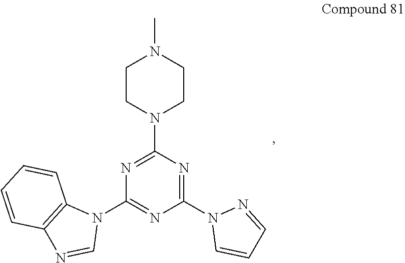 [SMILES: CN1CCN(c2nc(-n3cccn3)nc(-n3cnc4ccccc43)n2)CC1]

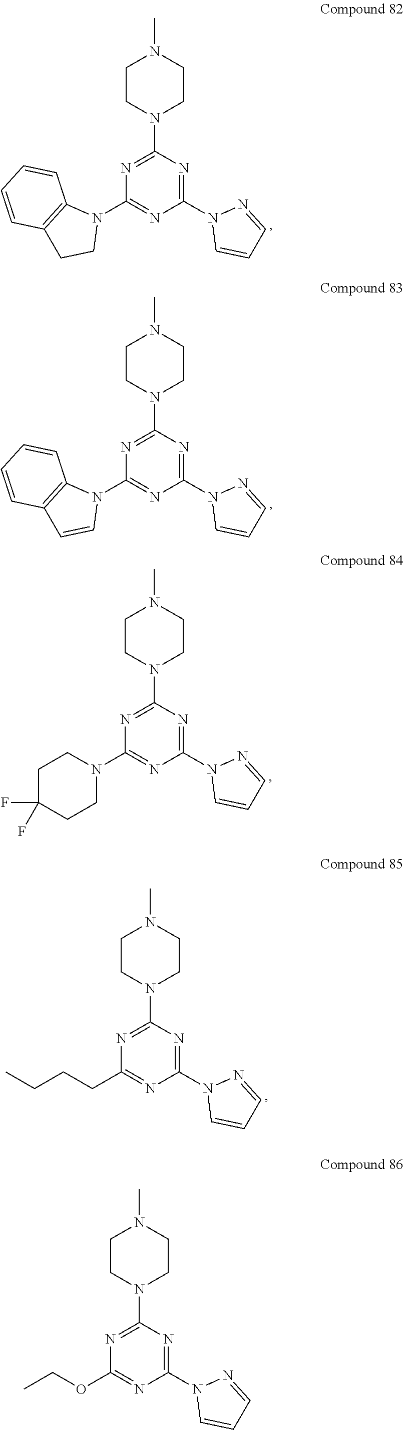 [SMILES: CN1CCN(c2nc(N3CCc4ccccc43)nc(-n3cccn3)n2)CC1 ; CN1CCN(c2nc(-n3cccn3)nc(-n3ccc4ccccc43)n2)CC1 ; CN1CCN(c2nc(N3CCC(F)(F)CC3)nc(-n3cccn3)n2)CC1 ; CCCCc1nc(N2CCN(C)CC2)nc(-n2cccn2)n1 ; CCOc1nc(N2CCN(C)CC2)nc(-n2cccn2)n1]

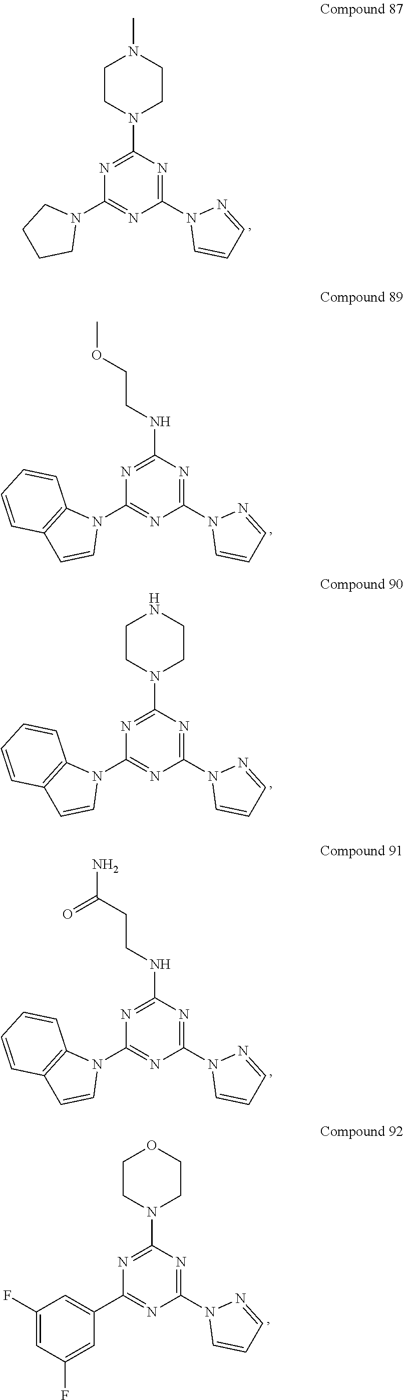 [SMILES: CN1CCN(c2nc(N3CCCC3)nc(-n3cccn3)n2)CC1 ; COCCNc1nc(-n2cccn2)nc(-n2ccc3ccccc32)n1 ; c1ccc2c(c1)ccn2-c1nc(N2CCNCC2)nc(-n2cccn2)n1 ; NC(=O)CCNc1nc(-n2cccn2)nc(-n2ccc3ccccc32)n1 ; Fc1cc(F)cc(-c2nc(N3CCOCC3)nc(-n3cccn3)n2)c1]

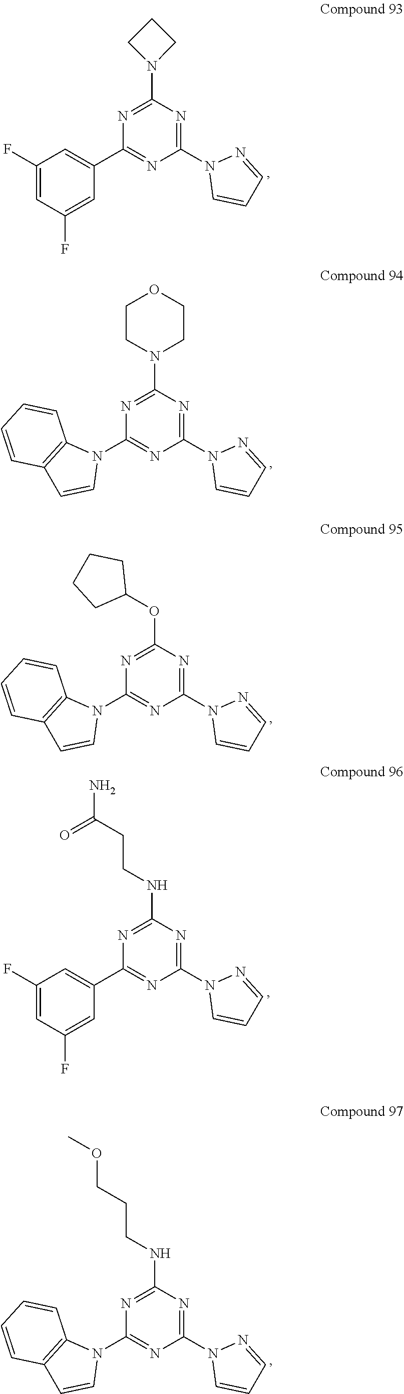

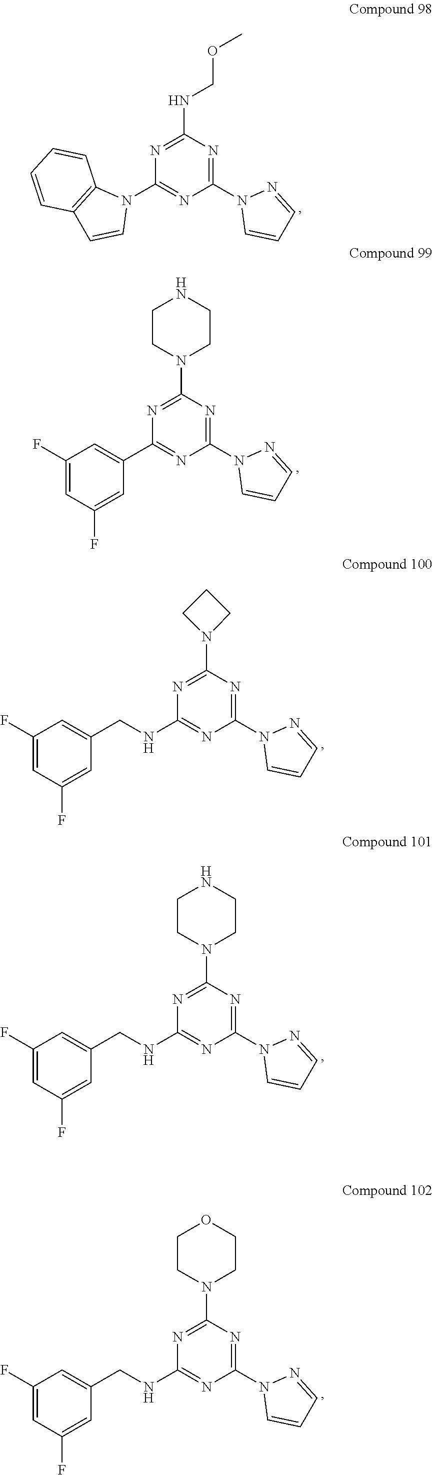 [SMILES: COCNc1nc(-n2cccn2)nc(-n2ccc3ccccc32)n1 ; Fc1cc(F)cc(-c2nc(N3CCNCC3)nc(-n3cccn3)n2)c1 ; Fc1cc(F)cc(CNc2nc(N3CCC3)nc(-n3cccn3)n2)c1 ; Fc1cc(F)cc(CNc2nc(N3CCNCC3)nc(-n3cccn3)n2)c1 ; Fc1cc(F)cc(CNc2nc(N3CCOCC3)nc(-n3cccn3)n2)c1]

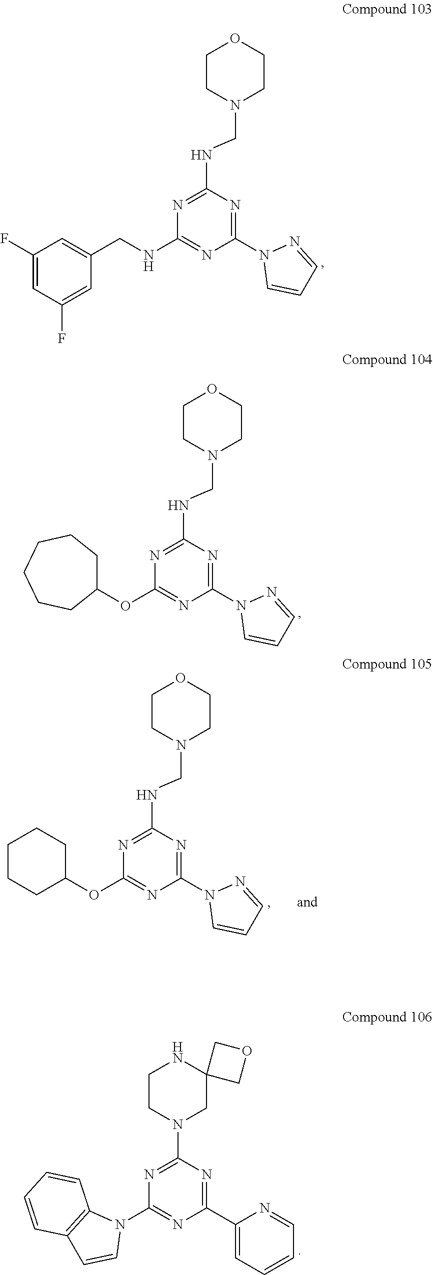 [SMILES: Fc1cc(F)cc(CNc2nc(NCN3CCOCC3)nc(-n3cccn3)n2)c1 ; c1cnn(-c2nc(NCN3CCOCC3)nc(OC3CCCCCC3)n2)c1 ; c1cnn(-c2nc(NCN3CCOCC3)nc(OC3CCCCC3)n2)c1 ; c1ccc(-c2nc(N3CCNC4(COC4)C3)nc(-n3ccc4ccccc43)n2)nc1]

44. A method of treating a JMJD3 mediated condition comprising administering to a subject requiring treatment a compound as described in claim 1.

45. A method of manufacturing a compound as described in claim 1, the method comprising:
displacing a halogen group from cyanuric chloride using H-LAₐA_{b}A₁ in the presence of a base.
